(19) United States
(12) Patent Application Publication
(10) Pub. No.: US 2021/0220489 A1
(43) Pub. Date: Jul. 22, 2021

(54) AMYGDALAR NEURAL ENSEMBLE THAT ENCODES THE UNPLEASANTNESS OF PAIN

(71) Applicant: The Board of Trustees of the Leland Stanford Junior University, Stanford, CA (US)

(72) Inventors: Gregory Scherrer, Chapel Hill, NC (US); Mark Schnitzer, Redwood City, CA (US); Benjamin Grewe, Zurich (CH); Dong Wang, Palo Alto, CA (US); Biafra Ahanonu, San Francisco, CA (US); Gregory Corder, Philadelphia, PA (US)

(21) Appl. No.: 17/148,377

(22) Filed: Jan. 13, 2021

Related U.S. Application Data
(60) Provisional application No. 62/962,581, filed on Jan. 17, 2020.

(51) Int. Cl. A61K 49/00; A61K 9/00; C07K 16/28; C12N 15/113

(52) U.S. Cl. CPC A61K 49/0004; A61K 9/0019; C07K 16/28; C12N 2310/141; C07K 2317/76; C12N 2310/11; C12N 2310/14; C12N 15/1138

(57) ABSTRACT

An ensemble of neurons in the basolateral amygdala (BLA) has been identified that encodes nociceptive information across pain modalities, including pain evoked by noxious thermal and mechanical stimuli. Methods are provided for screening candidate agents for inhibition of neural activity of the BLA nociceptive ensemble. Screening assays further include determining the effectiveness of candidate agents in alleviating pain and reducing aversive pain avoidance behavior.

### STATEMENT REGARDING FEDERALLY SPONSORED RESEARCH OR DEVELOPMENT

This invention was made with Government support under contracts DA031777, NS106301, DA043609, DA041029, and DA035165 awarded by the National Institutes of Health. The Government has certain rights in the invention.

### BACKGROUND OF THE INVENTION

Pain is both a sensory and affective experience (Price, Science 288, 1769-1772 (2000)). The unpleasant percept that dominates the affective dimension of pain is coupled with the motivational drive to engage protective behaviors that limit exposure to noxious stimuli (Baliki et al., Neuron 87, 474-491 (2015)). Although previous work has uncovered detailed mechanisms underlying the sensory detection of noxious stimuli and spinal processing of nociceptive information (Peirs et al., Science 354, 578-584 (2016)), how brain circuits transform emotionally inert information ascending from the spinal cord into an affective pain percept remains unclear (Garcia-Larrea et al., Prog. Neuropsychopharmacol. Biol. Psychiatry (2017)). Attaining a better understanding of the mechanisms underlying pain affect is important, because it could lead to novel therapeutic strategies to limit the suffering of patients with chronic pain.

The amygdala critically contributes to the emotional and autonomic responses associated with valence coding of neural information, such as responses during fear or pain (Janak et al., Nature 517, 284-292 (2015)). Damage to the basolateral amygdala (BLA) can induce a rare phenomenon in which noxious stimuli remain detected and discriminated but are devoid of perceived unpleasantness and do not motivate avoidance (Hebben et al., Behav. Neurosci. 99, 1031-1039 (1985); Neimann et al., Bull. Soc. Fr. Dermatol. Syphiligr. 71, 292-294 (1964)). Conversely, impairment of somatosensory cortex function reduces the ability to both localize noxious stimuli and describe their intensity, without altering aversion or avoidance (Ploner et al., Pain 81, 211-214 (1999); Uhelski et al., Pain 153, 885-892 (2012)). Thus, BLA affective neural circuits might link nociceptive inputs to aversive perceptions and behavior selection.

Patients with chronic pain often suffer allodynia, a pathological state in which an intense unpleasant percept arises in response to innocuous stimuli such as light touch (Costigan et al., Annu. Rev. Neurosci. 32, 1-32 (2009)). Notably, the BLA displays heightened activity during chronic pain (Neugebauer, Amygdala Pain Mechanisms. Handb. Exp. Pharmacol. 227, 261-284 (2015)), and longitudinal functional magnetic resonance imaging studies in humans and rodents show that neural hyperactivity and altered functional connectivity in the amygdala parallel the onset of chronic pain, suggesting that the BLA might play a critical role in shaping pathological pain perceptions (Chang et al., Pain 158, 488-497 (2017); Simons et al., Pain 155, 1727-1742 (2014); Hashmi et al., Brain 136, 2751-2768 (2013)). However, it remains unclear how the BLA influences the unpleasant aspects of innate acute and chronic pain perceptions (Gore et al., Cell 162, 134-145 (2015)), while the role of nociceptive circuits in the central amygdala are better understood (Neugebauer, et al., J. Neurosci. 23, 52-63 (2003); Han, et al., Cell 162, 363-374 (2015)).

### SUMMARY OF THE INVENTION

The inventors identified an ensemble of neurons in the basolateral amygdala (BLA) that encodes nociceptive information. BLA neurons responsive to nociceptive stimuli were identified by tracking the somatic Ca²⁺ dynamics of individual BLA Camk2a⁺ principal neurons in mice presented with diverse noxious and innocuous stimuli. Noxious heat, cold, and pin prick stimuli elicited significant Ca²⁺ responses in the identified BLA neurons. Alignment of all stimulus-evoked ensemble responses to the noxious heat trials revealed an overlapping population of principal neurons that encode nociceptive information across pain modalities (i.e., noxious heat, cold, pin), which are referred to herein as the BLA nociceptive ensemble (see Examples).

In one aspect, a method of treating a subject for pain is provided, the method comprising administering a therapeutically effective amount of an agent that disrupts neural activity of one or more neurons of a BLA nociceptive ensemble in the brain of the subject. In some embodiments, the agent disrupts neural activity of a subset of neurons in the BLA nociceptive ensemble. In some embodiments, the subset comprises or consists of a nociceptive-specific subpopulation of neurons. In other embodiments, the agent disrupts neural activity of all of the neurons of the BLA nociceptive ensemble.

In certain embodiments, the agent is administered in an amount sufficient to attenuate pathological or neuropathic pain.

In certain embodiments, the agent is administered in an amount sufficient to relieve allodynia or hyperalgesia, including, without limitation, thermal, mechanical, or opioid-induced allodynia or hyperalgesia.

In certain embodiments, the agent is administered in an amount sufficient to reduce aversive pain avoidance behavior.

In certain embodiments, the nociceptive ensemble comprises c-Fos⁺ mid-anterior BLA Camk2a⁺ principal neurons that are activated by nociceptive stimuli.

In certain embodiments, the pain is acute pain or chronic pain.

In certain embodiments, the agent is administered locally to the BLA nociceptive ensemble. In some embodiments, the agent is administered locally by stereotactic injection into the BLA nociceptive ensemble in the brain of the subject.

In another aspect, a method of screening for an agent that modulates neural activity in a BLA nociceptive ensemble in a brain of a subject is provided, the method comprising: a) contacting the BLA nociceptive ensemble with a candidate agent; and b) measuring neural activity in the BLA nociceptive ensemble in response to the candidate agent.

In certain embodiments, the method further comprises monitoring pain perception in the subject to determine if the candidate agent modulates pain perception.

In certain embodiments, the neural activity in the BLA nociceptive ensemble and/or pain perception is monitored in response to a test stimulus. For example, the test stimulus may be a noxious stimulus or an innocuous stimulus. In some embodiments, the noxious stimulus is a noxious mechanical (e.g., noxious pin prick or filament) or thermal stimulus (e.g., noxious heat or noxious cold). In some embodiments, the innocuous stimulus is light touch.

In certain embodiments, reduced pain perception in response to the noxious stimulus in the presence of the candidate agent compared to in the absence of the candidate agent indicates that the candidate agent has analgesic activity.

In certain embodiments, the method further comprises monitoring the subject for reduced pain affective-motivational behavior in the presence of the candidate agent compared to in the absence of the candidate agent.

In certain embodiments, the candidate agent is a small molecule, a peptide, a protein, an aptamer, an antibody, an antibody mimetic, a receptor ligand, or an inhibitory nucleic acid that modulates neural activity of at least a subset of neurons in the BLA nociceptive ensemble. In some embodiments, the antibody is selected from the group consisting of a polyclonal antibody, a monoclonal antibody, a chimeric antibody, a humanized antibody, a F(ab) fragment, a F(ab′)2 fragment, a Fᵥ fragment, and a nanobody. In some embodiments, the inhibitory nucleic acid is selected from the group consisting of a small interfering RNA (siRNA), a microRNA (miRNA), a Piwi-interacting RNA (piRNA), a small nuclear RNA (snRNA), an antisense oligonucleotide, and a peptide nucleic acid.

In certain embodiments, pain perception is monitored in the subject using a mechanical withdrawal test, an electronic Von Frey test, a manual Von Frey test, a Randall-Selitto test, a Hargreaves test, a hot plate test, a cold plate test, a thermal probe test, an acetone evaporation test, a cold plantar test, a temperature preference test, a grimace scale test, or weight bearing and gait analysis.

Candidate agents, identified by screening, as described herein, may be useful in alleviating pain and pain affective-motivational behavior.

In another aspect, a method of mapping nociceptive and aversive responses to neurons in a basolateral amygdala (BLA) nociceptive ensemble in the brain of a subject is provided, the method comprising: a) imaging neural activity within the BLA nociceptive ensemble associated with nociceptive and aversive responses to a test stimulus; and b) mapping responsive neurons exhibiting the neural activity. In certain embodiments, the neural activity is Ca²⁺ transient activity of one or more neurons in the BLA nociceptive ensemble.

### BRIEF DESCRIPTION OF THE DRAWINGS

FIGS. 1A-1M show that a distinct nociceptive neural ensemble in the BLA represents diverse painful stimuli. (FIG. 1A) BLA neural activity was imaged in freely behaving mice with a microendoscope and the virally expressed fluorescent Ca²⁺ indicator GCaMP6m. Noxious mechanical (pin prick) and thermal (55° C. H₂O and 5° C. H₂O or acetone) stimuli were delivered to the left hindpaw, while reflexive and affective-motivational behavior were monitored via a scope-mounted accelerometer. (FIG. 1B) Microendoscope placement and GCaMP6m expression in the right, contralateral BLA. The red line marks the focal plane and is also a 1.0-mm scale bar. (FIGS. 1C and 1D) Map of active BLA neurons (n=131 neurons) with numbers in (FIG. 1C) matching independent component analysis-derived neuron activity traces in (FIG. 1D). Scale bar, 100 mm. (FIG. 1E) Spearman's correlation between reflexive withdrawal and affective-motivational escape acceleration. (FIG. 1F) Mean Ca²⁺ response (Z-scored AF/F per trial) across all trials for all BLA neurons imaged during a single session (n=215 neurons) from the same animal. Neurons are aligned from high to low Ca²⁺ responses in the noxious heat trials. Individual neuron identifications between different stimuli are consistent across the trial rows. (FIG. 1G) Stimulus-locked mean Ca²′ activity within the nociceptive ensemble (cyan) and mean affective-motivational escape acceleration (red). Shaded region, ±SEM. Pie charts indicate the percentages of significantly responding neurons. (FIG. 1H) Venn diagram of neural populations encoding nociceptive information in response to noxious heat, cold, and pin stimuli. Numbers show means±SEM of percentages of significantly responding neurons across imaging sessions (see FIG. 9E). (FIG. 1I) Neural populations within the nociceptive ensemble that encode innocuous light touch (0.07-g filament) and mild touch (a 1.4- or 2.0-g filament). (FIG. 1J) Divergent neural populations (versus the nociceptive ensemble) encoding appetitive stimuli (10% sucrose consumption). (FIG. 1K) Overlapping BLA populations between the nociceptive ensemble, electric footshock, and aversive stimuli (isopentylamine odor, facial air puff, 85-dB noise, and quinine consumption). A subset of nociceptive ensemble neurons was pain specific (˜6% of the BLA neurons). (FIG. 1L) Accuracies of a nine-way Naïve Bayes decoder that distinguishes the ensemble activities for noxious, innocuous, aversive, anticipatory, and appetitive stimuli. The percentage of decoder accuracy to output for the actual stimuli (diagonal) was compared to that for the incorrect stimuli (off the diagonal) and normalized so that each actual stimuli column added up to 100%. Stars on the diagonal indicate the correct prediction of said stimulus was significantly greater than all off-diagonal stimuli within the same column (Wilcoxon sign-rank, Benjamini-Hochberg corrected). (FIG. 1M) Spearman's correlation (r) between per trial pain behavioral responses and nociceptive ensemble activation. Error bars, ±SEM per session animal responses; n=9 mice, 3 to 4 sessions each.

FIGS. 2A-2L show the BLA nociceptive ensemble is necessary for generating protective and avoidance behavioral responses to painful stimuli. (FIG. 2A) Experimental strategy for inhibiting BLA nociceptive ensemble activity. Nociception-mediated targeted recombination in activity neural populations (noci-TRAP) of the inhibitory DREADD (hM4) receptor. CNO, clozapine N-oxide; 4-OHT, 4-hydroxytamoxifen. (FIG. 2B) noci-TRAPhM4 expression in the BLA nociceptive ensemble. CeA, central amygdala; ITC, intercalated neurons; Pir, piriform cortex. Scale bar, 50 mm. (FIG. 2C) Quantification of BLA noci-TRAPeYFP neurons following either no stimulus, innocuous touch (0.07-g filament), or noxious pin prick stimulation; n=6 mice/group. (FIGS. 2D and 2E) Effect of inhibiting the BLA nociceptive ensemble against reflexive behaviors, demonstrated by a von Frey mechanical threshold assay (FIG. 2D) and reflexive withdrawal frequency to increasing noxious mechanical stimuli (FIG. 2E). n=14 mice per group. (FIGS. 2F and 2G) Effect of inhibiting the BLA nociceptive ensemble against pain affective-motivational behaviors in response to increasingly noxious mechanical (FIG. 2F) and thermal stimuli (FIG. 2G). n=14 mice per group. (FIG. 2H) Effect of inhibiting the BLA nociceptive ensemble on adaptive avoidance behavior to noxious thermal environments. The kymograph displays mouse location on a thermal gradient track over a 60-min trial following administration of saline (n=6 mice) or CNO (n=7 mice). Noxious temperature zones were areas at <17° C. and >42° C. (FIG. 21) Total number of visit entries (gray and light blue lines) and the occupancy time (black and dark blue lines) in the track's 25 thermal zones. (FIG. 2J) Temporal cumulative visits and the mean occupancy time per visit (inset) to the noxious hot and cold zones. (FIG. 2K) Occupancy time within the open arms of an elevated plus maze (EPM). (FIG. 2L) The 10% sucrose spout lick rates and preference over a water choice. Overlaid dots and lines represent individual animals. Error bars, ±SEM. For (FIG. 2C) and (FIG. 2E) to (FIG. 2G) (CNO group baseline time points only), one-way analysis of variance (ANOVA; Friedman's) plus Dunn's correction. For (FIG. 2D) to (FIG. 2G) and (FIG. 21), two-way repeated measures ANOVA with Bonferroni correction. For (FIG. 2J) and (FIG. 2K), data on left analyzed with Kolmogorov-Smirnov test; data on right analyzed with Student's t test. Star, P<0.05.

FIGS. 3A-3H show convergence of BLA neural ensemble representations of innocuous and noxious information during chronic pain. (FIG. 3A) Long-term tracking of BLA neural activity with microendoscopes throughout the development of chronic neuropathic pain. Peripheral nerve injury results in an increased sensitivity and perceived aversion to innocuous (allodynia) and noxious (hyperalgesia) stimuli. (FIG. 3B) Affective-motivational escape acceleration for neuropathic (top row; n=5 mice) and uninjured (bottom row; n=4 mice) animals in response to noxious pin or light touch stimuli before and after nerve injury. Dark lines, means; shaded regions, ±SEM. (FIG. 3C) Hyperalgesic and allodynic behavioral responses in neuropathic (n=13 mice for paw withdrawal, n=5 mice for escape acceleration) or uninjured (n=4 mice for both measures) animals after application of light touch (0.07-g filament), noxious pin, or noxious cold (acetone or 5° C. H₂O drop) stimuli, respectively. Data were quantified by reflexive hypersensitivity (left axis) and affective-motivational escape acceleration (right axis). (FIG. 3D) Mean Ca²⁺ activity (Z-scored AF/F per trial) of all neurons from the same animal for that imaging session, before and after nerve injury, in response to noxious pin prick, noxious cold, and light touch stimuli. Neuron identifications were consistent between stimuli within a day, but not across days (n=157 and 156 neurons, for days −7 and 42, respectively). (FIG. 3E) Mean Ca²⁺ response within the nociceptive ensemble for neuropathic (top row; n=13 mice, 12,026 total neurons imaged) and uninjured (bottom row; n=4 mice, 5370 total neurons imaged) animals in response to noxious pin or light touch stimuli. (FIG. 3F) Venn diagrams of percentages of significantly responding neurons to noxious pin, noxious cold, and light touch before and after nerve injury. (FIG. 3G) Overlapping neural populations responsive to light touch within the nociceptive ensemble (pin prick and 5° C. water or acetone responsive neurons) after nerve injury (n=13 mice) or in uninjured animals (n=4 mice). Numbers indicate means±SEM. (FIG. 3H) Percentages of nociceptive ensemble activated and escape acceleration per imaging session (light-colored points) and across animal groups and conditions (dark, larger points) show significant correlations [Spearman's r=0.54 (normal), 0.33 (Neuropathic), and 0.58 (Uninjured) groups]. All test results in the figure were analyzed via Wilcoxon rank-sum with Benjamini-Hochberg correction unless otherwise noted. Stars, P<0.01.

FIGS. 4A-4G show inhibition of neuropathic BLA ensemble activity reduces the aversive quality of chronic pain. (FIG. 4A) Utilization of light touch to gain genetic access to, and manipulate, the neuropathic nociceptive ensemble. (FIG. 4B) Quantification of light touch TRAP neurons in the BLA of neuropathic mice compared to uninjured mice; n=7 per group. (FIG. 4C) Behavioral raster plots from neuropathic mice showing the effects of inhibiting the BLA nociceptive ensemble on reflexive and affective-motivational pain behaviors associated with cold allodynia. (FIGS. 4D and 4E) Summary of the effects of ensemble inhibition against reflexive (FIG. 4D) and affective-motivational (FIG. 4E) pain behaviors in response to noxious pin prick, noxious cold (acetone drop), or formerly innocuous touch stimuli (0.07-g filament). Behavior was assessed before and 42 days after nerve injury and again at 60 min after CNO or saline administration on day 42; n=14 per group. (FIGS. 4F and 4G) Effects of neuropathic ensemble inhibition on adaptive avoidance during a cold place aversion assay. (FIG. 4F) Group mean exploration paths, color coded for the relative occupancy time, following CNO or saline treatments; (FIG. 4G) summary of the effects in response to decreasing floor plate temperatures; n=6 per group. Stars, P<0.05 for all panels. In (FIG. 4G), the black star indicates P<0.05 versus the uninjured+saline group; open star, P<0.05 versus the neuropathic+saline group. Overlaid dots and lines represent individual subjects. Error bars, ±SEM. For (FIG. 4B), Student's t test; (FIGS. 4D and 4E), two-way ANOVA with Bonferroni correction; (FIG. 4G) three-way ANOVA with Bonferroni correction.

FIGS. 5A-5E show molecular and anatomical characterization of neurons constituting the BLA nociceptive neural ensemble. (FIG. 5A) Unilateral, left hindpaw pin prick-induced c-Fos mRNA expression in the ipsilateral left and contra-lateral right BLA. (Two-way ANOVA, Bonferroni post-hoc). Stars, at least P<0.05. (FIG. 5B) Anterior to posterior quantification of nociceptive c-Fos-i-neurons in the right BLA (left blue y-axis), and the percentage of nociceptive c-Fos-i-neurons that are Camk2a-i-principal neurons (right red y-axis). (FIG. 5C) Representative triple fluorescence in situ hybridization (FISH) for identifying nociceptive (c-Fos), principal (Camk2a), and GABAergic (Vga) neurons in the BLA. (FIGS. 5D and 5E) Quantification of nociceptive subpopulations of BLA neurons (D) and representative double FISH images (FIG. 5E). Yellow circles=co-expressing neurons. Scale bars, 50 μm.

FIGS. 6A-6F show experimental design for microendoscopy imaging of the BLA during noxious and innocuous events. (FIG. 6A) Coronal schematic of micro-endoscope implant locations, in mice, used in miniature microscope BLA pain imaging experiments (n=17) in FIGS. 1 and 3. The most dorsal GRIN lens (red) was the only left BLA implant. Scale bar, 300 μm. (FIG. 6B) Coronal section of a mouse expressing AAV2/5-Camk2a-GCaMP6m ˜5 weeks (37 days) post-injection showing healthy, cytoplasmic expression of GCaMP6m (green) within the right BLA along with DAPI DNA staining (blue). Scale bar, 500 μm. (FIG. 6C) Zoomed in coronal section of (B) showing anti-GFP staining (Invitrogen a-GFP rabbit A11122) for GCaMP6m (green) and DAPI DNA staining (blue). Scale bar, 100 μm. (FIG. 6D) Neuron map of active neurons from an uninjured right BLA imaging mouse from FIG. 3. A subset of mice contained miniature microscope fields of view divided into BLA and piriform cortex portions separated by a fibre tract (external capsule) that was darker (due to alack of GCaMP6m expression). We manually selected piriform neurons based on location and differential activity compared to BLA neurons then excluded them from further analysis. Scale bar, 100 μm. (FIG. 6E) Example PCA-ICA neuron extraction outputs from a single mouse (same as in FIGS. C-1D) showing accepted and rejected ICA outputs. We manually classified all neurons used in imaging-related aspects of this study based on a variety of parameters, such as the PCA-ICA filter shape, the event triggered movie activity (e.g., whether it confirmed to prior expectation of one-photon neuron morphology and GCaMP activity), location within the imaging field of view (e.g., not within a blood vessel), and the shape of the transient having characteristic GCaMP dynamics. No automated heuristics were used to further remove accepted neurons. “Spatial filters” are the PCA-ICA output filters, “Activity in movie” is a 31×31 pixel square region cropped from the movie around the candidate neuron's centroid location during that candidate neuron's transients (black outlines are “Spatial filter” derived neuron contours), and “Activity traces” shows the mean (black) and per transient (gray) PCA-ICA activity of a candidate neuron from the imaging session. Scale bars, 25 μm. (FIG. 6F) Example neuron map (same animal and imaging session as FIGS. 1C-1D) showing accepted (green) and rejected (red) filters using criterion in (E). Scale bar, 100 μm.

FIGS. 7A-7C. Associate data for FIGS. 1 and 3. Experimental protocol for stimulus delivery during BLA microendoscopy imaging. (FIG. 7A) Experimental timeline and daily imaging protocol for miniature microscope imaged BLA-implanted mice before and after Spared Nerve Injury surgery (SNI). Each session begins with imaging basal neural activity in the BLA without explicit sensory stimulation (Habituation). Next, mice have free access to a lick port delivering adlibitum 10% sucrose (Incentive), which was removed after 15 min. We do not water deprived imaged mice during this or any parts of the protocol. Next, we applied somatosensory stimuli to the hindpaw. The first four stimulus blocks always occur in the same order across days in order to track daily pain behaviors and the development of chronic neuropathic pain. All subsequent stimulus blocks are semi-randomized computer generated sequences within and across days with the conditions that the same stimulus block does not occur twice in sequence, nor does the same daily protocol repeat on any given day. We designed this protocol to be less than 2.5 hr for each animal's imaging session; to give enough stimuli to have sufficient statistical power to identify stimulus-responsive neurons; and to incorporate sufficient “down time” between stimuli, in order to avoid potential photobleaching of imaging area or animal exhaustion. During “Approach” trials either a von Frey filament, water droplet, pin, or noise device was moved toward the animal similar to other trials but with no actual contact or stimulus delivery. Open field imaging sessions consist of 30 min sessions of animals exploring a 2 m diameter circle or 2 m square open field apparatus, see FIG. 11 for additional details and results. (FIG. 7B) Similar experimental timeline and daily imaging session layout as in (FIG. 7A). The protocol was simplified to directly assess relationship between innocuous and nociceptive ensembles before and after nerve injury. (FIG. 7C) Similar experimental timeline and daily imaging session layout as in (FIG. 7A). In addition, these imaging mice had two additional experimental days. “CNO” was a control imaging session in which CNO was injected and 30 min later mice imaged in response to sensory stimuli, see FIGS. 15D-15G for additional details and results. “Aversion” tested responses of BLA neurons to noxious and aversive stimuli, see FIG. 12 for additional details and results. “Habituation” involved mice being habituated to the fear conditioning chambers used in “Aversion” imaging sessions.

FIGS. 8A-8F. (FIG. 8A) A miniature microscope mounted accelerometer (Sparkfun, ADXL335 or ADXL 345) measured quantitative affective escape or reactive measures of animals' responses to various innocuous (0.07-g and 1.4- or 2.0-g von Frey filaments), noxious (55° C. water, 5° C. water, acetone, and 25G pin prick), and control (“Approach/No contact” and noise) stimuli. Mean session acceleration for 2 s after stimulus onset before nerve injury is plotted (n=9 mice). Noxious stimuli all showed significantly more movement than light touch stimuli (One-way ANOVA, Tukey post-hoc). Stars, at least P<0.05. (FIG. 8B) Behavior videos for the same animals as in (FIG. 8A) were manually scored to identify whether animals exhibited reflexive, whole-body, or head-jerk responses after stimulus application (note: this was not a measure of nocifensive paw withdrawal responses). Similar to (FIG. 8A), noxious stimuli (55° C. water, 5° C. water, acetone, and pin prick) all showed significantly (One-way ANOVA, Tukey post-hoc) greater responses than innocuous stimuli. Stars, at least P<0.05. (FIG. 8C) To validate that both the quantitative and manual behavior measures produced similar results, data from (FIG. 8A) and (FIG. 8B) were combined on a per session basis for each stimulus. Before nerve injury (circles and diamonds), both metrics are positively correlated (Spearman's p=0.72 [normal] and 0.59 [neuropathic and uninjured], p-value <0.001). After nerve injury (squares), light touch stimuli (0.07-g, orange) show increased behavioral responses in both metrics that was not seen in uninjured animals (triangles). Inset, zoomed in section (from dotted regions) to better illustrate differences in noxious stimuli responses. (FIG. 8D) Using the same metric as in (FIG. 8A), the responses to noxious stimuli showed similar onset dynamics while light touch stimuli induced minimal behavioral response. Light touch showed a marked increase in onset, in peak reflexive behavior, and in continued escape dynamics after nerve injury (top row) that was not seen in uninjured mice (bottom row), suggesting the presence of neuropathic hypersensitivity and affective allodynia. Further, noxious pin and mild touch stimuli showed heightened responses immediately post-stimulus delivery, suggesting strong neuropathic hypersensitivity has developed, while the lack of an enhanced affective escape response might indicate a saturation or ceiling effect for this measure. The motion responses to “Approach/No contact”, Noise, 10% sucrose, and background are anticipatory escape behavior, startle response followed by escape behavior, head motion toward lick port, and mean movement during random times in the trial when explicit stimuli are not being given, respectively. Baseline (black horizontal line), threshold for movement (gray horizontal line, see FIGS. 1M and 11G, I analysis), and stimulus onset time (black vertical tick) are indicated. Stars, at least P<0.05. (FIG. 8E) Using data in (FIG. 8D), the mean accelerometer response (2 s window after stimuli, red right axis) and human scored reflexive withdrawal (left axis, cyan, same measure as (B)) in uninjured (grey) and neuropathic (red) animals for all stimuli was calculated. Innocuous stimuli showed a significant increase in activity (p<0.01, Wilcoxon rank-sum with Benjamini-Hochberg multiple comparisons correction) with both measures while noxious pin and mild touch showing hypersensitivity trends in escape acceleration. Stars, at least P<0.05. NA indicates background manually scored behavioral responses were not measured. (FIG. 8F) Human scored reflexive responses for all animals (n=17) in FIG. 3C, see (FIG. 8B) and Supplemental Methods for additional details, showing the responses across days before (blue) and after (red) spared nerve injury. Uninjured mice do not show changes in mild or light touch before (green) and after (grey) undergoing a mock surgery (only anesthesia for equivalent time as injured mice). Light colored lines indicate individual mice's responses to each stimulus across imaging sessions. All figure values mean±SEM unless otherwise noted.

FIGS. 9A-9I. Associate data for FIG. 1. BLA neuron ensembles that are selective and co-active to noxious and aversive stimuli. (FIG. 9A) Responses of individual BLA neurons to various noxious (noxious pin, cold, and heat), innocuous (mild and light touch), control (“Approach/No contact”, noise, and background), and positive valence (10% sucrose) stimuli in an uninjured mouse. Mean response (red), activity during individual stimuli trials (gray), and stimuli onset time (black tick) are indicated. (FIG. 9B) Mean stimulus response (Z-scored L\F/F) across all trials for all right BLA neurons during a single imaging session in an uninjured mouse (n=215 neurons). Neuron identifications (rows) across different stimuli are consistent, demonstrating that some neurons encode multiple different types of noxious and innocuous stimuli, while a separate neuron population uniquely encodes nociception. The first three stimuli (noxious heat, cold, and pin) are considered noxious, the mild and light touch are innocuous, the Approach/No contact is a control for animal anticipation of stimuli, the 10% sucrose is an incentive (positive valence), the noise is a mildly aversive control stimuli, and background is a control showing average response during random trial time points at least 10 seconds away from any defined stimuli. (FIG. 9C) Temporal dynamics of the mean L\F/F of neurons within the nociceptive ensemble for all imaging sessions and mice (n=9 mice, 3-4 sessions each). (FIG. 9D) Mean L\F/F of neurons within the nociceptive ensemble for all imaging sessions and mice (n=9 mice, 3-4 sessions each). Values are an average of two seconds post stimulus as seen in (C). Mean BLA nociceptive ensemble response showed a graded reduction from noxious (55° C. water, 5° C. water or acetone, and pinprick) to innocuous and positive valence stimuli. Inset, BLA stimulus response was significantly modulated by stimulus type (One-way ANOVA, F(8,247)=29.4, p<0.001) as shown by the table of significant values (Tukey post-hoc) colored coded by p-value thresholds reached (colored) or not significant (black). (FIG. 9E) Activation of individual neuron ensembles to specific stimuli in the entire (top half, blue) and nociceptive ensembles (bottom half, red) along the diagonal. The percent of total and nociceptive neuron ensembles co-activated by pairs of stimuli (off-diagonal) showed greater co-activation of hindpaw delivered stimuli compared to noise or 10% sucrose stimuli. (FIG. 9F) Spatial locations of neurons, from a single mouse's imaging session, significantly responsive to various noxious (noxious pin and heat), innocuous (light touch), positive valence (10% sucrose), and approach stimuli. A high degree of overlap was seen between noxious stimuli that was absent when compared to positive valence stimuli. Gray neurons are those unresponsive to specific stimuli indicated in each subpanel and green neurons indicate those overlapped between the two stimuli indicated below the sub-panels. Scale bar, 100 μm. (FIG. 9G) The number of stimuli responsive BLA neurons within select ensembles can vary based on ensemble definition. The BLA nociceptive ensemble (˜24% total neurons) was based on the union of all neurons responsive to nociceptive stimuli, this number was reduced when looking at neurons that respond to nociceptive and no other stimuli (˜6% total neurons) or those that respond to all nociceptive stimuli (4-6%). 3397 [normal] and 7535 [neuropathic and uninjured] neurons from 9 mice with 3-4 [normal] and 5-7 [neuropathic or uninjured] sessions each. (FIG. 9H) Stimuli responsive BLA ensembles from animals run through experimental protocol in FIGS. 7A, 7C are slightly more spatially related than the general population (orange) before and after injury or sham. Centroid locations for 3397 (normal), 3783 (neuropathic), and 3752 (uninjured) neurons were computed from PCA-ICA spatial filters and the Euclidean distance calculated to estimate the cumulative and probability densities, see Supplemental Methods. (FIG. 9I) Same as (H) indicating that stimuli-specific BLA ensembles in animals from experimental protocol FIG. 7B exhibit slightly more spatial relatedness in uninjured and neuropathic states as compared to the general neuronal population (n=2839 [8 mice, 22 sessions] and 3625 [8 mice, 26 sessions] neurons for normal and neuropathic, respectively).

FIGS. 10A-10J. Associate data for FIG. 1. BLA neuron stimulus-responsive ensembles are stable across days. (FIG. 10A) Method for cross-day alignment of BLA neural ensembles using real data from an example mouse. Day −2 and 3 are with respect to nerve injury surgery day. After neurons had been matched (steps 4 and 5), they were associated with a global cell that was then used to analyze their responses across days. See Supplemental Methods for detailed procedures. (FIG. 10B) Example neuron spatial filter maps showing cross-day alignment for two example mice's imaging sessions. Global cells matched across at least 70% of the imaging sessions are coded by a unique color. White arrow points to a neuron active across all aligned days for that animal. Scale bars, 100 μm. (FIG. 10C) Cross-day matched neurons showing similar spatial positioning for three example neurons from the right mouse in (B). Red crosses are neuron centroid locations, see Supplemental Methods for details of calculation. (FIG. 10D) Pairwise centroid Euclidean distances for all imaging sessions across mice (n=17) showing that the vast majority of neurons are >10 μm apart. Inset, zoomed in view showing the absolute number of neuron pairs within 10 μm of one another. Red line indicates 0.01t^{h} percentile. Grey line indicates threshold used to group neurons in (FIG. 10A) into a global cell. (FIG. 10E) Same calculation as in (D) except restricted to neuron-neuron pairs within the same global cell, demonstrating the majority of neuron matches assigned to the same global cell are less than 5 μm apart. Red line is at the same location as the 99.99^{th} percentile in (FIG. 10D) inset. (FIG. 10F) Individual neuron distances from their respective global cell centroid location if they were matched to another neuron on at least one other session (n=13,558 session neurons). (FIG. 10G) Example animal showing all global cells (n=146) that were active during greater than half of that animal's imaging sessions. A subset of neurons (bottom rows) is stimulus responsive to noxious cold (acetone) or heat (55° C. water) across multiple imaging sessions and days to weeks of time. Black sections indicate sessions in which no associated neuron was found for that global cell. (FIG. 10H) Number of cross-day global cells across both neuropathic (n=13 mice, n=2,326 global cells) and uninjured control (n=4 mice, 897 global cells) groups. Colors denote individual animals. These same neurons are used for analysis in (FIGS. 10I-10J). (FIG. 10I) Indicates number of global cells that significantly coded for indicated stimuli (see FIGS. 1 and 9) across either one or more imaging sessions irrespective of temporal distance separating imaging sessions. Grey line is 150 global cells and is common across the neuropathic (top row) and uninjured (bottom row) imaging groups. “Nociceptive ensemble” stimuli refers to a global cell that responded to either noxious pin and/or noxious cold on any given imaging session. (FIG. 10J) To determine how long global cells coded for specific stimuli (color coded), actual imaging session dates were used to calculate the maximum duration a global cell was found to be stimulus responsive. Of the 3,223 global cells matched across two or more imaging sessions, ˜11% (350 global cells) responded to noxious stimuli with at least a week separating their first and final noxious stimuli responses.

FIGS. 11A-11I. Associate data for FIGS. 1 and 3. BLA neural activity is correlated with increased motivated escape behaviors, but not general movement. (FIG. 11A) Both dorsomedial striatum-(DMS) (red, n=9 mice, 13 sessions total) and BLA-implanted (blue, n=9 mice, 3-4 sessions each) animals freely explored either a square (60.96×60.96 cm) or circular (60.96 cm diameter) open field for 20-30 min. For each frame in a trial, we calculated the corresponding Ca event-based population activity, the mean taken over specific velocity bins, and the final curve normalized by the mean velocity between 0 and 0.5 cm/s (threshold for movement). DMS, but not BLA, neuron activity showed a modulation in firing rate with velocity. (FIG. 11B) DMS-(red, n=4 mice, 1 session each) and BLA-implanted (blue, n=9 mice, 3-4 sessions each) animals freely explored an open field as in (A). Both animal speed (top) and population activity (bottom, normalized to 2 to 3 seconds before motion onset) were aligned to onset and offset of motion (see Supplemental Methods). Both groups showed similar movement initiation and termination behavior but only the DMS's neuron activity was modulated by start and stop of movement. (FIG. 11C) Example centroid positions of DMS—(left) and BLA-implanted (right) mice during free exploration in their respective open field setups. Early (green) and late (red) session times indicate continuous sampling of the environments throughout the session. Scale bars, 10 cm. (FIG. 11D) Cumulative distance traveled by DMS-(n=9, 1 session each, red) and BLA-implanted (n=9, 3-4 sessions each, blue) mice as run in (FIG. 11A). (FIG. 11E) Mean session velocity for both DMS-(n=9, 1 session each, red) and BLA-implanted (n=9, 3-4 sessions each, blue) mice. (FIG. 11F) Unlike during general movement in (A), increasingly vigorous responses to sensory (noxious cold, heat, and pin and mild and light touch) or “Approach/No contact” stimuli modulated population BLA activity (Spearman's p=[0.21, 0.18], p=[<0.001, <0.001] for [no injury, injury] cases). Graph shows the means±SEM for population response at various levels of animal movement on a per trial basis as recorded from an accelerometer during pain trials as in FIGS. 1E, 1G, and 1M and protocol in FIGS. 7A, 7C. Neuron activity was normalized to trials with less than 0.01 g acceleration (the same acceleration threshold as used to indicate no response in (FIG. 11G), also see FIG. 8D) within each animal's session across all stimuli. Three stars, P<0.001 (Spearman's). (FIG. 11G) The human manually scored and accelerometer calculated reflexive responses compared to % of all neurons activated by the same set of stimuli as in (FIG. 11F). Per trial responses were pooled across animals and the mean animal session response is plotted. They showed significantly increased responses as a larger fraction of the BLA ensemble was activated. For [normal, post-surgery (both neuropathic and uninjured)] cases human scored Spear-man's p=[0.52, 0.44], p-value=[<0.001, <0.001]: accelerometer, p=[0.40, 0.39], p-value=[<0.001, <0.001]. Error bars, ±SEM. 6815 trials, n=9 mice, 3-4 (normal) and 5-7 (neuropathic/uninjured) sessions each. Three stars, P<0.001 (Spear-man's). (FIGS. 11H to 11I) Same as (FIG. 11F) and (FIG. 11G) above except the population has been restricted to the nociceptive ensemble instead of the total neuron population. Error bars, ±SEM. 6815 trials, n=9 mice, 3-4 (normal) and 5-7 (neuropathic/uninjured) sessions each. Three stars, P<0.001 (Spearman's).

FIGS. 12A-12G. Associate data for FIG. 1. BLA nociceptive ensemble overlaps with, but is distinguishable from, aversive ensembles. (FIG. 12A) To compare BLA neuron responses to noxious and aversive stimuli, we adapted our protocol in FIG. 7C to study the response of animals to noxious (noxious heat, cold, and pin), five commonly used aversive (air puff [to the face], isopentylamine [odor], loud noise [˜85 dB], quinine [bitter taste], and 0.6 mA footshock), and a positive valence (10% sucrose) stimuli. (FIG. 12B) Mean stimulus response across all trials for all BLA neurons during a single imaging session in an uninjured mouse (n=162 neurons). Neuron identifications across different stimuli are consistent, demonstrating that some neurons encode multiple different types of noxious and aversive stimuli, while a separate neuron population uniquely encodes nociception (see FIG. 1J). The first three stimuli (noxious heat, cold, and pin) are considered noxious, the next five are aversive (air puff, isopentylamine, noise, quinine, and footshock), 10% sucrose is positive valence, and background is a negative control showing average response during random trial time points at least 10 seconds away from any defined stimuli. (FIG. 12C) Temporal dynamics of the mean AF/F of neurons within the nociceptive ensemble (cyan) and mean affective escape acceleration (red) for all imaging sessions and mice (n=6 mice, 1 session each). (FIG. 12D) Probability of expecting overlap between two stimuli given total and stimuli responsive number of neurons was calculated using the hyper-geometric distribution (see FIGS. 13C-13E and Supplemental Methods). Lower p-values (red) indicate the given overlap between stimuli was less likely to be due to chance. N=6 mice, 1 session each. Symbols indicate various p-value thresholds using the same values used to color code the diagrams. (FIG. 12E) We constructed naïve Bayes decoders as described in FIG. 13A and applied them to the noxious vs. aversive stimuli experiments (n=6 mice, 1 session each). The procedure, color coding, and symbols are as described in FIG. 13B, and see Supplemental Methods. The decoder tended to incorrectly predict one noxious stimuli as another based on the population activity but not the other aversive stimuli. Air puff and isopentylamine may have a high degree of neural ensemble overlap due to a similar method of stimulus delivery. Shuffled stimuli identities indicate that all trends are eliminated when the decoder was trained with the incorrect stimulus labels. (FIG. 12F) The nociceptive ensemble, as defined in FIG. 1H, show less overlap with consummatory stimuli (10% sucrose and quinine) as compared to those stimuli with one another. (FIG. 12G) Increased response to either noxious or aversive stimuli (as defined in (A)) was predictive of amount of neural activity in the BLA (Spearman's p=0.47, p<0.001). Analysis same as in FIGS. 11F, 11H.

FIGS. 13A-13E. Associate data for FIG. 1. BLA stimuli ensembles overlap and exhibit combinatorial coding of nociceptive information. (FIG. 13A) To test out the specificity of the neuronal ensemble dynamics between stimuli, we constructed a nine-way naïve Bayes decoder. For cross-validation, we split data each round 70:30 between training and test datasets using 2 seconds from each trial. After training the decoder, it was run on the test neuron activity data and the predicted stimuli state compared to the actual stimuli delivered (see (B)). The decoder was run through 50 rounds subsampling different sets of trials for use in training and test datasets. (FIG. 13B) The naïve Bayes decoder constructed in (A) was applied to sessions from FIG. 1 (n=9 mice, 3-4 sessions each). The decoder was then run on neural activity data for a new subset of stimuli and the actual stimuli at those frames compared to those predicted. We then normalized each actual stimulus column by the number of total actual stimuli to allow comparisons of how accurate the decoder was. Better performance (red/orange) occurred on noise and 10% sucrose than on innocuous (light purple) or noxious (blue and dark purple) stimuli. Symbols in the off-diagonal indicate whether prediction of correct stimuli was significantly higher than prediction of that stimuli (Wilcoxon sign-rank, Benjamini-Hochberg). Shuffled matrix indicates that all trends are eliminated when the decoder was trained with the incorrect stimuli labels. (FIG. 13C) How unexpected the overlap was in neurons activated by two stimuli depended on the total number of neurons activated by each stimulus, the amount of co-active neurons, and the total number of neurons. To quantify this, either a numerical (shuffling neuron identities and seeing how often they overlap) or exact analytical (using the hypergeometric distribution) solution can be used (see Supplemental Methods). Circles indicate numbers of neurons with gray circles indicating the total population. Number of stimuli activated neurons (red and blue circles) and number of co-activated neurons is the same in columns and rows, respectively. The hypergeometric distribution p-values are shown below each example of stimuli population overlaps. (FIG. 13D) To validate the use of the hypergeometric distribution, we ran 1,000 rounds of 1,000,000 shuffles for the estimated numerical distribution. Using the same number of total neurons, stimuli #1 responsive neurons, stimuli #2 responsive neuron, and overlap neurons, we also calculated the p-values, mean, and standard deviation using the hypergeometric distribution. For the mean and standard deviation, the numerical and analytical solutions were not significantly different (Wilcoxon sign rank, p=0.68 and 0.37 for mean and standard deviation). Paired difference in predicted mean were small (bottom right histogram), likely owing to precision error in numerical calculations since only 1 million shuffles were used. For the p-values, we found a high degree of agreement (95.6%) between overlap identified as significant by numerical and analytical methods (e.g., unexpected given the input parameters). (FIG. 13E) Probability of expecting overlap between two stimuli given total and stimuli responsive number of neurons was calculated using the hyper-geometric distribution, see (FIG. 13C-13D) and Supplemental Methods. Lower p-values (red) indicate the given overlap between stimuli was less likely to be due to chance. All the noxious stimuli are significantly unexpected in the amount of overlap with one another (top left). N=9 mice, 3-4 sessions each. Symbols indicate various p-value thresholds using the same values used to color code the diagrams.

FIGS. 14A-14G. Associate data for FIG. 2. Chemogenetic manipulation of the BLA nociceptive ensemble. (FIG. 14A) Experimental timeline for the noci-TRAP and nociceptive pin prick c-FOS immunohistochemical protocols. (FIG. 14B) Anterior to posterior BLA quantification of noci-TRAP^{eYFP} and c-FOS. (FIG. 14C) Representative image and quantification of anterior BLA noci-TRAP^{eYFP} neurons that were re-activated (c-FOS+) following a second pin prick stimulation 7 days later. (FIG. 14D) Experimental timeline for noci-TRAP for hM4-mCherry expression and subsequent behavioral testing. (FIG. 14E) Representative image of noci-TRAP neurons filled with eYFP. Note the highly branched architecture. (FIG. 14F) Representative image of precise expression of hM4-mCherry in the BLA but not in the neighboring central amygdala nucleus (CeA). Expanded view of same image as in FIG. 2B. Coordinates and structure demarcations from the mouse brain atlas of the Allen Institute for Brain Science. (FIG. 14G) Anatomical maps displaying the area of hM4-mCherry expression across the anterior-posterior amygdala in noci-TRAP mice. The AAV-hSyn-DIO-mCherry was injected at the A-P coordinate, −1.20 mm. On every brain slice illustration each red overlay shows the approximate medial-lateral spread of hM4-mCherry expressing neurons for an individual noci-TRAP mouse with a successful on-target TRAP (i.e., only BLA neurons were TRAP'd); the A-P spread for each mouse is illustrated across the different coordinate brain slices (n=7 mice). The blue overlays on each brain slice indicate mice with off-target TRAP outside the BLA, primarily in the CeA. Based on these criteria n=7 mice were excluded from the data set in FIG. 2.

FIGS. 15A-15G. Associate data for FIG. 2. Chemogenetic separation of affective-motivational pain behaviors from reflexive responses. (FIG. 15A) Illustrative progression of pain behaviors, from the immediate reflexes to the temporally delayed affective-motivational behaviors, following delivery of a noxious stimulus. (FIG. 15B) Same mice as in FIGS. 2F and 2G are shown, but we display here the separate scores for subcategories of affective-motivational behaviors: attending (top rows) or escape behavior (bottom rows). N=14 mice/group. (FIG. 15C) Lack of effect of clozapine-N-ox-ide (CNO, 10 mg/kg) on reflexive (left green y-axis) or affective-motivational (right red y-axis) behaviors in control mice expressing eYFP in the BLA nociceptive ensemble (AAVDJ-Ef1α-DIO-eYFP). N=6 mice/group. (FIG. 15D) To compare BLA neuron responses before and after CNO application, we adapted our protocol in FIG. 7C to study the responses of animals 30 min after CNO (10 mg/kg) injection. Mice were then tested on a simplified version of protocol in FIG. 7C. (FIG. 15E) Lack of behavioral effect of CNO (F(1,60)=0.016, p=0.90, One-way ANOVA) and absence of statistical interaction between CNO and stimulus (F(2,60)=0.14, p=0.87, Two-way ANOVA). N=6 mice, 1 session each, n.s.=no significant difference before and after CNO injection (Wilcoxon rank-sum). (FIG. 15F) Mean stimulus response across all trials for all BLA neurons during a single imaging session in an uninjured mouse (n=104 neurons). Neuron identification across different stimuli is consistent, demonstrating that some neurons encode different types of stimuli. Stimuli are the same as in FIG. 9B. (FIG. 15G) Mean neural response to various stimuli on sessions where we did not (Day 35, 42) or did (CNO) inject mice with CNO (10 mg/kg). CNO did not alter neural responses, F(1,178)=1.002, p=0.318, One-way ANOVA pooled over groups and stimuli. n.s.=no significant difference before and after CNO injection (Wilcoxon rank-sum). Stars, P<0.05. Overlaid small dots and lines are individual subjects. Large dots represent group mean responses and error bars show ±SEM.

FIGS. 16A-16D. Associate data for FIG. 2. Thermal track occupancy. (FIG. 16A) Individual trial and group average occupancy paths for noci-TRAP^{hM4} mice treated with saline. (FIG. 16B) Individual trial and group average occupancy paths for noci-TRAP^{hM4} mice treated with CNO. (FIG. 16C and FIG. 16D) Cumulative occupancy inside the (C) noxious cold or (D) noxious hot zones for noci-TRAP^{hM4} mice treated with either saline or CNO. Stars, P<0.05, Kolmogorov-Smirnov test.

FIG. 17A-17O. Associate data for FIG. 2. Optogenetic activation of nociceptors elicits pain affective-motivational behaviors that require the BLA nociceptive ensemble. (FIG. 17A) Optogenetic nociceptive TRAP of hM4-mCherry in the BLA (o-TRAP^{hM4}). Cre-dependent AAV5-hSyn-DIO-hM4-mCherry was stereotaxically injected into the bilateral BLA of TRAP mice, followed by a spinal intrathecal injection of AAV6-hSyn-ChR2(H134R)-eYFP to infect peripheral dorsal root ganglion nociceptors. After 3-4 weeks, transdermal blue light was applied to the left hindpaw to activate the light-sensitive cation channel ChR2 in nociceptors (TRAP stimulus), which was followed by injection of 4-OHT 60 min later to induce DNA recombination and expression of hM4-mCherry in the BLA. Behavioral tests take place 3-5 weeks later. (FIG. 17B) Expression of ChR2-eYFP in peripheral CGRP+ nociceptors of dorsal root ganglia. (FIG. 17C) ChR2-eYFP was trafficked to the cutaneous terminals of CGRP+ nociceptors. These free nerve endings innervate the epidermis of the glabrous skin where they can be activated by transdermal 450 nm light. (FIG. 17D) The central terminals of ChR2-eYFP+ nociceptors innervate the substantia gelatinosa of the spinal cord dorsal horn. Repeated transdermal light stimulations (1 s; 2-3 mW/mm²) induce FOS expression in dorsal horn neurons within the terminal fields of ChR2-eYFP+ nociceptors, indicating transmission of nociceptive information to the CNS. (FIG. 17E) Transdermal optogenetic nociception drives FOS expression in the BLA. (FIG. 17F to FIG. 17J) Additional immunohistochemical characterization of the peripheral afferent populations expressing viral ChR2-eYFP. ChR2-eYFP was predominately expressed in peptidergic CGRP+ nociceptors, and mostly excluded from the IB4+/Ret+ non-peptidergic nociceptor populations. (FIG. 17K) Optogenetic nociception elicits pain affective-motivational behaviors, such as attending and escape, similar to natural noxious stimuli. There was no effect of transdermal light on behavioral responses before expression of ChR2 or in mice expressing GFP in nociceptors. (FIG. 17L) Quantification of FOS expression in the BLA induced by optogenetic nociception. (FIG. 17M) Quantification of hM4-mCherry expression in the BLA following o-TRAP. (FIG. 17N) CNO-mediated silencing of the BLA nociceptive ensemble reduces attending and escape behaviors in response to noxious transdermal light stimuli. (FIG. 17O) (Left) Optical real-time place escape avoidance task in which one chamber floor was illuminated by a LED array red or blue light. (Right) Control mice expressing GFP in nociceptors show no preference between either chamber. ChR2-eYFP+ nociceptor mice given a saline injection significantly avoid the blue light chamber. CNO treatment (10 mg/kg) in a separate group of ChR2-eYFP+ nociceptor mice eliminated the aversion to the noxious blue light chamber. FIGS. 17B-17J, n=3 mice. FIGS. 17K-17N, n=5 mice/group; FIGS. 17K, 17N, RM Two-way ANAOVA+Bonferroni; FIGS. 17L,17M, Student's t Test. FIG. 17O, n=4 mice/group; One-way ANAOVA+Bonferroni. Stars, P<0.05 for all panels. Error bars, ±SEM. In (FIG. 17E), scale bar, 100 μm. All other scale bars, 50 μm. ChR2, Chan-nelrhodopsin2; eYFP, enhanced Yellow Fluorescent Protein; CGRP, Calcitonin Gene-Related Peptide; NF200, Neurofilament 200; TRKB, Tropomyosin receptor kinase B; 1B4, Isolectin B4; RET, Proto-oncogene tyrosine-protein kinase receptor Ret.

FIGS. 18A-18C. Associate data for FIG. 2. Anxiety-like and incentive motivational behavior. (FIG. 18A) Temporal exploration paths on an elevated plus maze. Group mean occupancy path traces for noci-TRAP^{hM4} mice given either saline or CNO (10 mg/kg). (FIG. 18B) No effect of CNO-mediated silencing of the BLA nociceptive ensemble on the total distance traveled, or average velocity of mice in the Elevated Plus Maze. Overlaid dots are individual subjects. Error bars, ±SEM. (FIG. 18C) Daily incentive approach behavior during exposure to an unconditioned sucrose-water preference task. Left: Daily cumulative lick bouts at either a 10% sucrose port or a water port over a 7-day trail period. noci-TRAP^{hM4} mice were given either saline or CNO 60 min prior to the start of the daily trial. Right: Daily consumption of water and sucrose displayed as a % sucrose preference over water. Boxplots display the 1^{st}, 2^{nd}, and 3^{rd} quartiles with whiskers indicating 1.5*IQR. Stars, P<0.05. Note that on the first and second days (Sessions 1 and 2) of conditioning the noci-TRAPhM4 mice treated with CNO displayed a significant sucrose preference over mice treated with saline.

FIGS. 19A-19F. Associate data for FIG. 3. BLA spontaneous activity and neuron ensemble activity and overlap after nerve injury. (FIG. 19A) There were no significant spontaneous activity increases or decreases in the nociceptive or non-nociceptive neuron populations after spared nerve injury (p=0.11 [nociceptive] and 0.58 [non-nociceptive], Wilxocon rank-sum, n=13 mice) or in uninjured mice (p=0.82 [nociceptive] and 0.13 [non-nociceptive], Wilxocon rank-sum, n=4 mice). Spontaneous BLA neuron activity was measured during a 10 to 15 min habituation period that either took place in a small chamber separate from pain experiments (n=8 mice) or within the test chamber itself (n=9 mice). We calculated the BLA Ca²⁺ transient rate (see Supplemental Methods) for each session and the rates for each animal normalized by the mean session activity across all sessions before spared nerve injury or sham surgery. Individual gray lines indicate mean per animal changes in BLA Ca²⁺ transient rate before and after spared nerve injury or sham surgery. (FIG. 19B) Same as in (A), showing the spontaneous BLA Ca²⁺ transient activity across imaging sessions before and after spared nerve injury (red) or sham surgery (gray). For post-SNI days 28, 35, and 42, n=9 mice from experimental protocol in FIGS. 7A, 7C are included compared to a combination of all mice (n=17) across both pre-SNI sessions and days 3-21 post-SNI. (FIG. 19C) Mean session population L\F/F activity for neurons within the noxious ensemble (either 5° C. [n=2 mice] or acetone [n=15 mice] and pin prick) before and after spared nerve injury normalized to noxious cold and pin L\F/F on a per animal session basis. Innocuous (mild and light touch) stimuli showed a significant increase in activity specifically in nerve injured animals (Wilcoxon rank-sum, Benjamini-Hochberg corrected). Individual gray lines indicate mean per animal changes in L\F/F response before and after injury or sham surgery. Stars, P<0.001. (FIG. 19D) Percentage of significantly responding neurons to noxious and touch stimuli before and after nerve injury or sham surgery. Light touch stimuli show a significant increase specifically in injured animals. Stars, P<0.001, Wilcoxon rank-sum test. Gray lines indicate individual animal mean % of neurons activated. (FIG. 19E) Light touch neural ensemble had a more unexpected overlap with noxious cold (5° C. water or acetone) ensemble after spare nerve injury (n=13) but not in uninjured control animals (n=4). The mean overlap between pairs of stimuli ensembles is indicated by boxplots while the green dot indicates the median±[1^{st} and 3^{rd} quartiles] expected overlap between stimuli (calculated from hypergeometric distribution, see FIGS. 13C-13E and Supplemental Methods). Gray lines are individual animals before and after spared nerve injury or sham surgery. Significant change in overlap regardless of expectedness is indicated by bar connecting “no injury” to SNI while white star within boxplot indicates significant difference from expected mean overlap. Significance calculated using Wilcoxon rank-sum with Benjamini-Hochberg multiple-comparisons correction. (FIG. 19F) Similar plot as in FIG. 3H. Correlation between % of nociceptive ensemble activated and escape acceleration per imaging session (light colored points) and across animal groups and conditions (dark, larger points) show significant correlation (Spear-man's p=0.60 [Normal], 0.49 [Neuropathic] and 0.54 [Uninjured]). Three stars, P<0.001.

FIGS. 20A-20F. Associate data for FIG. 4. Strategy to manipulate BLA ensembles involved in chronic pain affect. (FIG. 20A) Experimental and behavioral testing timeline for light touch-TRAP of DREADD(hM4)-mCherry in neuropathic mice with mechanical allodynia. (FIGS. 20B and 20C) Nociceptive mechanical sensitivity before and after sciatic nerve injury, quantified by (FIG. 20B) withdrawal frequency to intensifying von Frey filament stimulations and by (FIG. 20C) mechanical thresholds using the Up-Down method. (FIGS. 20D and 20E) Pain affective-motivational hyperalgesia in response to (FIG. 20D) pin prick and (FIG. 20E) acetone drop. (FIG. 20F) Anatomical maps displaying the area of hM4-mCherry expression across the anterior-posterior amygdala in light touch-TRAP mice. The AAV-hSyn-DIO-mCherry was injected at the A-P coordinate, −1.20 mm. On every brain slice illustration, each red overlay shows the approximate medial-lateral spread of hM4-mCherry expressing neurons for an individual light touch-TRAP mouse with a successful on-target TRAP (i.e., only BLA neurons were TRAP'd); the A-P spread for each mouse is illustrated across the different coordinate brain slices (n=7 mice). The blue overlays on each brain slice indicate mice with off-target TRAP outside the BLA, primarily in the CeA. Based on this criteria, n=5 mice were excluded from the data set in FIG. 4. The underlying histogram displays the means±SEM. quantification of light touch-TRAP^{hM4} neurons along the anterior-posterior axis of the BLA (n=7 on-target TRAP mice).

FIGS. 21A-21C. Chemogenetic reduction in chronic pain affect. (FIG. 21A to FIG. 21B) Same mice as in FIG. 4E, but displaying the separate scores for different subcategories of af-fective-motivational behaviors; attending (B, D) or escape (C, E), in mice with nerve injury (B, C) or without injury (D, E). (FIG. 21C) Lack of effect of CNO (10 mg/kg) on reflex behaviors elicited by stimulation with von Frey filaments (left panel) or on affective-motivational behaviors in response to an acetone drop (right panel) in control neuropathic mice (i.e., not expressing hM4-mCherry in the BLA). Morphine served as a positive control and reduced both reflexive and affective-motivational pain behaviors. Stars, P<0.05. Overlaid lines or small dots are individual subjects. Large dots represent group mean responses and error bars show ±SEM.

### DETAILED DESCRIPTION

The inventors have identified an ensemble of neurons in the basolateral amygdala (BLA) that encodes nociceptive information across pain modalities, including pain evoked by noxious thermal and mechanical stimuli. Methods are provided for screening candidate agents for inhibition of neural activity of neurons within the BLA nociceptive ensemble. Screening assays further include determining the effectiveness of candidate agents in alleviating pain and reducing aversive pain avoidance behavior.

Before the BLA nociceptive ensemble and methods of screening candidate agents for effectiveness in inhibiting neural activity within the BLA nociceptive ensemble and alleviating pain and/or reducing aversive pain avoidance behavior are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited.

It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As used herein the singular forms “a”, “an”, and “the” include plural referents unless the context clearly dictates otherwise. Thus, for example, reference to “an agent” includes a plurality of such agents and reference to “the drug” includes reference to one or more drugs and equivalents thereof (e.g., therapeutics, medicines, medicaments), known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “about,” particularly in reference to a given quantity, is meant to encompass deviations of plus or minus five percent.

By “pathological pain” is meant any pain resulting from a pathology, such as from functional disturbances and/or pathological changes, lesions, burns and the like. One form of pathological pain is “neuropathic pain” which is pain thought to initially result from nerve damage but extended or exacerbated by other mechanisms including glial cell activation. Examples of pathological pain include, but are not limited to, thermal or mechanical hyperalgesia, thermal or mechanical allodynia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, and the like.

“Hyperalgesia” refers to an abnormally increased sensitivity to pain, including pain that results from excessive sensitivity to stimuli. Hyperalgesia can result from damage to nociceptors or nerves. Primary hyperalgesia refers to pain sensitivity that occurs in damaged tissues. Secondary hyperalgesia refers to pain sensitivity that occurs in undamaged tissue surrounding damaged tissue. Examples of hyperalgesia include, without limitation, thermal hyperalgesia (i.e., hypersensitivity to cold or heat) and opioid-induced hyperalgesia (e.g., hypersensitivity to pain as a result of long-term opioid use such as caused by treatment of chronic pain).

“Hypalgesia” or “hypoalgesia” refers to decreased sensitivity to pain.

“Allodynia” means pain that results from a normally non-painful, non-noxious stimulus to the skin or body surface. Examples of allodynia include, but are not limited to, thermal (hot or cold) allodynia (e.g., pain from normally mild temperatures), tactile or mechanical allodynia (e.g., static mechanical allodynia (pain triggered by pressure), punctate mechanical allodynia (pain when touched), or dynamic mechanical allodynia (pain in response to stroking or brushing)), movement allodynia (pain triggered by normal movement of joints or muscles), and the like.

“Nociception” is defined herein as pain sense. “Nociceptor” herein refers to a structure that mediates nociception. The nociception may be the result of a physical stimulus, such as, a mechanical, electrical, thermal, or a chemical stimulus. Nociceptors are present in virtually all tissues of the body.

“Analgesia” is defined herein as the relief of pain without the loss of consciousness. An “analgesic” is an agent or drug useful for relieving pain, again, without the loss of consciousness.

The term “administering” is intended to include routes of administration which allow the agent to perform its intended function (e.g., modulating pain perception and/or modulating neural activity of one or more neurons in the BLA nociceptive ensemble). Examples of routes of administration which can be used include injection (intraneural, intracranial, intracerebral, subcutaneous, intravenous, parenteral, intramuscular, intraperitoneal, intrathecal, intraspinal, etc.), oral, intranasal, inhalation, and transdermal. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. Further, the agent may be coadministered with a pharmaceutically acceptable carrier. The agent also may be administered as a prodrug, which is converted to its active form in vivo.

As used here, the term “modulating pain” refers to the modulation (e.g., inhibition or diminishment) of pain or the perception of pain in a given subject and includes absence from pain sensations as well as states of reduced or absent sensitivity to pain stimuli.

As used here, the term “modulating the activity” of a given target cell (e.g., neuron) refers to changing the activity level of a cell function. For example, altering the activity of a target neuron may include changing the membrane potential of a neuron, wherein the membrane potential of a neuron is important for its function (e.g., action potential firing). In some cases, the activity of the neuron is altered such that the membrane potential is increased (e.g., hyperpolarized). In some cases, the activity of the neuron is altered such that the membrane potential is decreased below a threshold potential, resulting in an action potential (e.g., depolarized).

The terms “pharmacologically effective amount” or “therapeutically effective amount” of a composition or agent, as provided herein, refer to a nontoxic but sufficient amount of the composition or agent to provide the desired response, such as a reduction or reversal of neuropathic pain, pathological pain, or chronic pain and/or reducing or eliminating aversive pain avoidance behavior. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Treatment” or “treating” pain includes: (1) preventing pain, i.e., causing pain not to develop or to occur with less intensity in a subject that may be exposed to or predisposed to pain but does not yet experience or display pain, (2) inhibiting pain, i.e., arresting the development or reversing pain, (3) relieving pain, i.e., decreasing the amount of pain experienced by the subject, or (4) reducing or eliminating pain avoidance behavior.

By “treating existing pain” is meant attenuating, relieving or reversing pathological pain in a subject that has been experiencing pain for at least 24 hours, such as for 24-96 hours or more, such as at least 25, 30, 35, 40, 45, 48, 50, 55, 65, 72, 80, 90, 96, or 100 or more hours. The term also intends treating pain that has been occurring long-term, such as for weeks, months or even years.

As used herein, the term “determining” refers to both quantitative and qualitative determinations and as such, the term “determining” is used interchangeably herein with “assaying,” “measuring,” and the like.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Substantially purified” generally refers to isolation of a substance (e.g., compound, polynucleotide, protein, polypeptide, antibody, aptamer, receptor ligand) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

By “subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like.

The term “antibody” encompasses polyclonal antibodies, monoclonal antibodies as well as hybrid antibodies, altered antibodies, chimeric antibodies, and humanized antibodies. The term antibody includes: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Nat Acad SciUSA 69:2659-2662; and Ehrlich et al. (1980) Biochem 19:4091-4096); single-chain Fᵥ molecules (scFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

The phrase “specifically (or selectively) binds” with reference to binding of an antibody to an antigen refers to a binding reaction that is determinative of the presence of the antigen in a heterogeneous population of proteins and other biologics. Thus, under designated immunoassay conditions, the specified antibodies bind to a particular antigen at least two times the background and do not substantially bind in a significant amount to other antigens present in the sample. Specific binding to an antigen under such conditions may require an antibody that is selected for its specificity for a particular antigen. For example, antibodies raised to an antigen from specific species such as rat, mouse, or human can be selected to obtain only those antibodies that are specifically immunoreactive with the antigen and not with other proteins, except for polymorphic variants and alleles. This selection may be achieved by subtracting out antibodies that cross-react with molecules from other species. A variety of immunoassay formats may be used to select antibodies specifically immunoreactive with a particular antigen. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

Screening for Agents that Reduce Pain Perception or Aversive Pain Avoidance Behavior

The inventors have discovered a core set of neurons in the basolateral amygdala (i.e., the BLA nociceptive ensemble) that function in the perception of acute and chronic pain. Additionally, increasing activation of the BLA nociceptive ensemble correlates with increased pain affective-motivational behavior. Silencing the BLA neural ensemble alleviates pain affective-motivational behavior without altering the detection of noxious stimuli. Accordingly, screening methods for identifying candidate agents that inhibit neural activity of the BLA neural ensemble are provided.

In some embodiments, the screening method comprises contacting one or more neurons of the BLA nociceptive ensemble in the brain of a subject with a candidate agent and monitoring pain perception or pain avoidance behavior of the subject. A subset of neurons of the BLA nociceptive ensemble or all of the neurons of the BLA nociceptive ensemble may be contacted with the agent.

In some embodiments, a candidate agent is screened in a test subject. The test subject may be any subject having a BLA neural ensemble that is capable of perceiving pain. Various methods are known in the art for measuring the perception of pain by a subject. Test subjects may be human or non-human. Non-human test subjects may include, for example, mammals, including, without limitation, carnivores (e.g., dogs and cats), rodents (e.g., mice, guinea pigs and rats), and primates (e.g., chimpanzees and monkeys).

A variety of screening methods may be used for assessing whether an agent relieves pain and/or reduces pain affective-motivational behavior including sensory perception of pain, pain avoidance behavior, hyperalgesia, and allodynia. Exemplary screening methods include, without limitation, stimulus-evoked behavioral tests such as a mechanical withdrawal test, an electronic Von Frey test, a manual Von Frey test, a Randall-Selitto test, a Hargreaves test, a hot plate test, a cold plate test, a thermal probe test, an acetone evaporation test, cold plantar test, and a temperature preference test; and non-stimulus-evoked behavioral tests such as a grimace scale test, weight bearing and gait analysis, locomotive activity test (e.g., still, walking, trotting, running, distance traveled, velocity, eating/drinking and foraging behavior frequencies), and burrowing behavior test. See, e.g., Deuis et al. (2017) Front Mol Neurosci. 10:284, Yuan et al. (2016) Adv Exp Med Biol. 904:1-22, Navratilova et al. (2013) Ann N Y Acad Sci. 1282:1-11; herein incorporated by reference.

Pain induced by mechanical stimuli may include mechanical hyperalgesia or allodynia, which can be subdivided into dynamic (triggered by brushing), punctate (triggered by touch) and static (triggered by pressure) subtypes of hyperalgesia or allodynia. Testing for dynamic mechanical allodynia and hyperalgesia may include, for example, brushing the skin of a subject with a cotton ball or paintbrush. Punctate mechanical allodynia and hyperalgesia can be tested, for example, with a pinprick or von Frey filaments of varying forces (0.08-2940 mN). Static hyperalgesia can be tested, for example, by applying pressure to the skin or underlying tissue by pressing a finger or using a pressure algometer.

Pain induced by heat or cold stimuli may include thermal hyperalgesia or allodynia. Thermal hyperalgesia or allodynia may be tested, for example, by applying a metal probe to the skin that increases or decreases in temperature to determine a threshold temperature at which pain is experienced. Pain induced by heat is typically experienced at temperatures of 42-48° C., and pain induced by cold is typically experienced at temperatures of 23.7-1.5° C.

For testing of pain in animals, pain is inferred from “pain-like” behaviors, such as withdrawal from a nociceptive stimulus. An animal is considered to have allodynia if the animal withdraws from an innocuous stimulus that does not normally evoke a withdrawal response. An animal is considered to have hyperalgesia if an animal withdraws with an exaggerated response to a stimulus that does normally evoke a withdrawal response. Responses of animals to mechanical stimuli can be tested using a manual or electronic Von Frey test or the Randall Selitto test. Responses of animals to heat stimuli can be tested, for example, using the tail flick test, the Hargreaves test, a hot plate test, or a thermal probe test. Responses of animals to cold stimuli can be tested, for example, using a cold plate test, an acetone evaporation test, a cold plantar assay. Thermal hyperalgesia or allodynia can be tested in animals for example by using a temperature preference test. For example, an animal is allowed to choose between two adjacent areas maintained at different temperatures or a preferred position along a continuous temperature gradient (either in linear or circular form).

A grimace scales test can be used to score the subjective intensity of pain based on facial expressions of a subject. In rodents (e.g., rats and mice), facial features can be scored, including orbital tightening, nose/cheek bulge or flattening, ear position, and whisker position. Burrowing, which is a self-motivated behavior, can also be used as a measure of spontaneous or non-stimulus evoked nociception in mice and rats. Gait and weight bearing of rodents also can be analyzed as an indicator of nociception.

Other behavior that can be analyzed in test subjects include locomotive activity (still, walking, trotting, running), distance traveled, velocity, grooming, posture, eating/drinking and foraging. The frequencies of these behaviors in animal models of pain are compared to control states to determine if an agent alleviates pain or pain-motivated behavior.

A variety of different test agents may be screened for their effects on inhibition of neural activity of the of the BLA nociceptive ensemble and pain perception or pain affective-motivational behavior. Candidate agents encompass numerous chemical classes, e.g., small organic compounds having a molecular weight of more than 50 daltons and less than about 10,000 daltons, less than about 5,000 daltons, or less than about 2,500 daltons. Test agents can comprise functional groups necessary for structural interaction with proteins, e.g., hydrogen bonding, and can include at least an amine, carbonyl, hydroxyl or carboxyl group, or at least two of the functional chemical groups. The test agents can comprise cyclical carbon or heterocyclic structures and/or aromatic or polyaromatic structures substituted with one or more of the above functional groups. Test agents are also found among biomolecules including peptides, saccharides, fatty acids, steroids, purines, pyrimidines, derivatives, structural analogs or combinations thereof.

Test agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In some embodiments, test agents are synthetic compounds. A number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the test agents are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In some embodiments, the test agents are organic moieties. In this embodiment, test agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

In some embodiments test agents are assessed for any cytotoxic activity it may exhibit toward a living eukaryotic cell, using well-known assays, such as trypan blue dye exclusion, an MTT (3-(4,5-dimethylthiazol-2-yl)-2,5-diphenyl-2 H-tetrazolium bromide) assay, and the like. Agents that do not exhibit significant cytotoxic activity are considered candidate agents.

In some embodiments, the test agent is an antibody that specifically binds to a receptor and alters neural activity of a neuron of the BLA nociceptive ensemble. Any type of antibody may be screened for the ability to inhibit neural activity of a neuron of the BLA nociceptive ensemble by the methods described herein, including polyclonal antibodies, monoclonal antibodies, hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fᵥ molecules (sFv) (see, e.g., Huston et al. (1988) Proc Natl Acad Sci USA 85:5879-5883); nanobodies or single-domain antibodies (sdAb) (see, e.g., Wang et al. (2016) Int J Nanomedicine 11:3287-3303, Vincke et al. (2012) Methods Mol Biol 911:15-26; dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

In other embodiments, the test agent is an aptamer that specifically binds to a receptor and alters neural activity of a neuron of the BLA nociceptive ensemble. Aptamers may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Aptamers Selected by Cell-SELEX for Theranostics (W. Tan, X. Fang eds., Springer, 2015), Cox et al. (2001) Bioorg. Med. Chem. 9(10)2525-2531; Cox et al. (2002) Nucleic Acids Res. 30(20): e108, Kenan et al. (1999) Methods Mol. Biol. 118:217-231; Platella et al. (2016) Biochim. Biophys. Acta Nov 16 pii: S0304-4165(16)30447-0, and Lyu et al. (2016) Theranostics 6(9):1440-1452; herein incorporated by reference in their entireties.

In yet other embodiments, the test agent is an antibody mimetic that specifically binds to a receptor and alters neural activity of a neuron of the BLA nociceptive ensemble. Any type of antibody mimetic may be used, including, but not limited to, affibody molecules (Nygren (2008) FEBS J. 275 (11):2668-2676), affilins (Ebersbach et al. (2007) J. Mol. Biol. 372 (1):172-185), affimers (Johnson et al. (2012) Anal. Chem. 84 (15):6553-6560), affitins (Krehenbrink et al. (2008) J. Mol. Biol. 383 (5):1058-1068), alphabodies (Desmet et al. (2014) Nature Communications 5:5237), anticalins (Skerra (2008) FEBS J. 275 (11)2677-2683), avimers (Silverman et al. (2005) Nat. Biotechnol. 23 (12):1556-1561), darpins (Stumpp et al. (2008) Drug Discov. Today 13 (15-16):695-701), fynomers (Grabulovski et al. (2007) J. Biol. Chem. 282 (5):3196-3204), and monobodies (Koide et al. (2007) Methods Mol. Biol. 352:95-109).

Candidate agents can be detectably labeled by well-known techniques. Detectable labels include, for example, radioactive isotopes, fluorescent labels, chemiluminescent labels, bioluminescent labels and enzyme labels. Such labeled inhibitors can be used to determine cellular uptake efficiency, quantitate binding of inhibitors at target sites, or visualize inhibitor localization.

Assays may further include suitable controls (e.g., untreated with candidate agent or any other analgesic agent). Generally, a plurality of tests is run in parallel with different agent concentrations used on different test subjects to obtain a differential response to the various concentrations. Typically, one of these concentrations serves as a negative control, i.e., at zero concentration or below the level of detection.

### Pharmaceutical Compositions

Agents, identified by the screening methods described herein, as useful for alleviating pain and/or reducing aversive pain-motivated behavior can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the agent, or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the agent (e.g., when contained in a drug delivery system) in the composition will vary depending on a number of factors but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for intraneural, intracerebral, intrathecal, intraspinal, or localized delivery such as by stereotactic injection into the BLA nociceptive ensemble in the brain.

The pharmaceutical preparations herein can also be housed in a syringe, an implantation device, or the like, depending upon the intended mode of delivery and use. Preferably, the compositions comprising the agent are in unit dosage form, meaning an amount of a conjugate or composition of the invention appropriate for a single dose, in a premeasured or pre-packaged form.

The compositions herein may optionally include one or more additional agents, such as analgesics or one or more other drugs for treating pain or other medications. For example, compounded preparations may include at least one candidate agent and one or more other drugs for treating pain, including, without limitation, acetaminophen, nonsteroidal anti-inflammatory drugs (e.g., aspirin, ibuprofen and naproxen), COX-2 inhibitors (e.g., rofecoxib, celecoxib, and etoricoxib), opioids (e.g., morphine, codeine, oxycodone, hydrocodone, dihydromorphine, and pethidine), gabapentin, memantine, pregabalin, cannabinoids, tramadol, lamotrigine, carbamazepine, duloxetine, milnacipran, tricyclic antidepressants (e.g., amitriptyline, nortriptyline, and desipramine), and serotonin-norepinephrine reuptake inhibitors (e.g., duloxetine, venlafaxine, and milnacipran).

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

An Amygdalar Neural Ensemble that Encodes the Unpleasantness of Pain

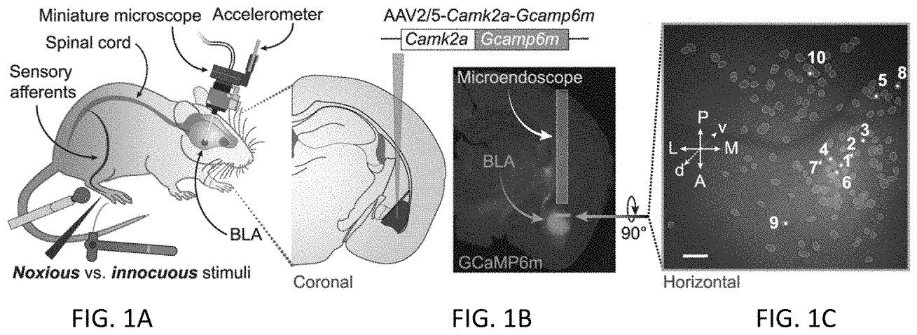

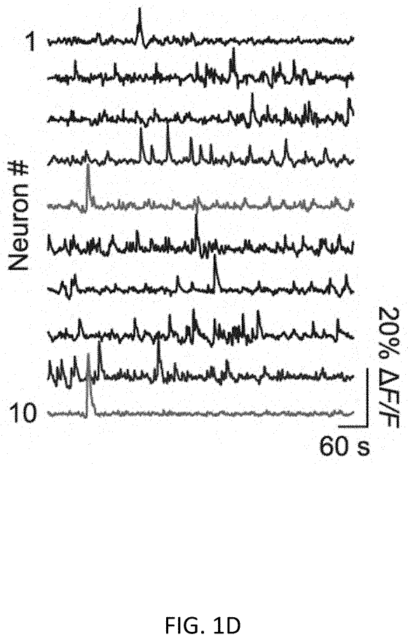

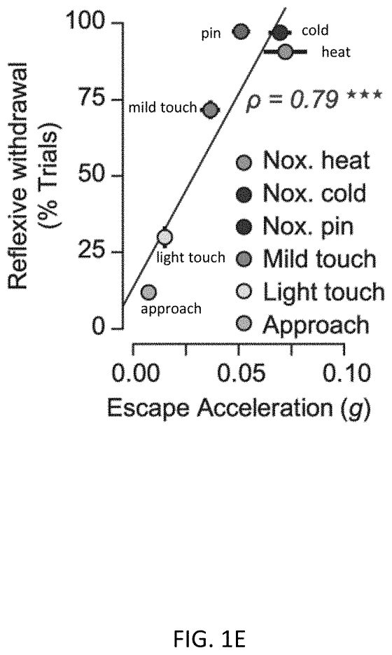

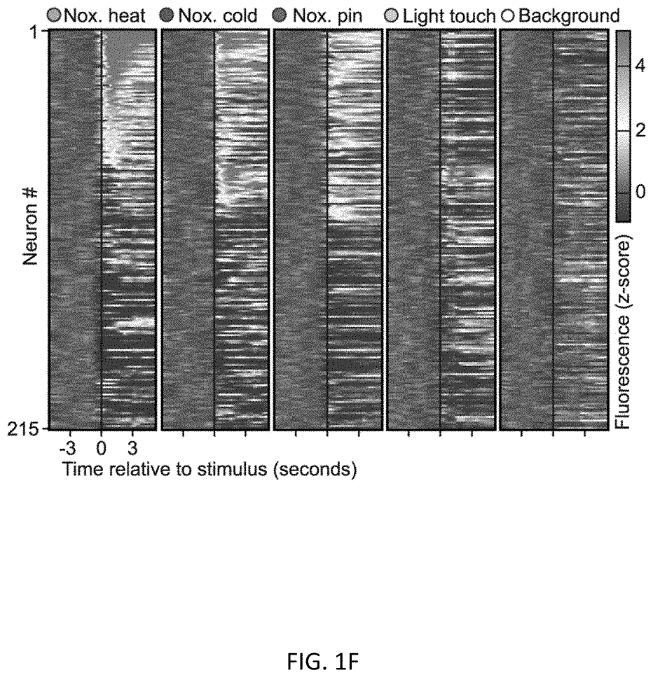

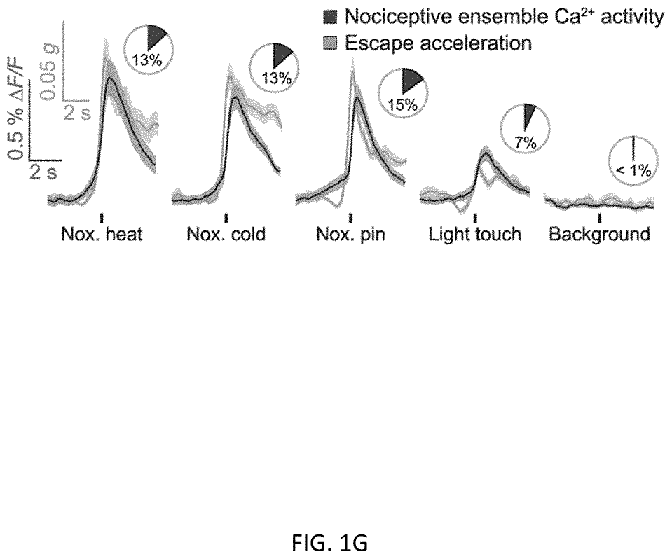

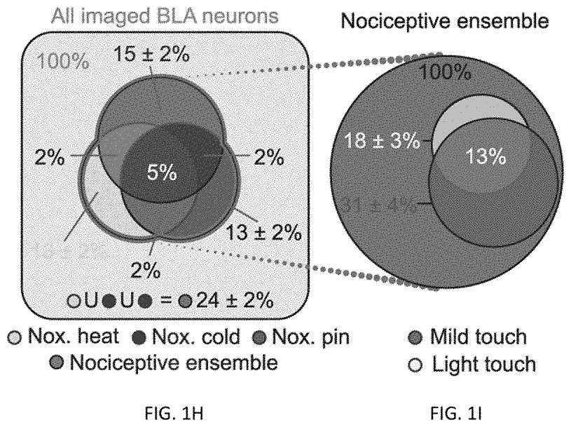

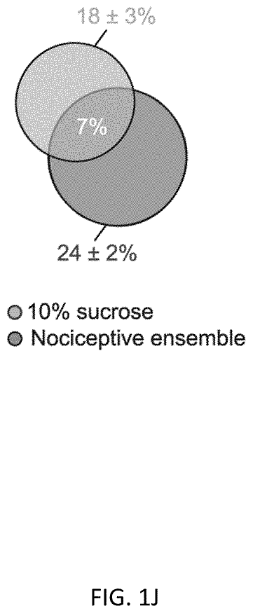

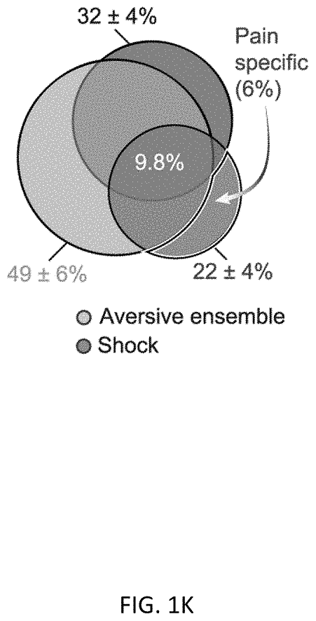

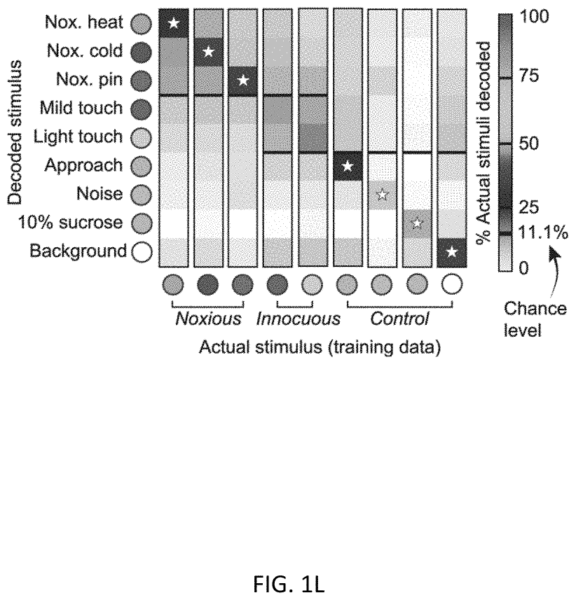

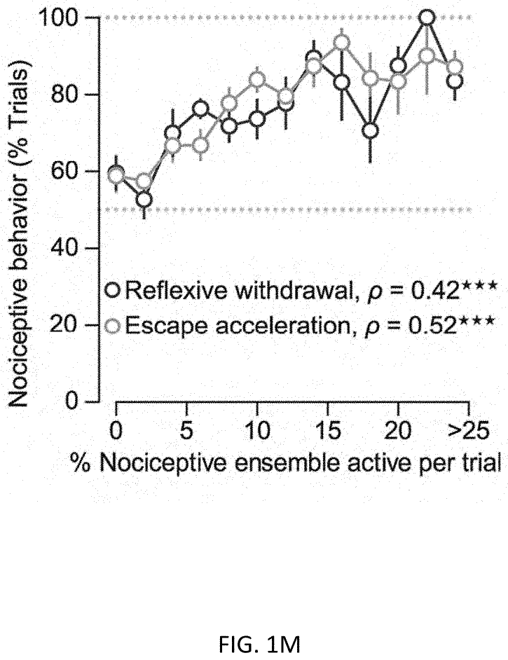

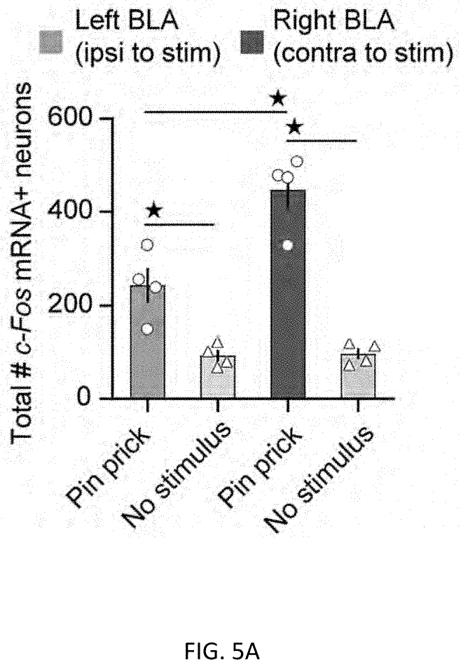

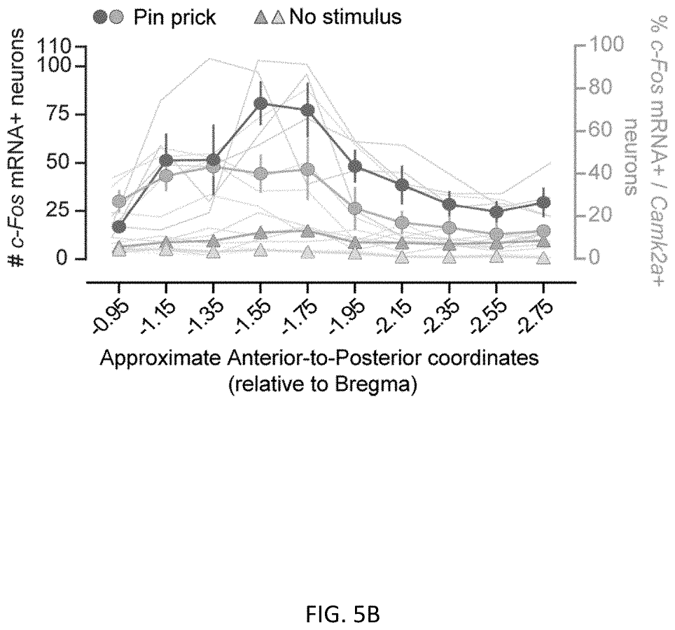

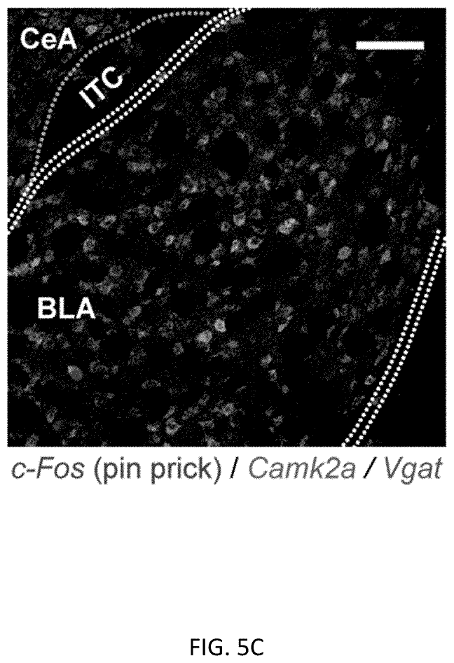

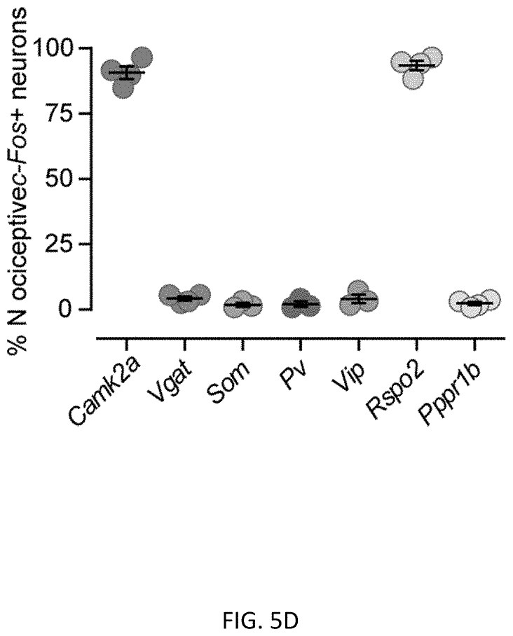

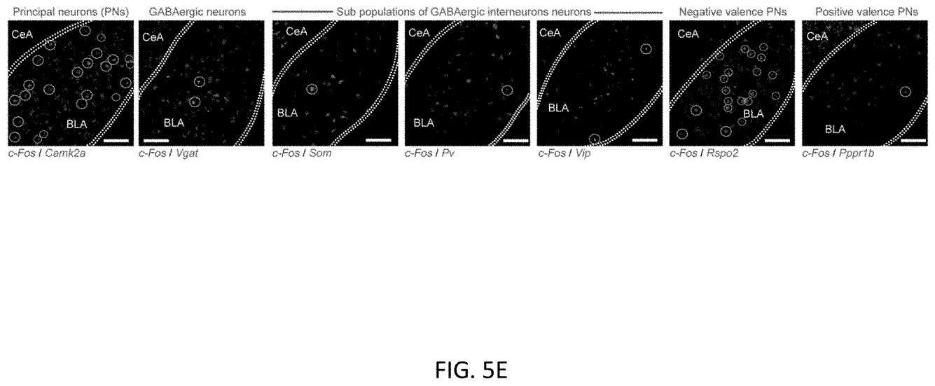

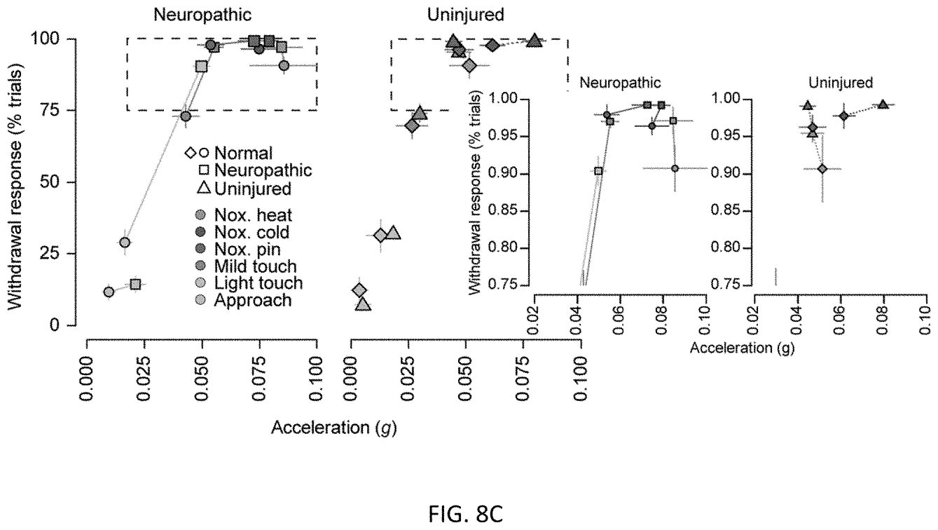

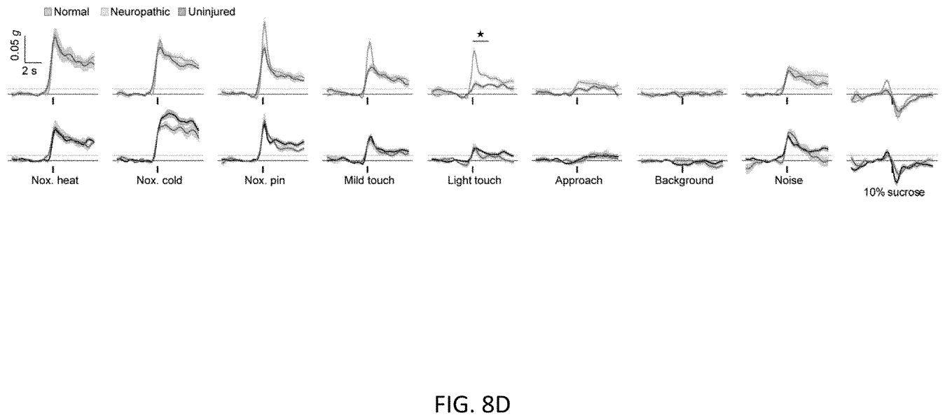

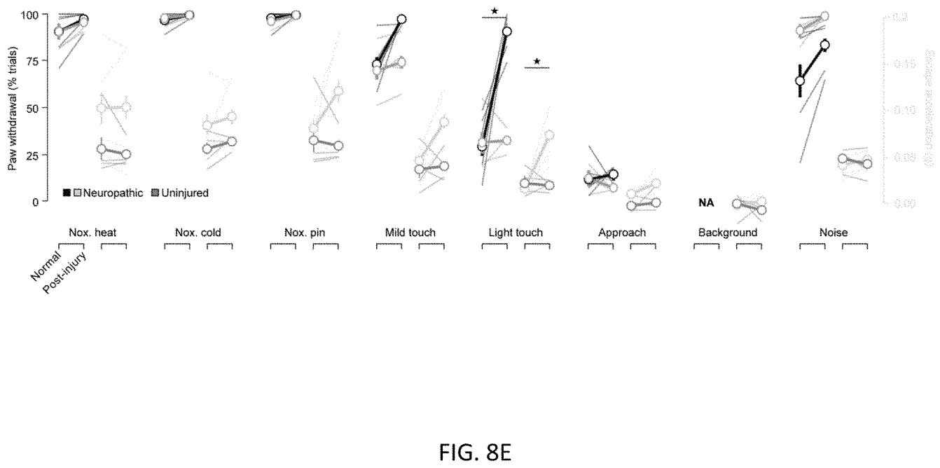

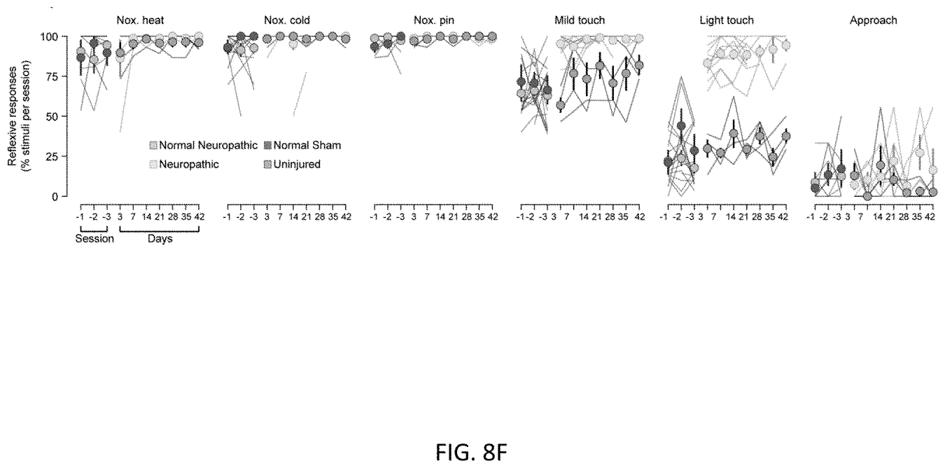

Previous studies attempting to define pain affect mechanisms recorded the acute nociceptive responses of single amygdalar neurons in anesthetized animals (11, 18). However, recent work has shown that the BLA encodes information via the coordinated dynamics of neurons within large ensembles (19); it is therefore important to resolve how the BLA processes pain affect at the neural ensemble level in awake, freely behaving animals. We first performed fluorescence in situ hybridization studies and used the immediate-early gene marker of neural activity, c-Fos, to determine that c-Fos+ neurons activated by nociceptive stimuli comprised a population of mid-anterior BLA Camk2a⁺ principal neurons (FIG. 5). To identify how the BLA encodes nociceptive information, we used a head-mounted miniature microscope to track the somatic Ca²⁺ dynamics of individual BLA Camk2a⁺ principal neurons in freely behaving mice presented with diverse noxious and innocuous stimuli (FIGS. 1A to 1D, and FIGS. 6 and 7) (20). We monitored pain-related behaviors by measuring each animal's locomotor acceleration, which allowed us to track both reflexive withdrawal and affective-motivational behaviors that include attendance to the stimulated tissue and escape (FIGS. 1A and 1E, and FIG. 8).

Noxious heat, cold, and pin prick stimuli elicited significant Ca²⁺ responses in 15±2% (SEM), 13±2%, and 13±2% of active BLA neurons, respectively [3397 neurons (117±8 neurons per session)] (FIGS. 1F to 1H, and table 1). Innocuous light touch induced Ca²⁺ activity in a smaller subset of neurons (7±1%) (FIGS. 1F and 11, and FIG. 9E). Alignment of all stimulus-evoked ensemble responses to the noxious heat trials revealed an overlapping population of principal neurons that encoded nociceptive information across pain modalities (i.e., noxious heat, cold, pin), which we refer to here as the BLA nociceptive ensemble (24±2% of active BLA neurons) (FIGS. 1F to 1I).

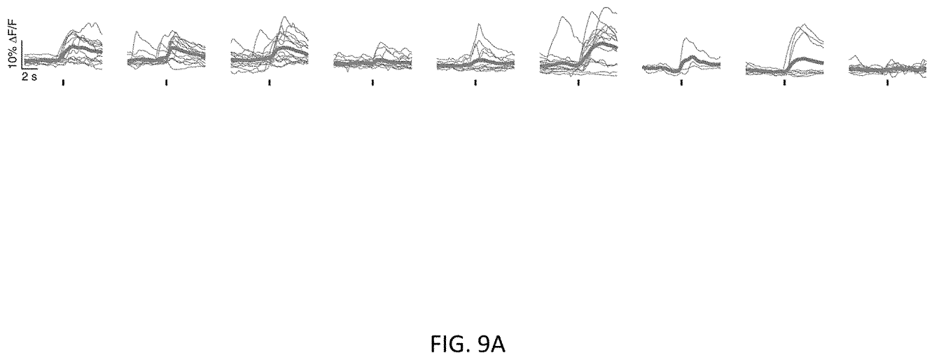

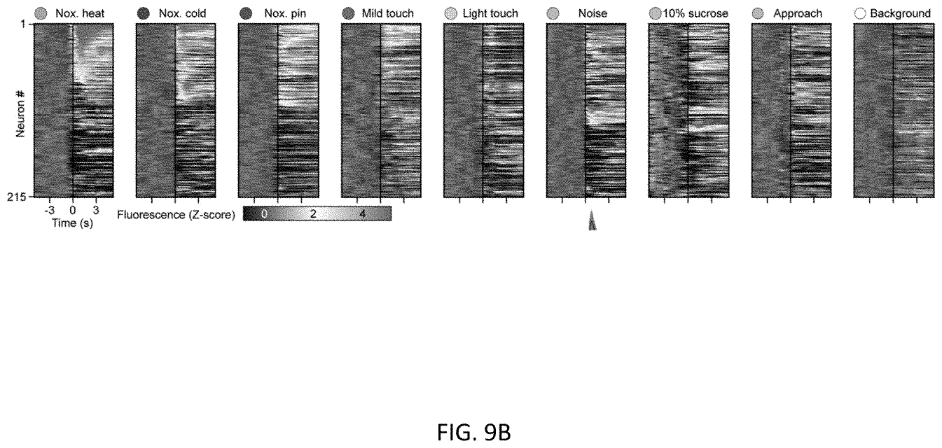

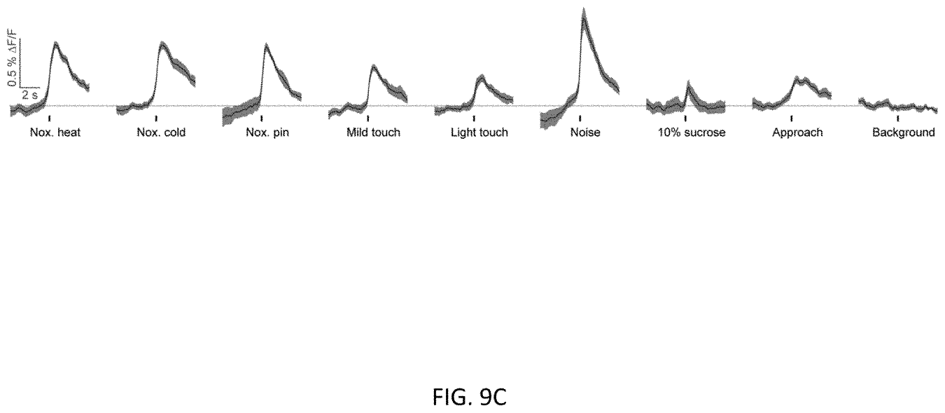

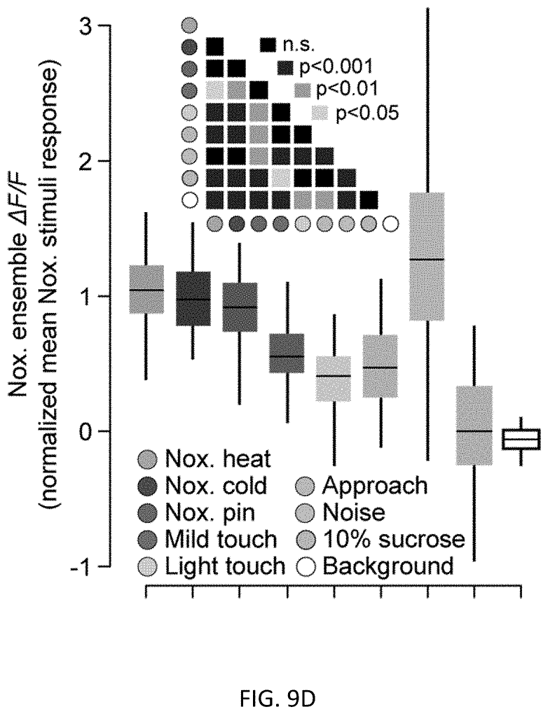

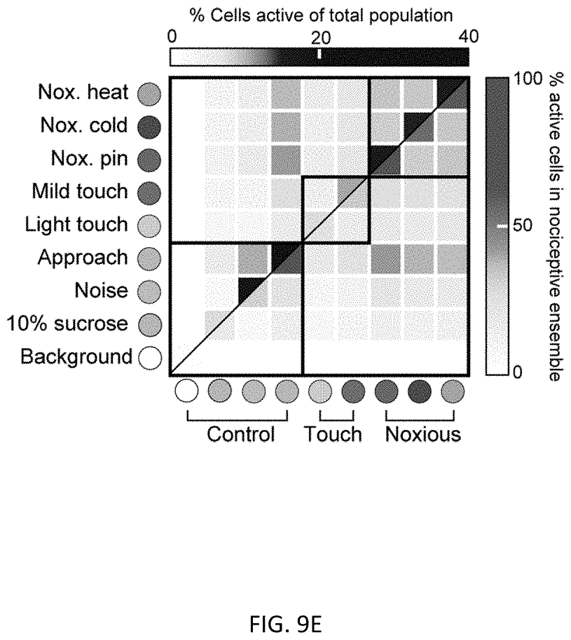

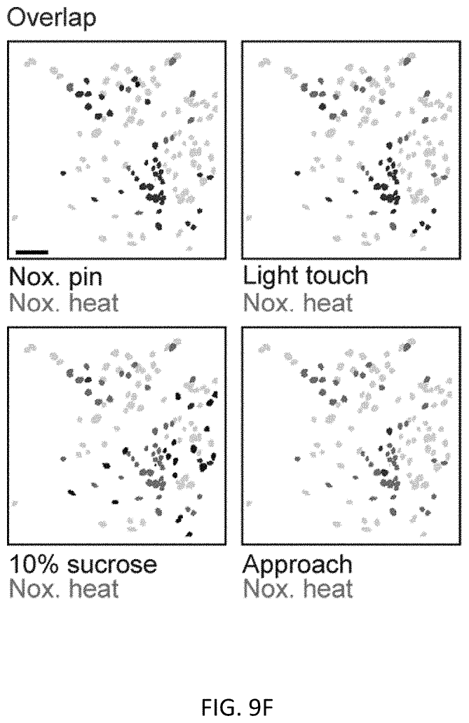

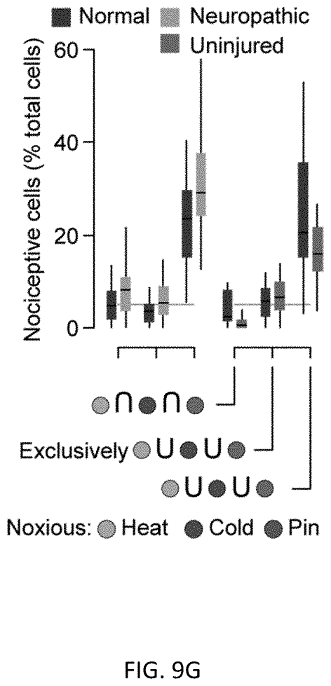

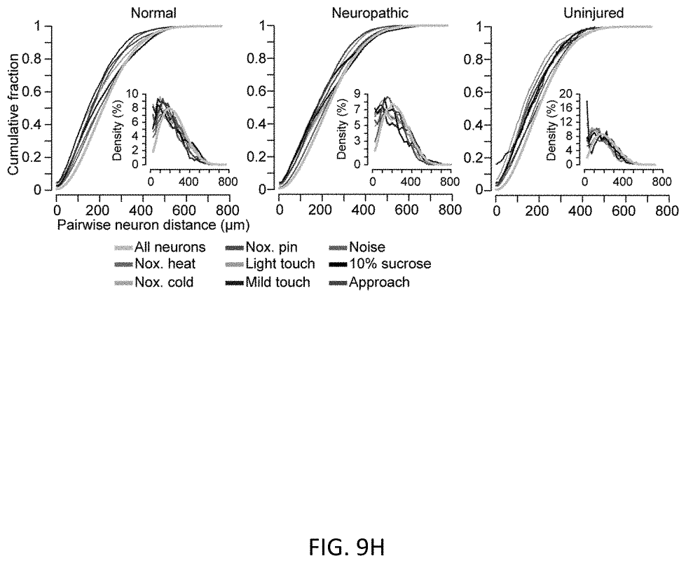

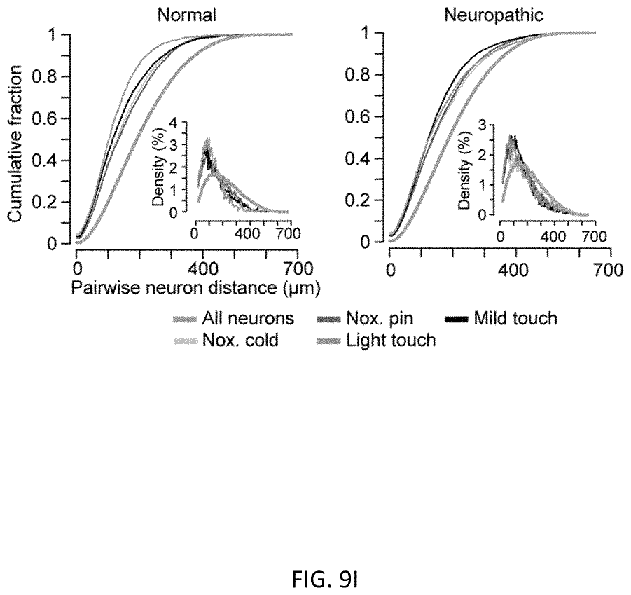

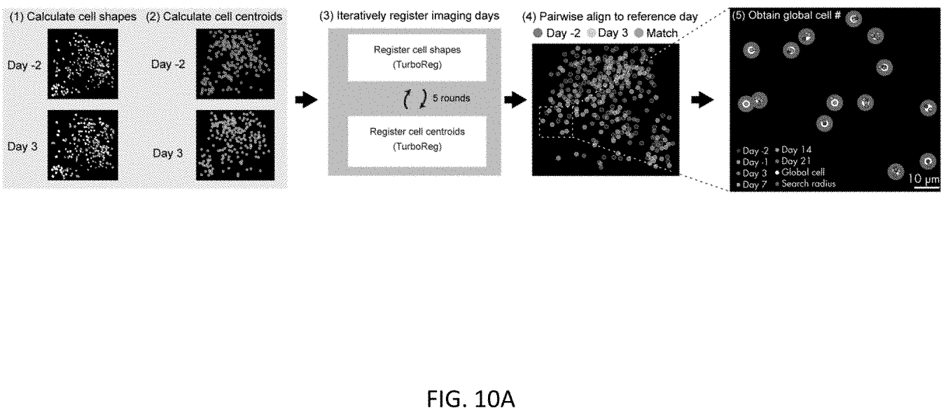

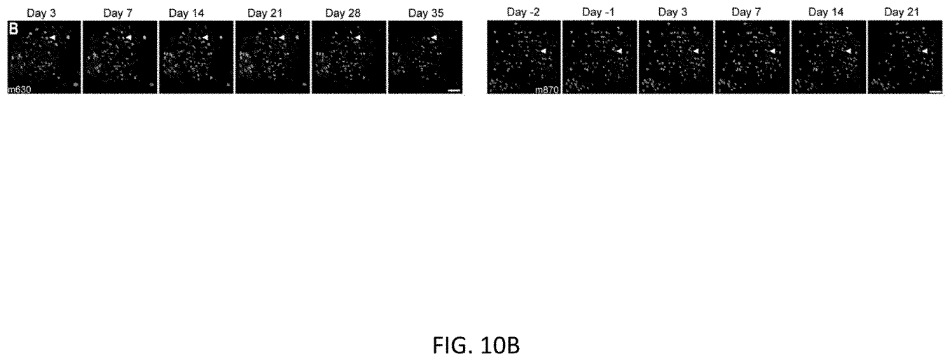

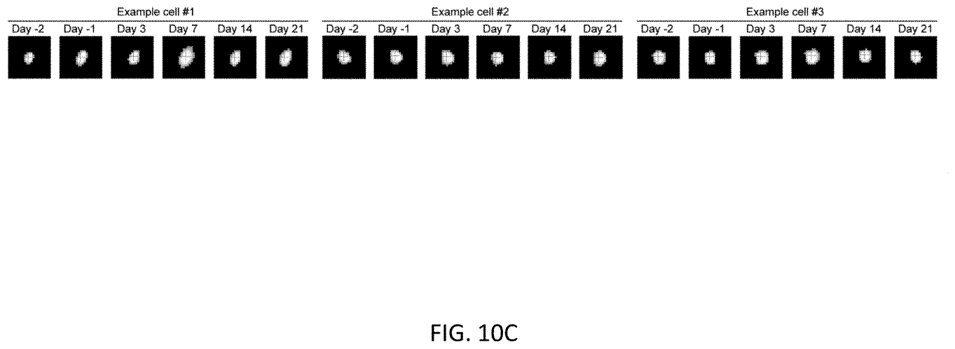

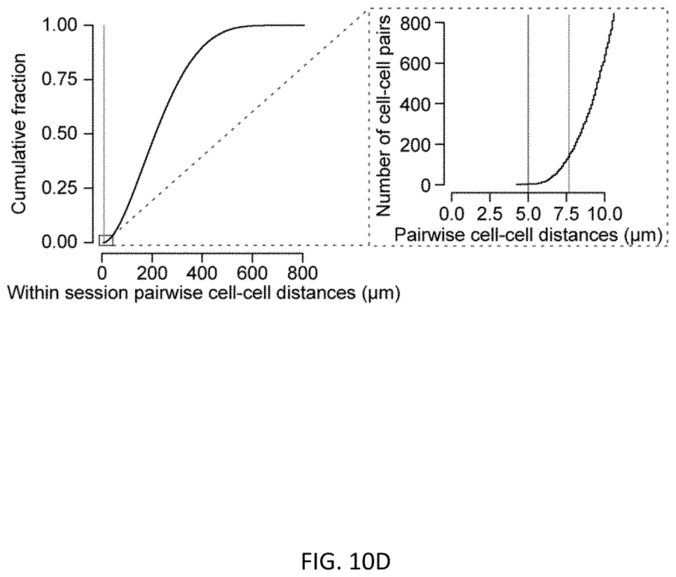

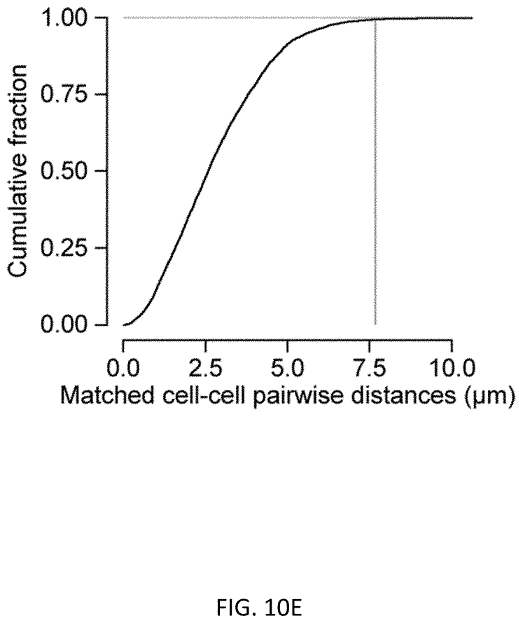

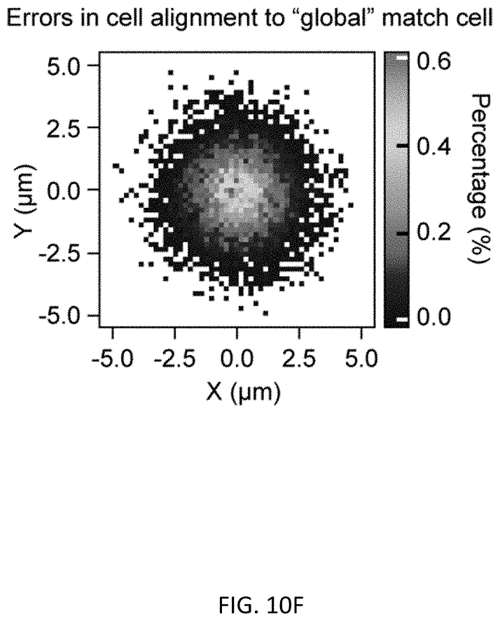

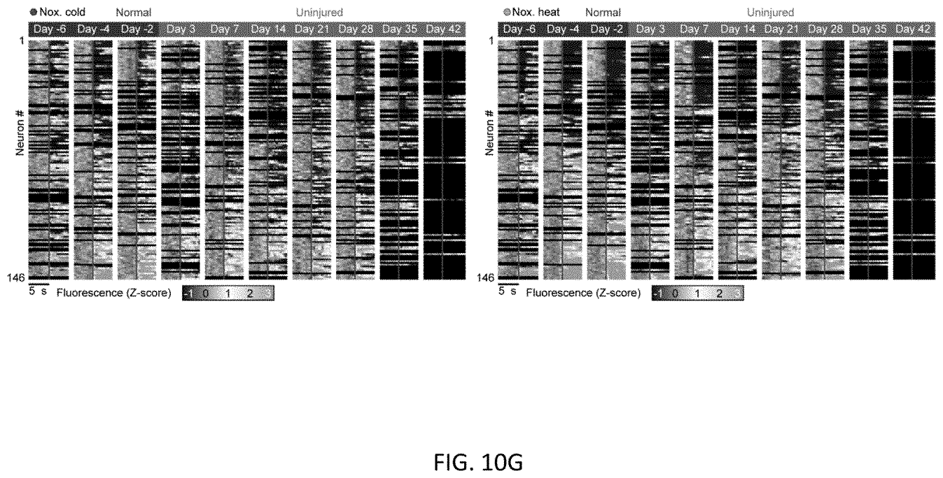

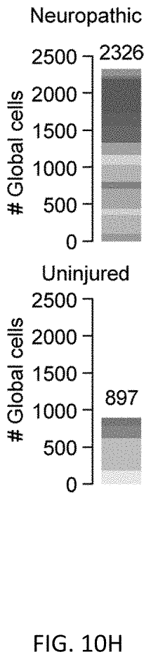

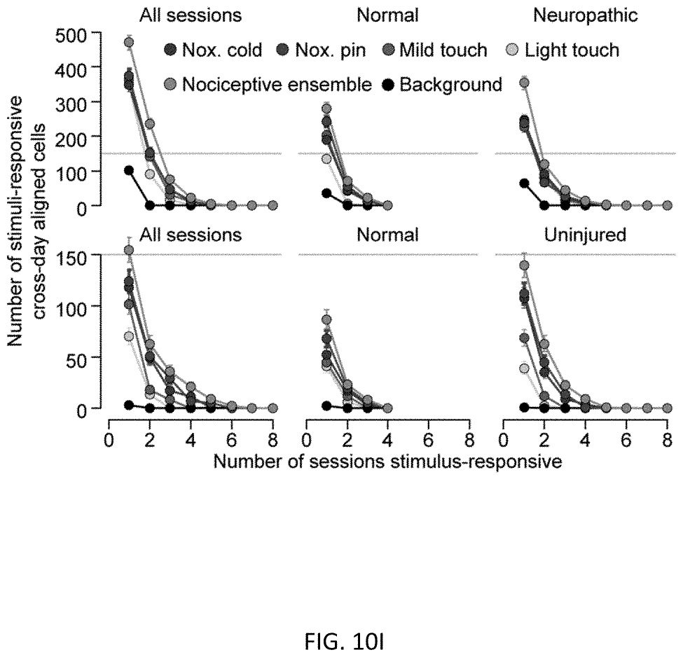

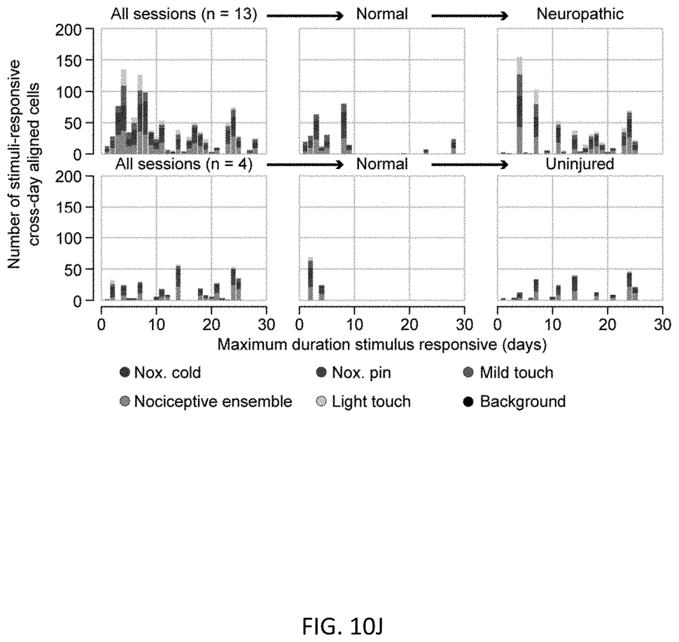

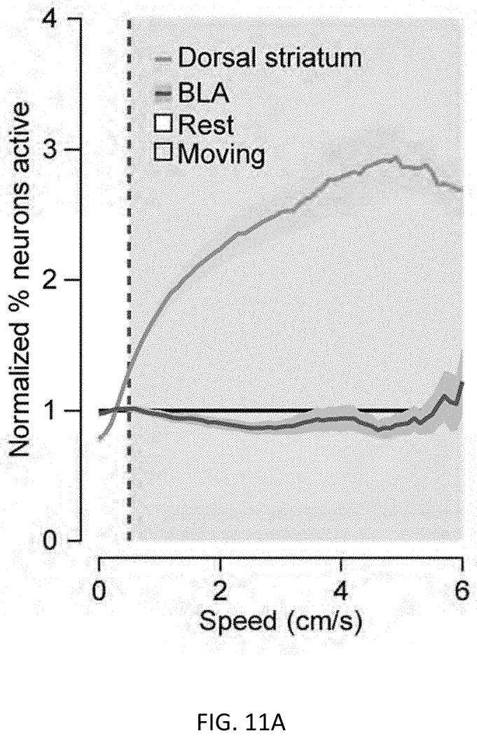

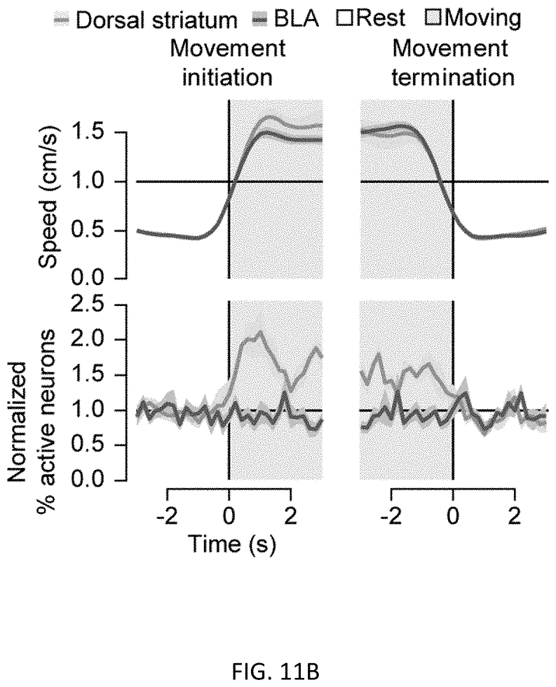

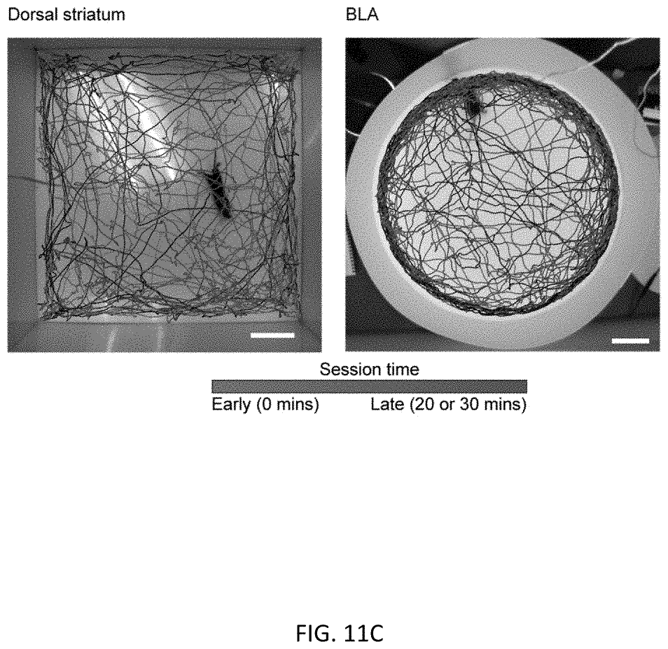

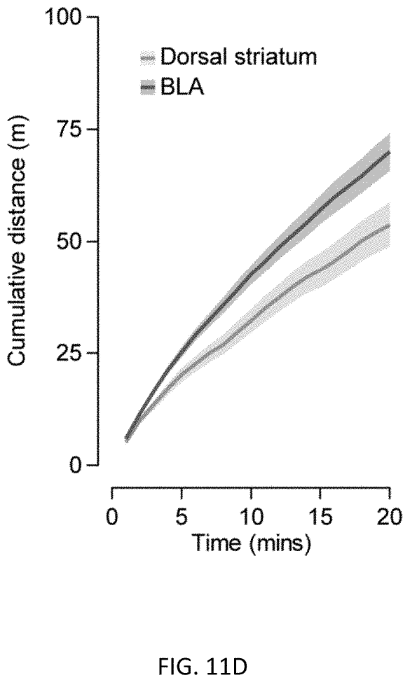

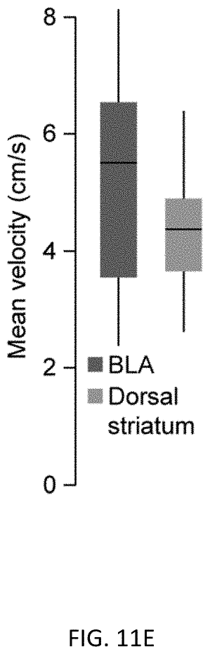

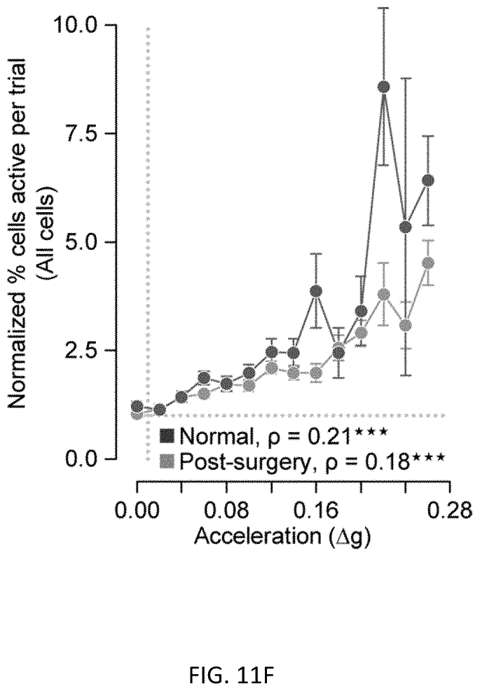

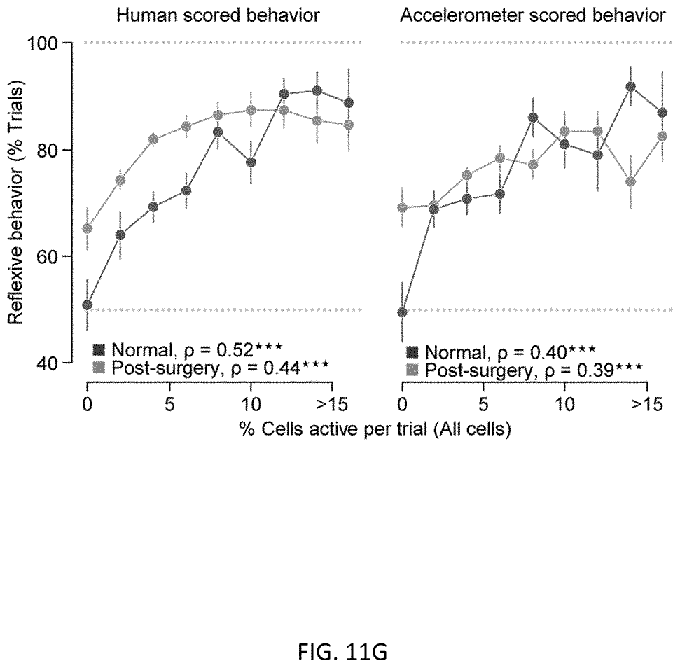

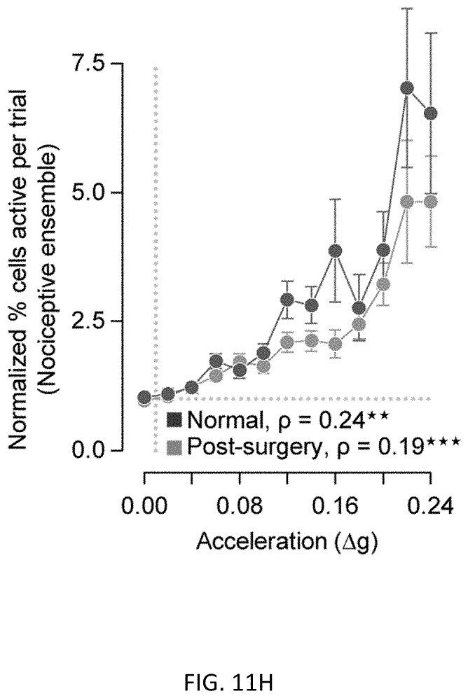

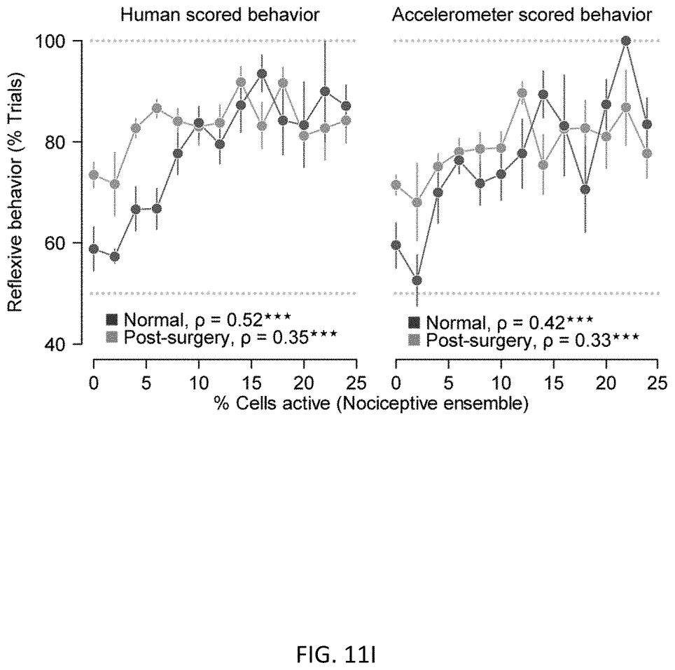

This ensemble was composed of multimodal responsive neurons, as well as a unique population that appeared to encode nociception selectively and no other sensory information (6±1% of all imaged neurons) (FIG. 1K and FIG. 9G). Pain behavioral responses evoked by noxious stimuli closely mirrored the activity of this nociceptive neural ensemble (FIGS. 1E and 1G, and FIGS. 8D and 8E). The nociceptive ensemble contained a subset of neurons that maintained their noxious stimulus response properties for more than a week (11% of 3223 cross-day-aligned neurons) (FIG. 10). Increasingly salient stimuli, from light touch (18±3% of the nociceptive ensemble) to mild touch (31±4%), activated larger subsets of the nociceptive ensemble (FIGS. 1G and 11, FIGS. 9D and 9E, and Table 1) and induced heightened behavior (FIG. 1E and FIG. 8). Expectation of stimulus contact (“approach/no contact” trials) also evoked sparse BLA activity (7±2% of the total population) (FIGS. 9A to 9E, and Table 1). BLA activity did not correlate with exploratory locomotion (FIGS. 11A to 11E) (21).

To determine whether the BLA nociceptive ensemble broadly encodes stimulus valence (22, 23), we presented mice with an appetitive stimulus (10% sucrose). Sucrose consumption was encoded by a distinct ensemble (18±3% of all neurons) that only overlapped with a subset of neurons in the nociceptive ensemble (7% of total neurons) (FIG. 1J and FIG. 9E) (19). Similar to conditioned responsive valence networks (23), neurons encoding unconditioned nociceptive and appetitive information were spatially intermingled (FIGS. 9F, 9H, and 9I). Consistent with these results, nociceptive c-Fos+ neurons expressed the negative valence marker gene Rspo2 but not the positive valence marker gene Pppr1b (24) (FIGS. 5D and 5E).

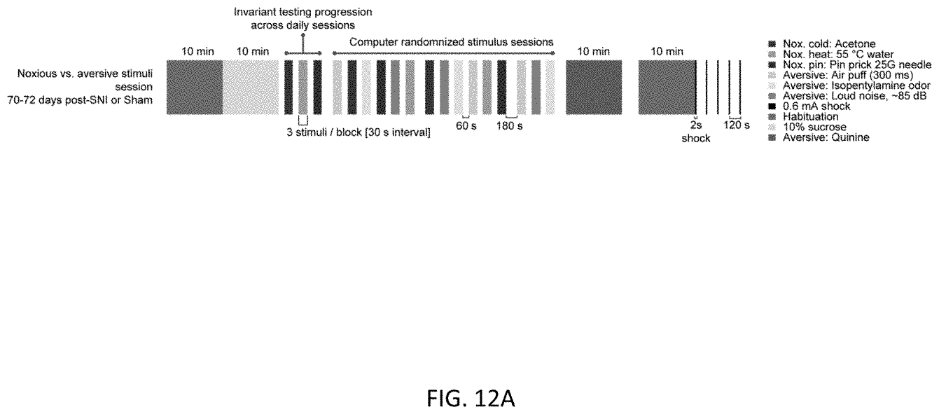

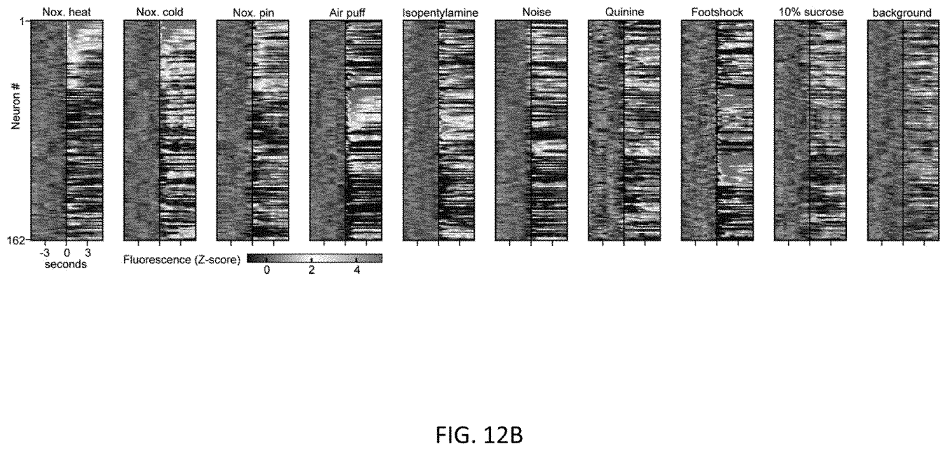

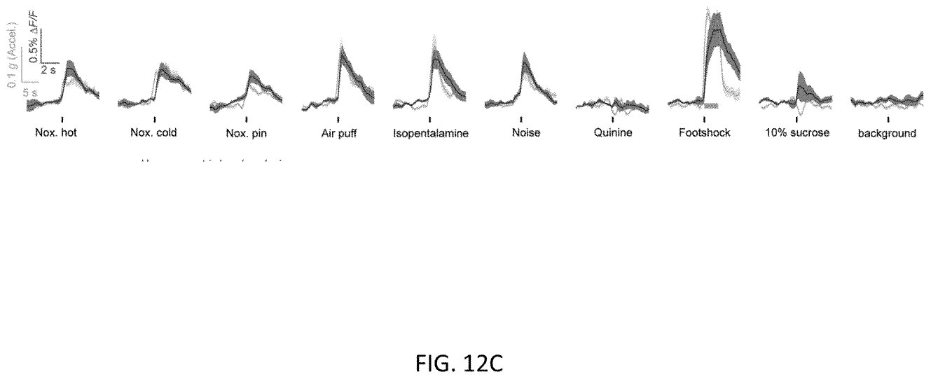

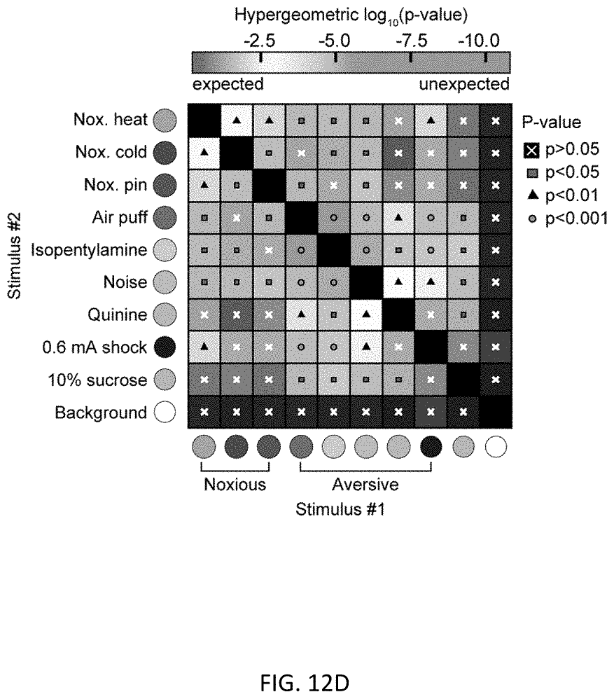

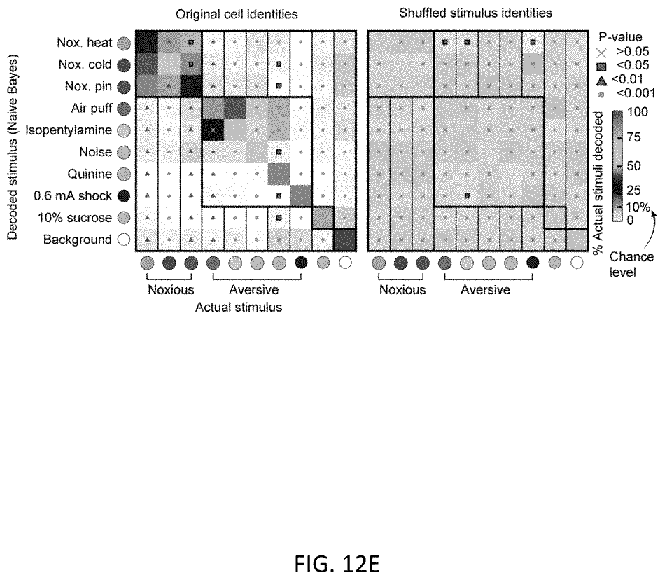

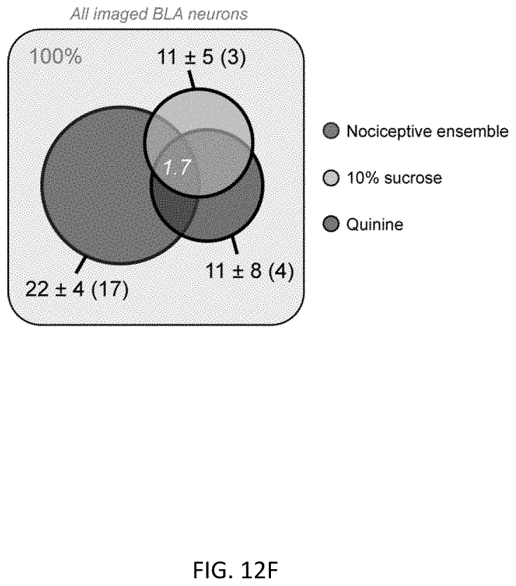

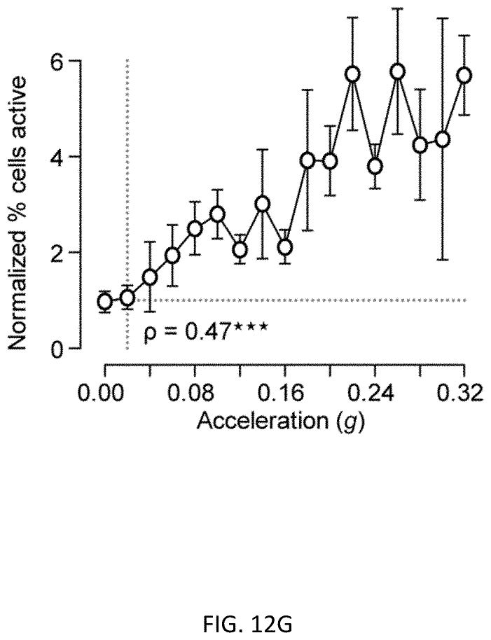

We next determined if the nociceptive ensemble was engaged during aversive experiences other than pain by presenting a panel of sensory, but nonsomatosensory or nonnaturalistic, aversive stimuli, including repulsive odor, bitter taste, loud tone, facial air puff, and electric shock. We found that while there was overlap between the neural ensembles that encode nociceptive, aversive, and electric shock stimuli (˜10% of all imaged neurons), there remained a subset of BLA neurons (˜6% of imaged neurons) that responded only to naturalistic nociceptive stimuli (FIG. 1K and FIG. 12).

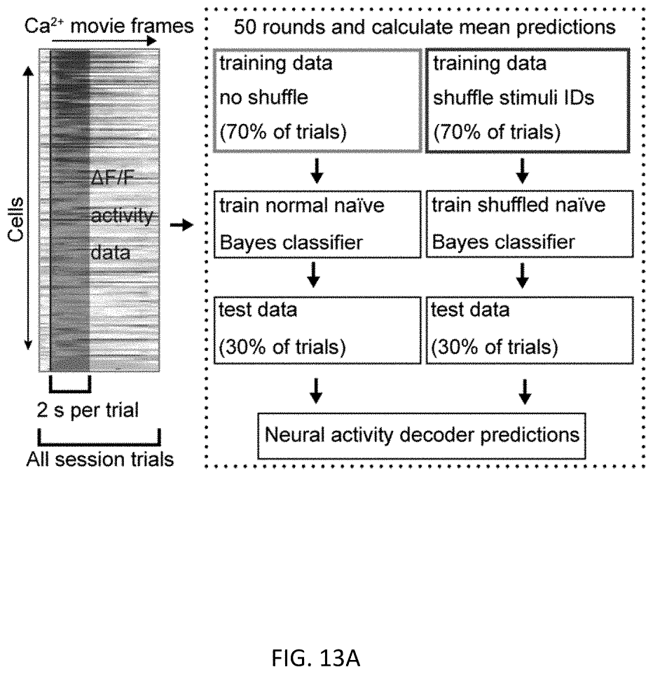

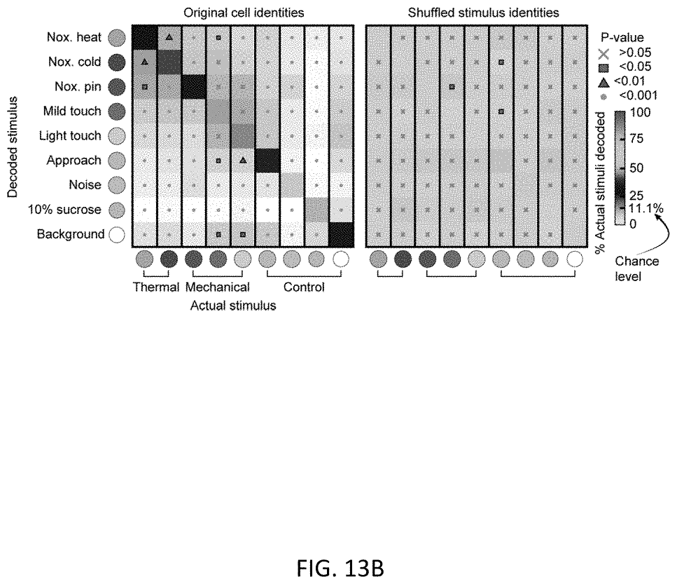

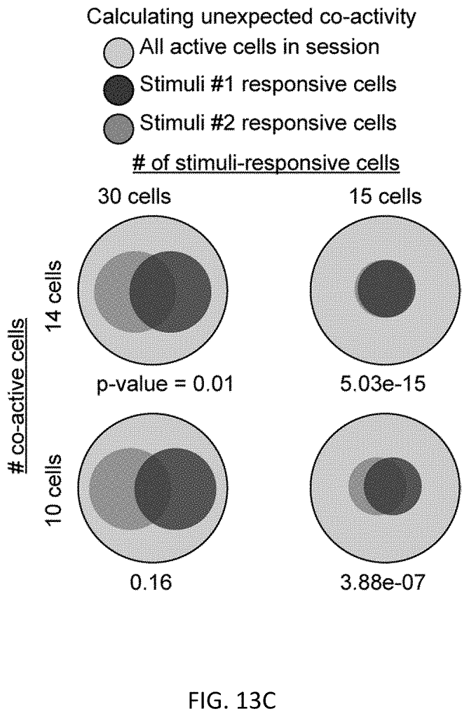

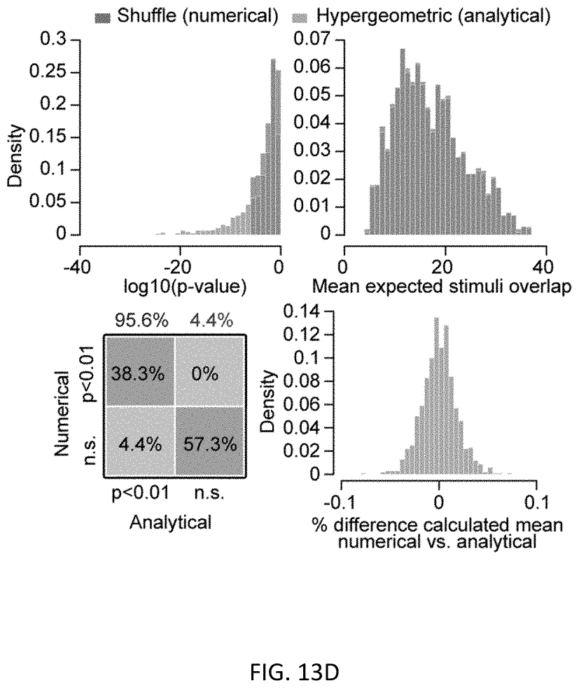

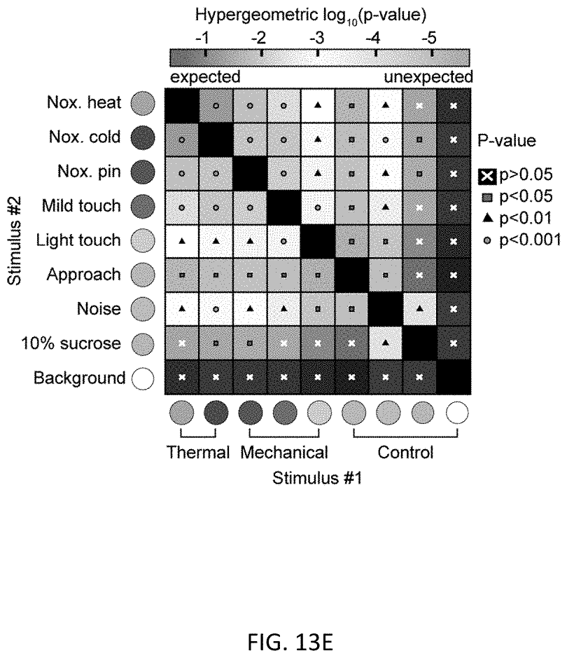

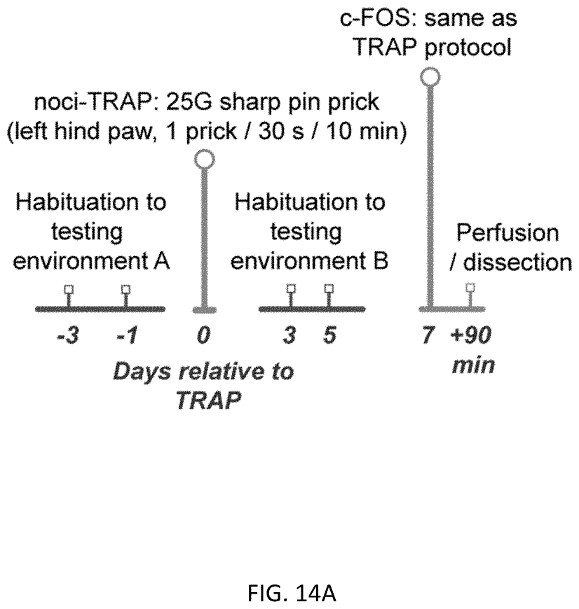

By analyzing the neural ensemble dynamics with pattern classification methods, we were able to classify and distinguish with high accuracy noxious stimuli from other aversive stimuli (FIG. 12E), supporting the finding that noxious stimuli induce a distinct mode of BLA activation. Moreover, sensory stimuli of different valences, intensities, and modalities are represented by unique activity codes. Noxious stimuli were encoded distinctly from one another and could be distinguished with even higher fidelity from innocuous, non-nociceptive aversive, and appetitive stimuli (FIG. 1L and FIGS. 13A and 13B), indicating that there is a core set of BLA neurons that encodes nociceptive stimuli via specific dynamic neural codes. One crucial finding was that greater activation of this BLA nociceptive ensemble was predictive of increased pain behaviors, suggesting that BLA nociceptive processing influences the magnitude of pain behaviors (FIG. 1M and FIGS. 11H and 11I).

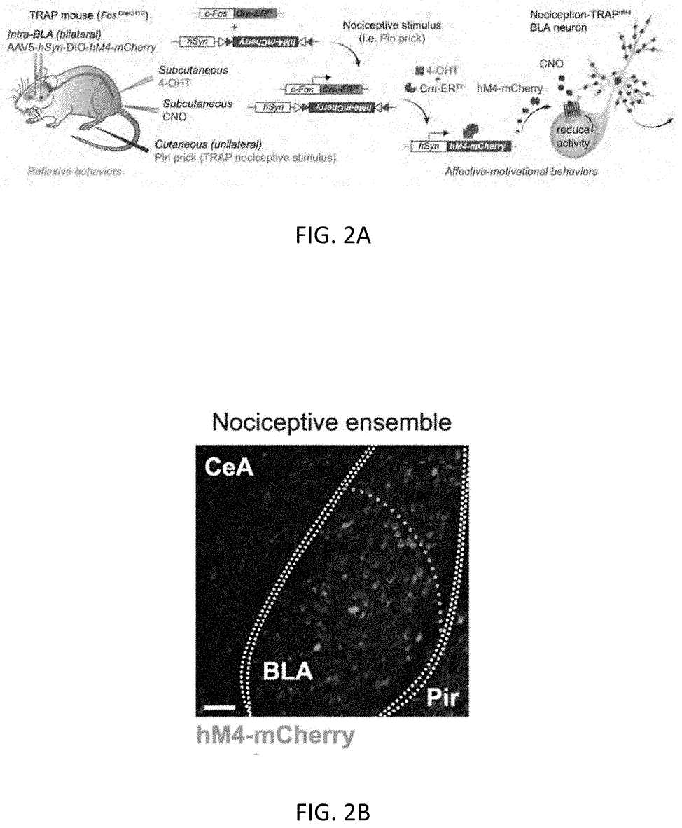

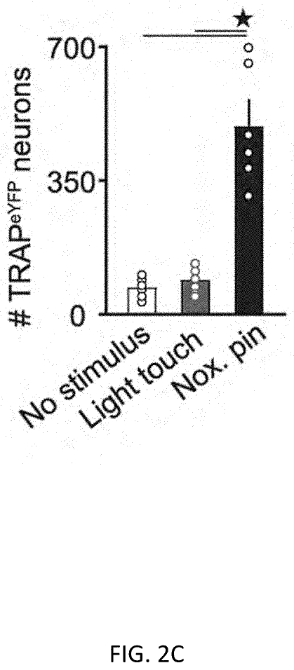

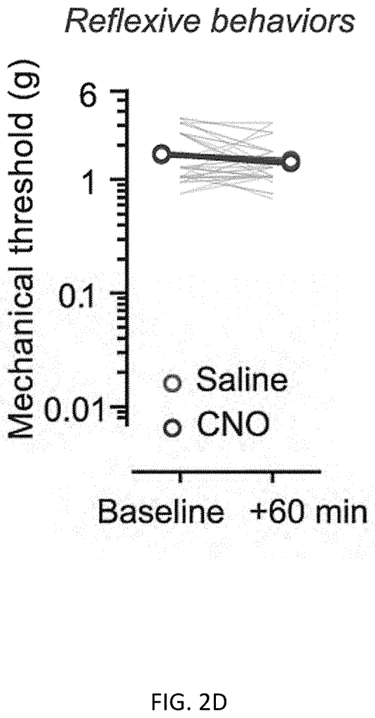

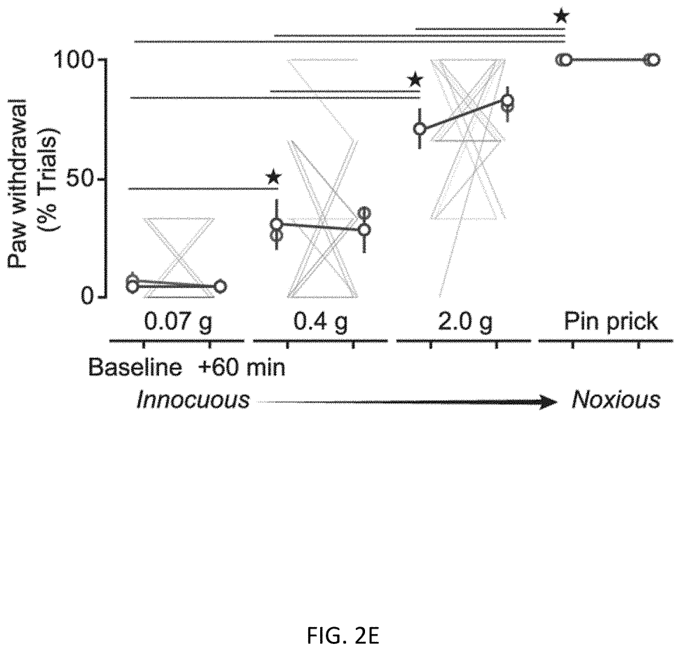

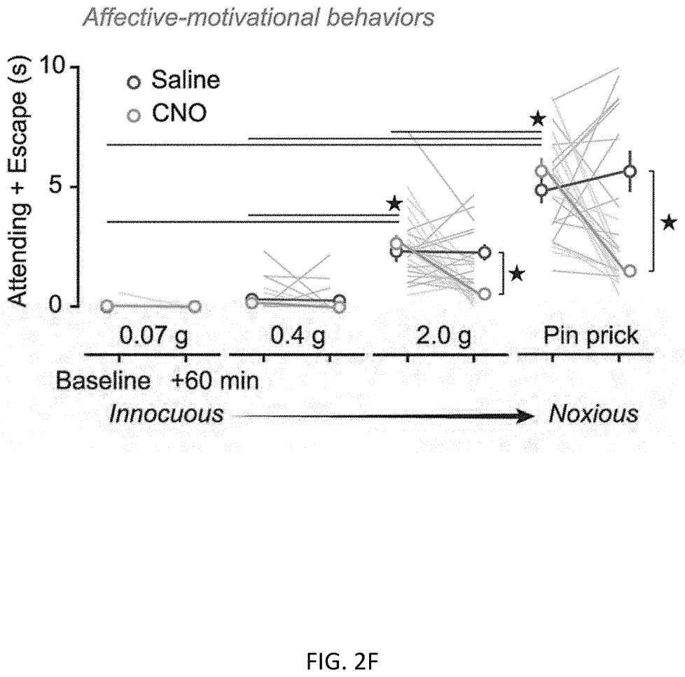

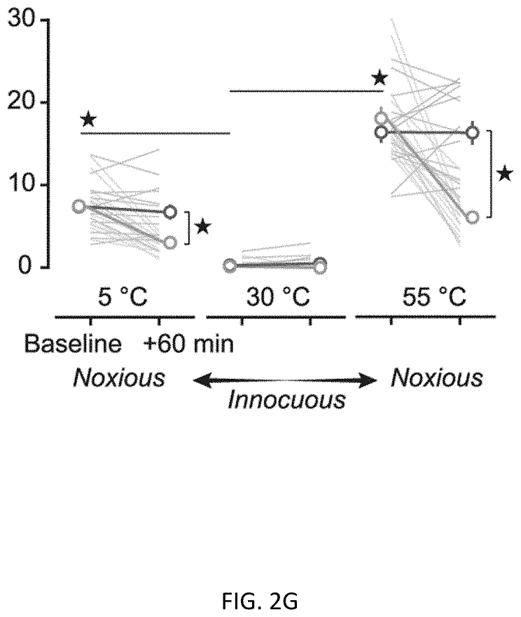

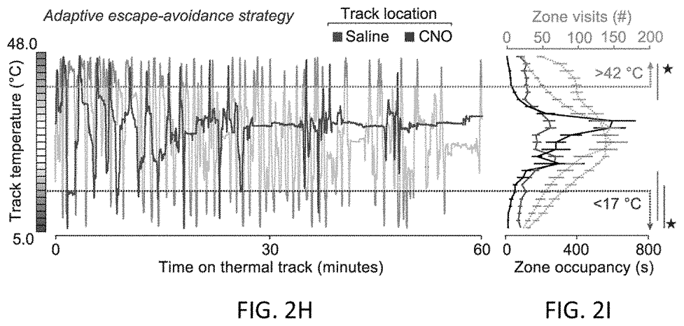

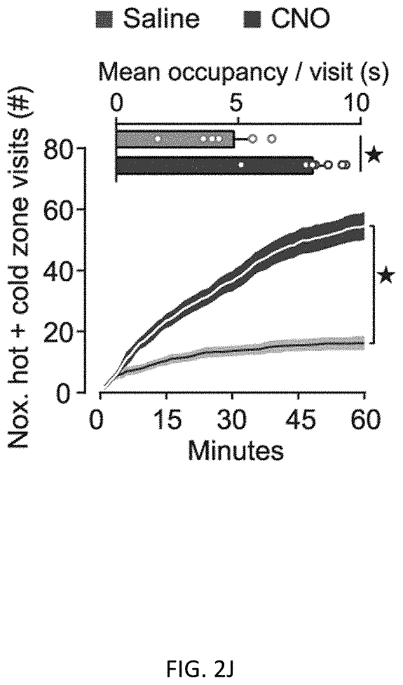

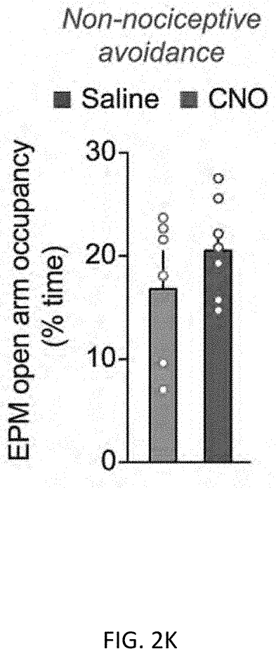

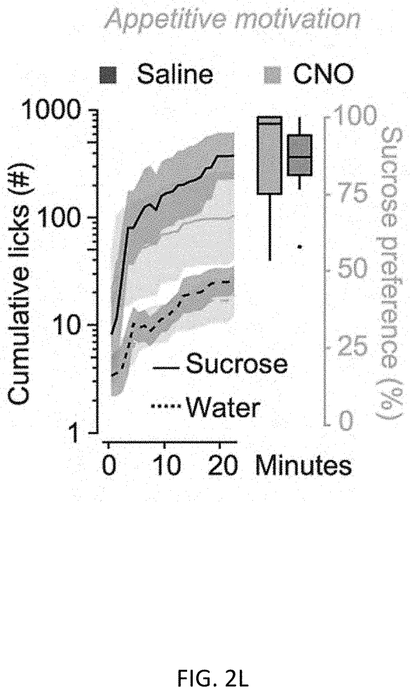

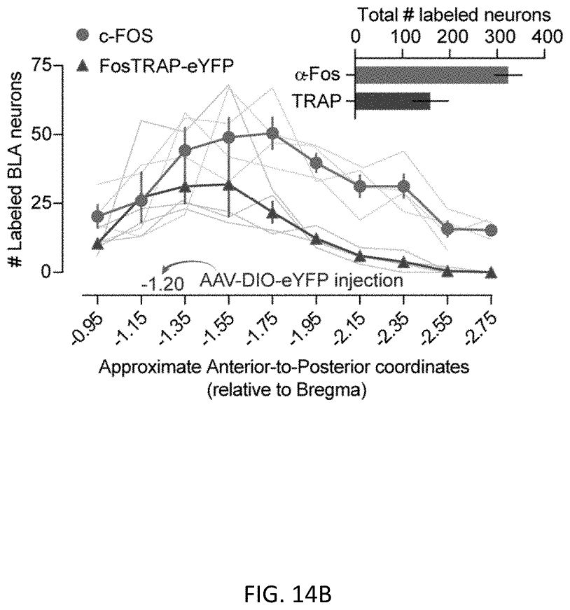

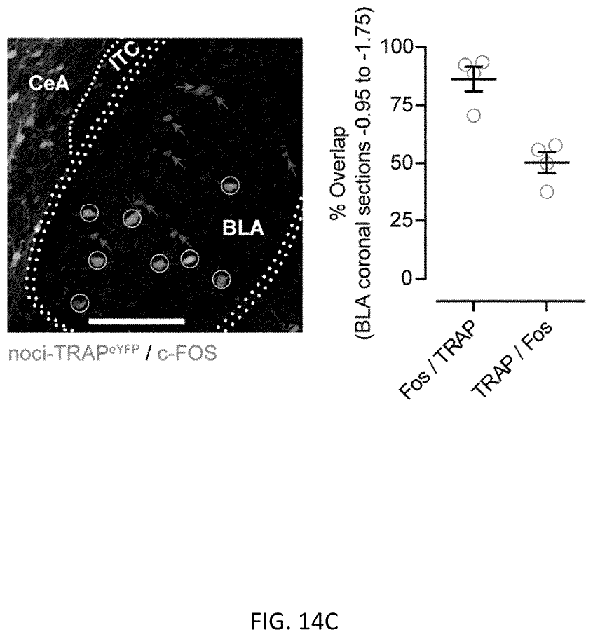

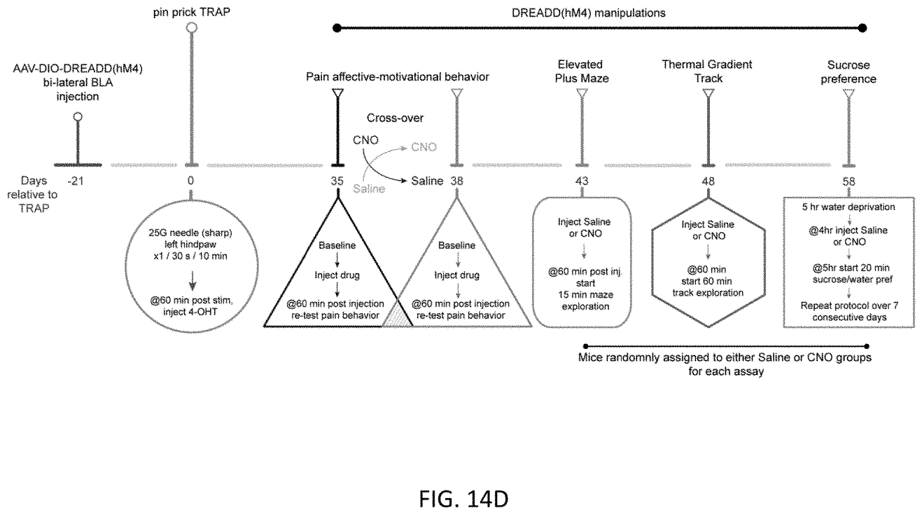

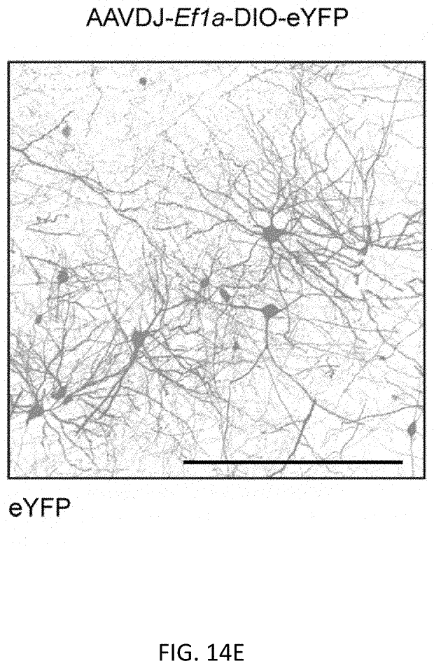

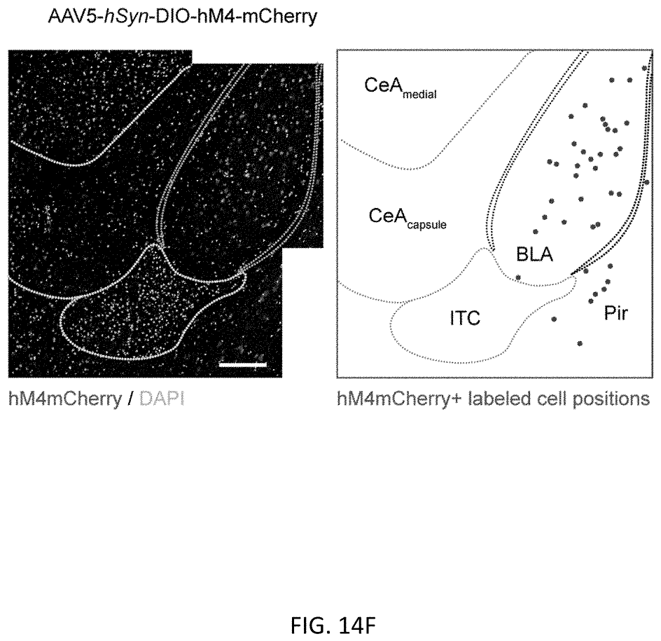

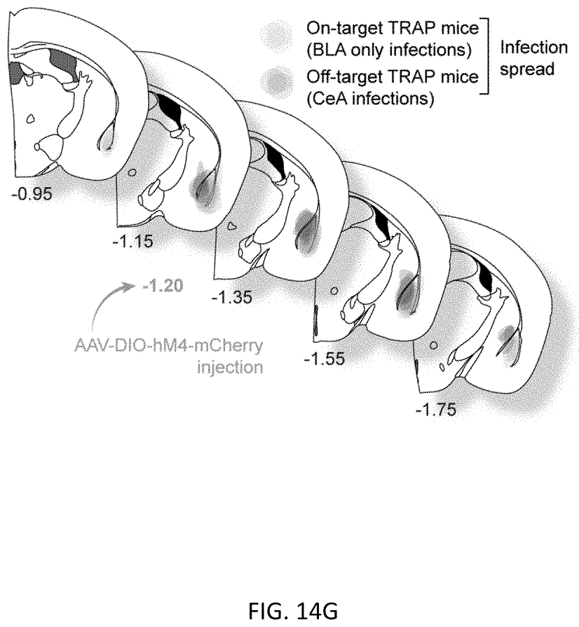

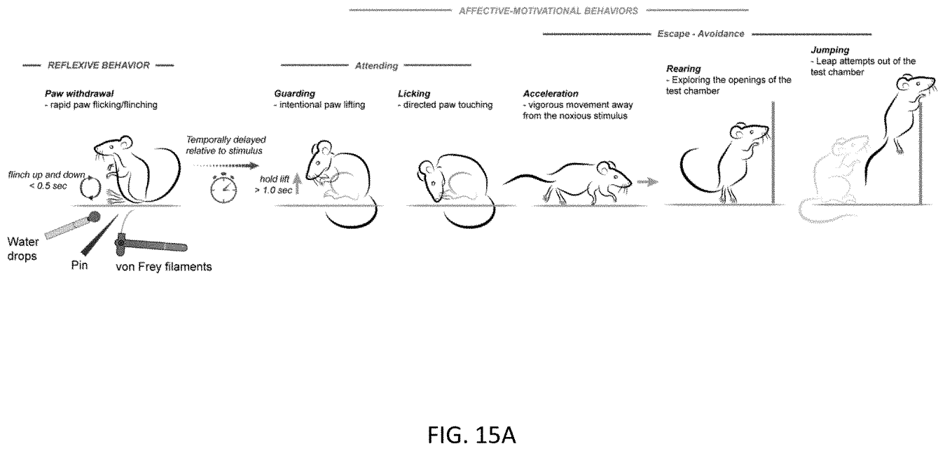

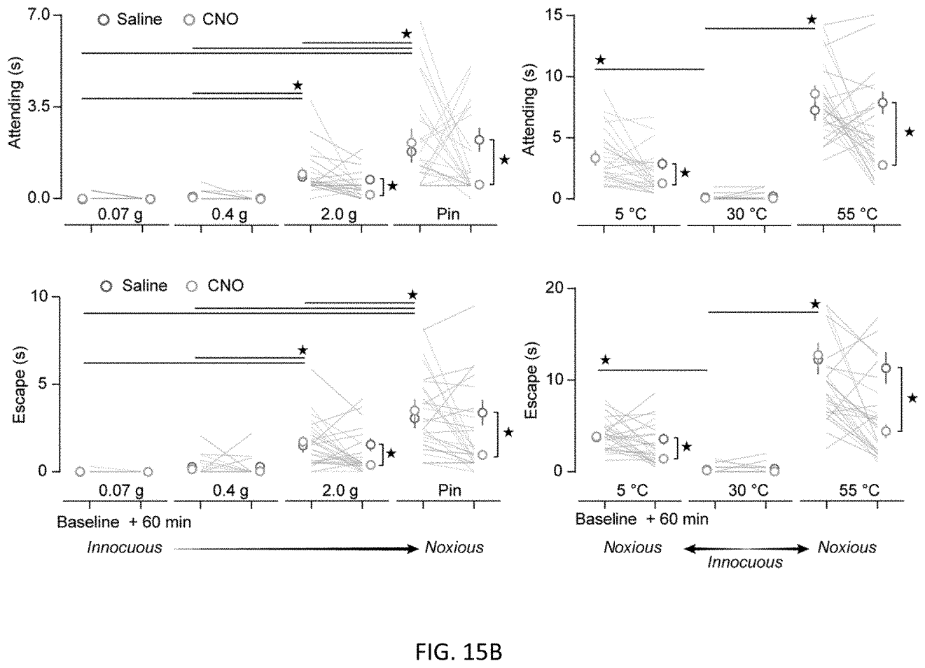

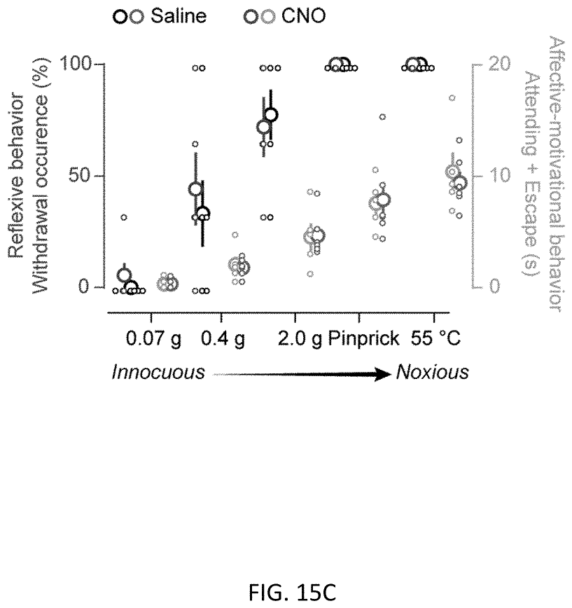

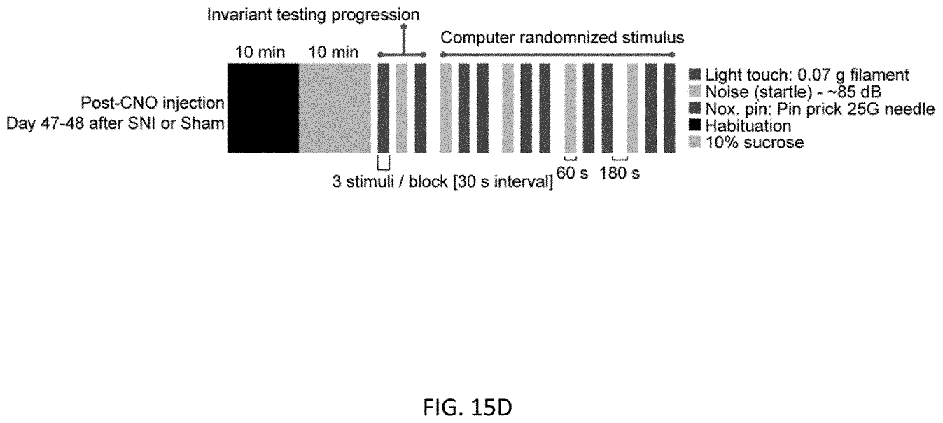

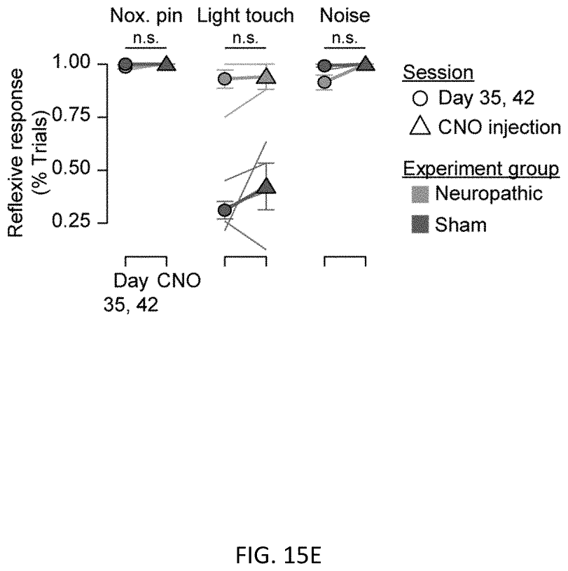

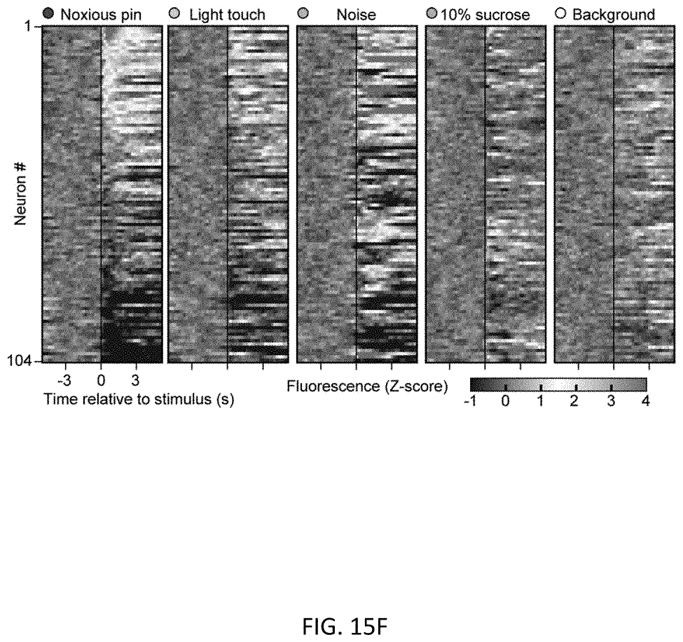

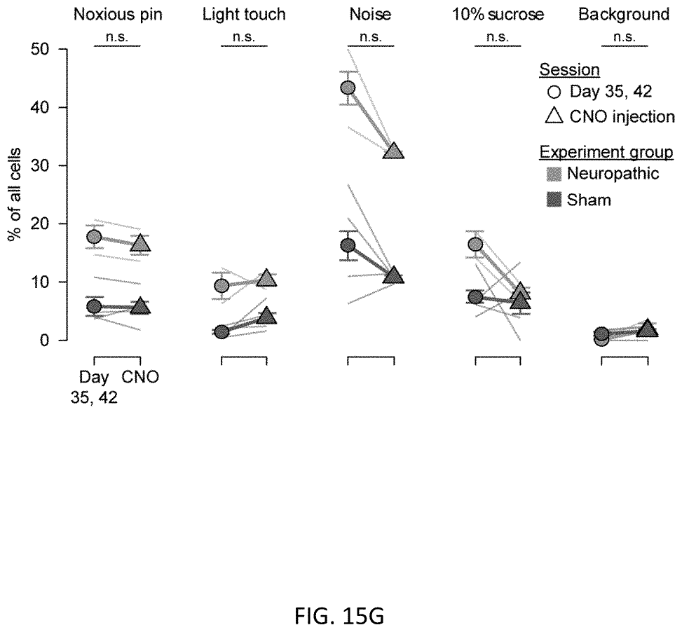

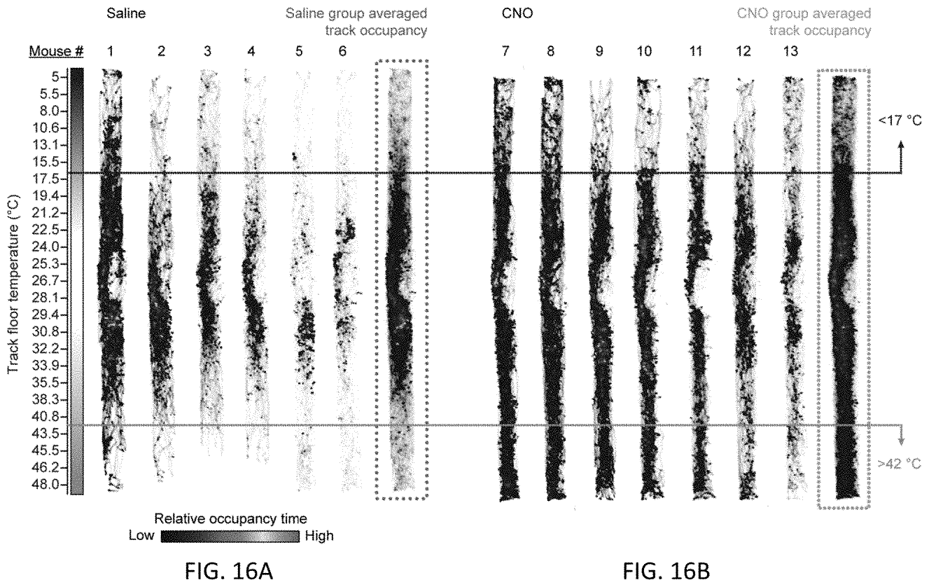

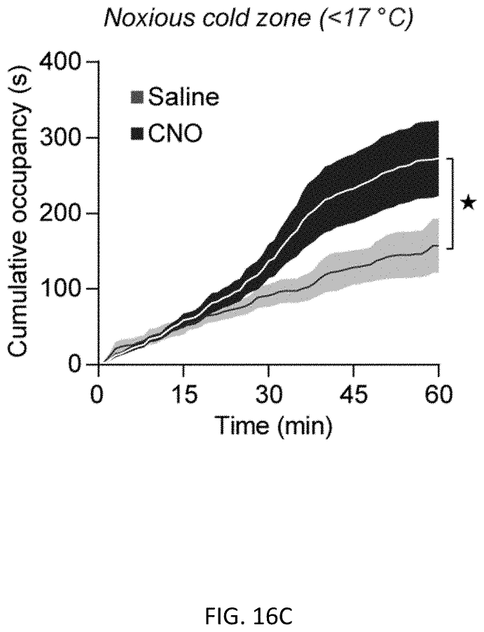

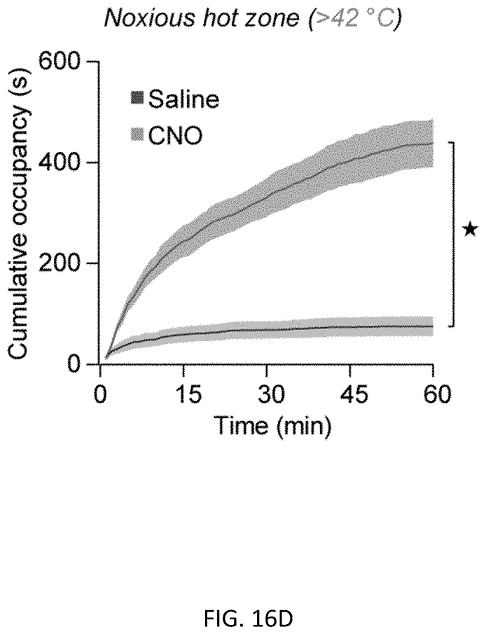

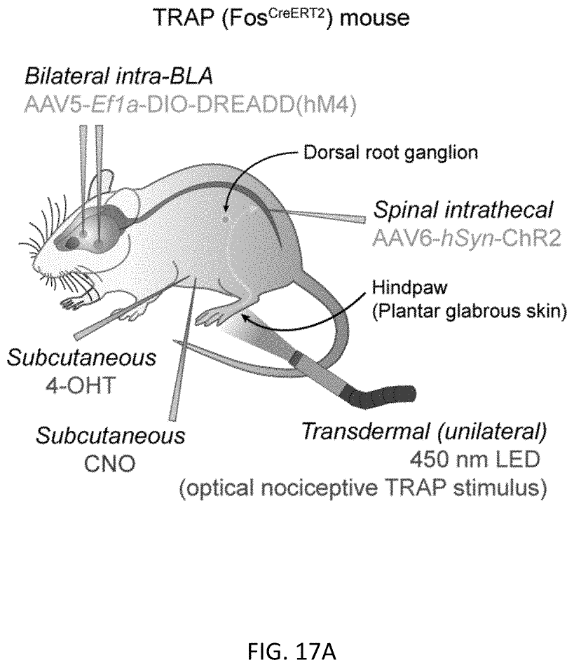

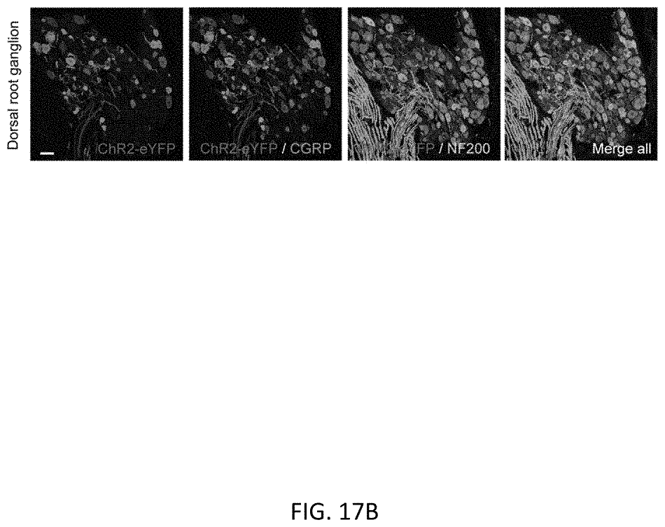

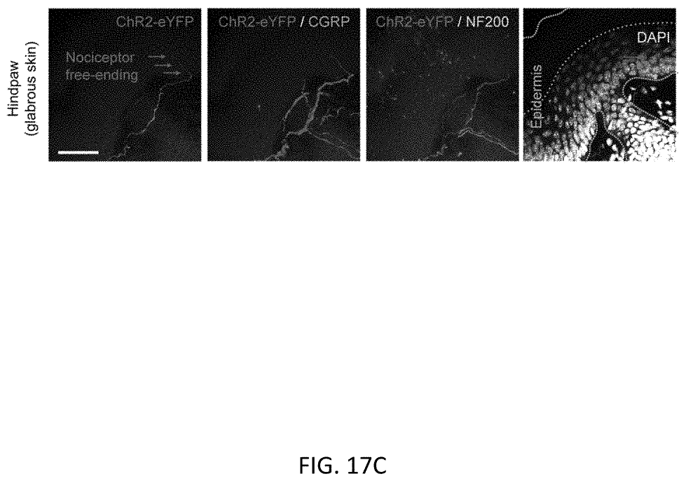

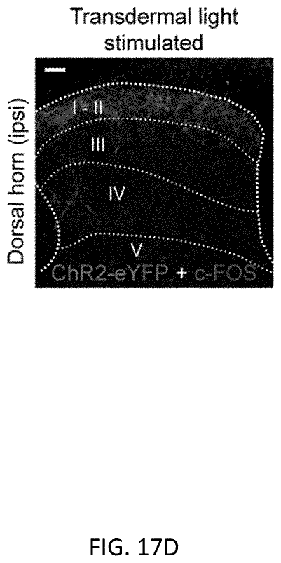

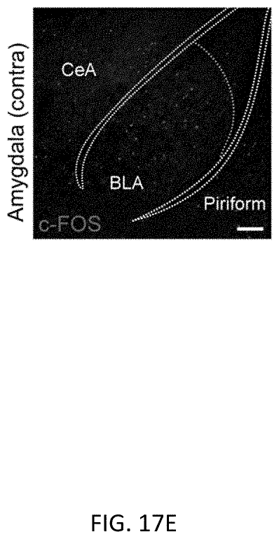

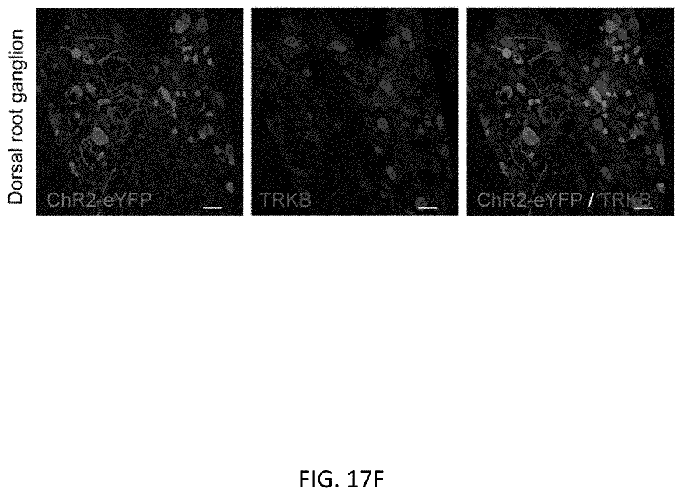

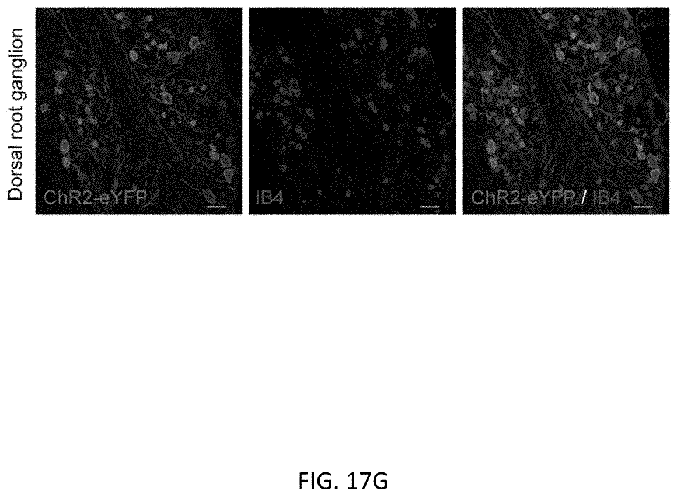

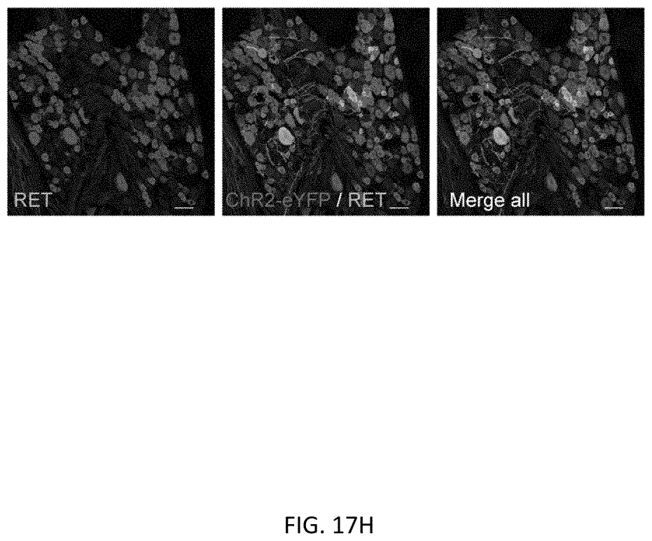

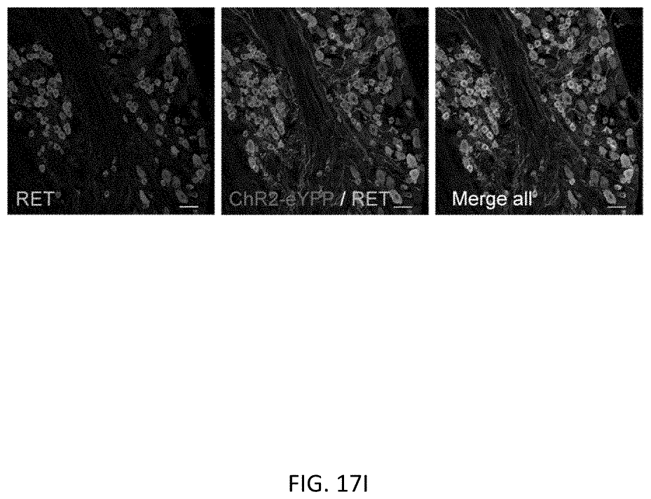

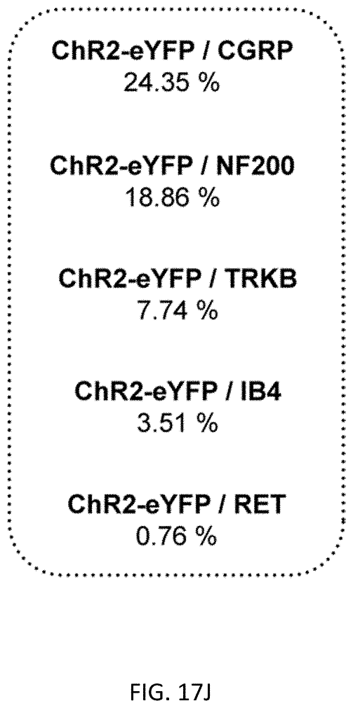

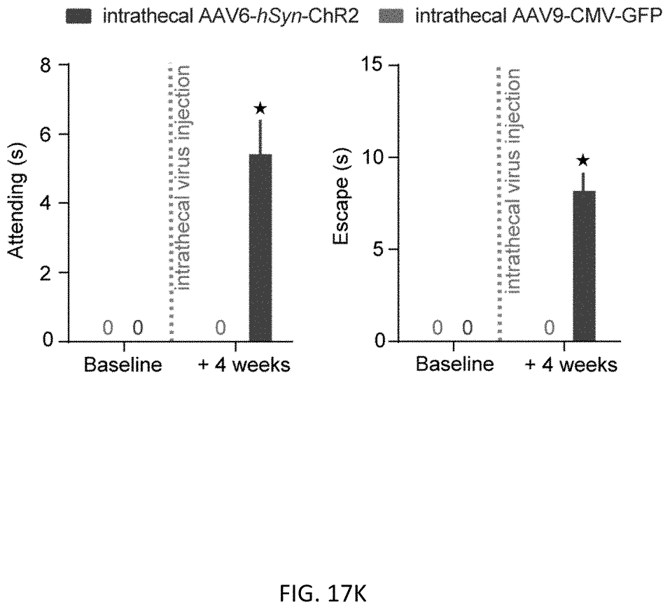

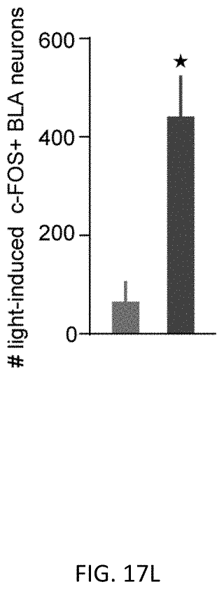

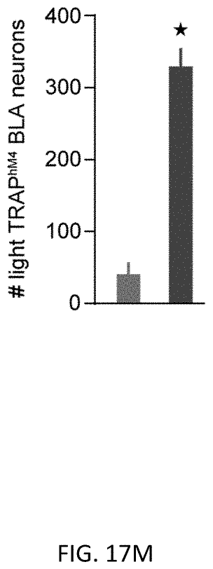

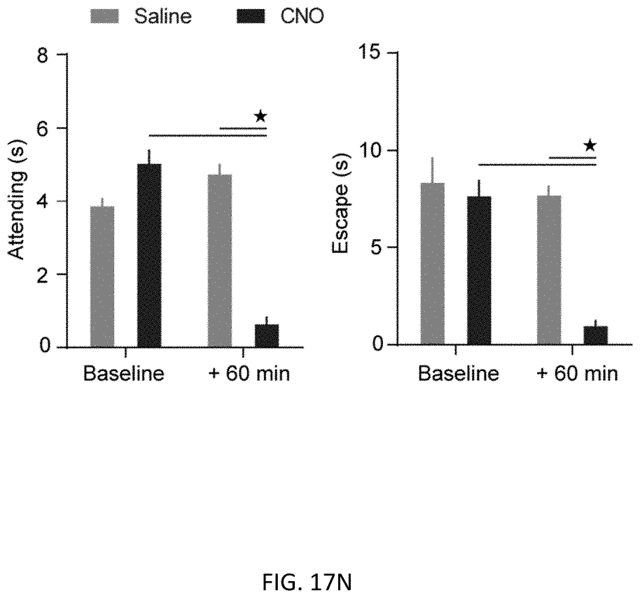

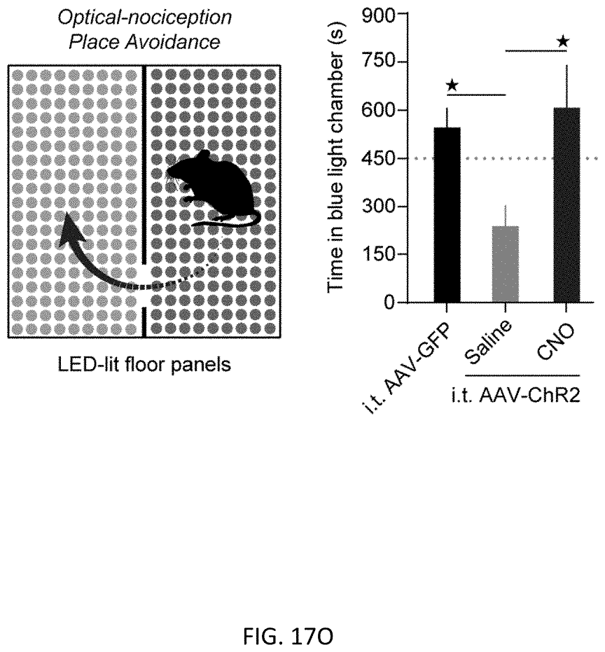

To test the causal role of the BLA nociceptive ensemble for pain behaviors, we expressed a Cre-dependent inhibitory DREADD neuromodulator (hM4-mCherry) in mutant TRAP mice (Fos^{CreERT2}) by applying noxious pin pricks that induced activity-dependent, spatially, and temporally controlled DNA recombination and hM4-mCherry expression (noci-TRAP^{hM4} mice) (FIGS. 2A to 2C, and FIG. 14) (25, 26). Since the BLA encodes multiple modalities of nociceptive stimuli within a core ensemble (FIG. 1H), we hypothesized that silencing the neurons activated by noxious pin prick would alter behavioral responses to all types of noxious stimuli. Indeed, the hM4 agonist clozapine-N-oxide (CNO; 10 mg/kg) significantly reduced both attending and escape behaviors, but not stimulus detection and withdrawal, for both mechanical and thermal noxious stimuli (FIGS. 2D to 2G, and FIGS. 15A and 15B). CNO alone had no effect on pain behaviors in control mice (FIG. 15C) (27). To test operant pain behavior, we next allowed noci-TRAP^{hM4} mice to explore a thermal gradient track in which the polar ends were set at noxious cold (5 to 17° C.) and hot (42 to 8° C.) temperatures (FIG. 2H). The noci-TRAP^{hM4} mice injected with control saline rapidly acquired an adaptive avoidance strategy of the noxious zones. In contrast, noci-TRAP^{hM4} mice treated with CNO visited the noxious zones more frequently and for prolonged periods (FIGS. 2H to 2J, and FIG. 16). Similarly, inhibition of the BLA nociceptive ensemble eliminated pain affective-motivational behaviors induced by the optogenetic activation of peripheral primary afferent nociceptors (FIG. 17).

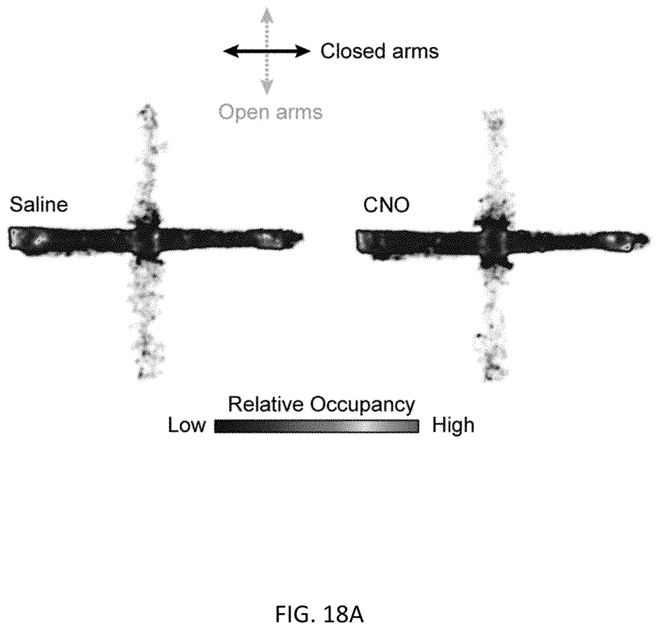

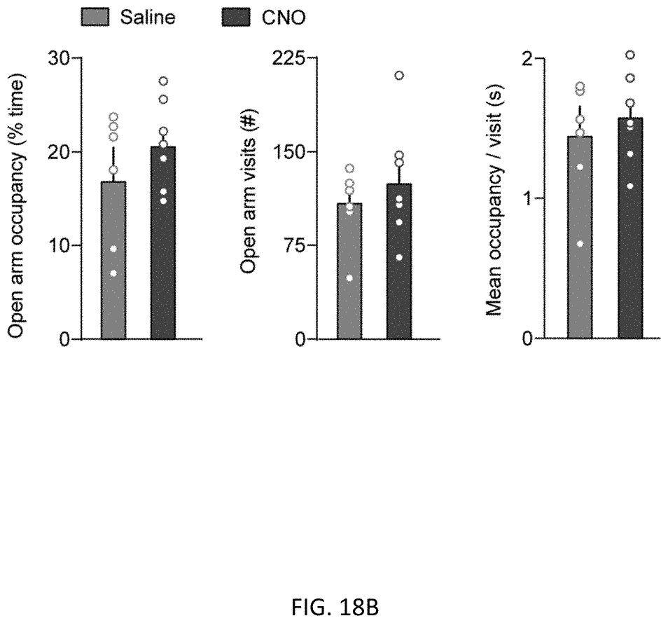

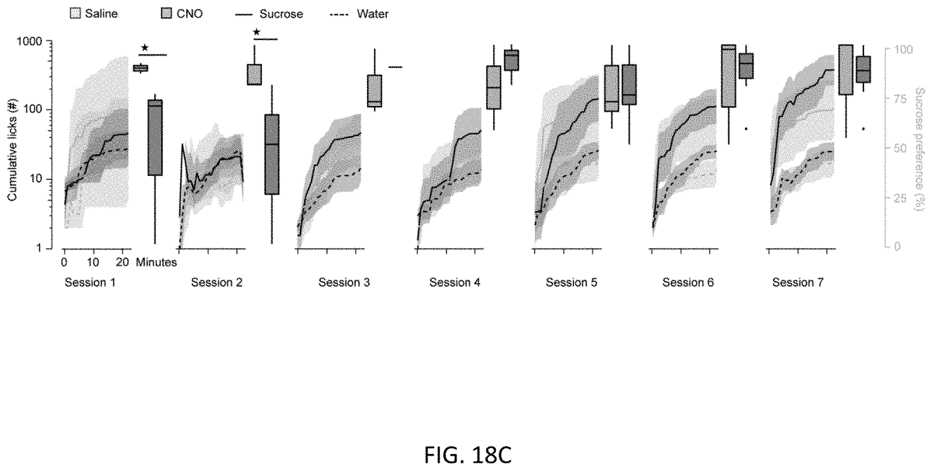

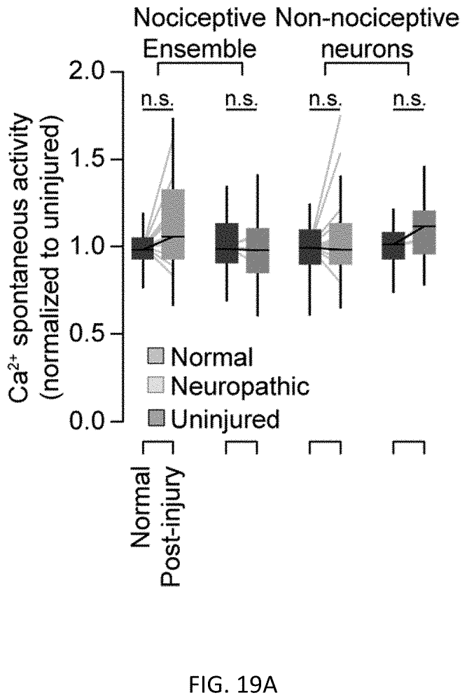

Whether pain and anxiety rely on common or distinct BLA ensembles is unknown; therefore, we placed noci-TRAP^{hM4} mice within an elevated plus maze, in which anxiety drives avoidance of the open arms (FIG. 2K). The noci-TRAP^{hM4} mice given either saline or CNO displayed equivalent visits to and occupancy of the open arms (FIGS. 18A and 18B). Since nociceptive and sucrose reward-related information were encoded in divergent networks (FIG. 1J), we tested the contribution of the nociceptive ensemble to appetitive motivational drive during sucrose preference training. CNO enhanced sucrose reward in sucrose-naïve conditions (28) but had no retarding effects on preference development or on lick rates, relative to controls (FIG. 2L and FIG. 18C). Thus, this BLA nociceptive ensemble transforms emotionally inert nociceptive information into an affective signal that is necessary for the selection and learning of motivational protective pain behaviors.

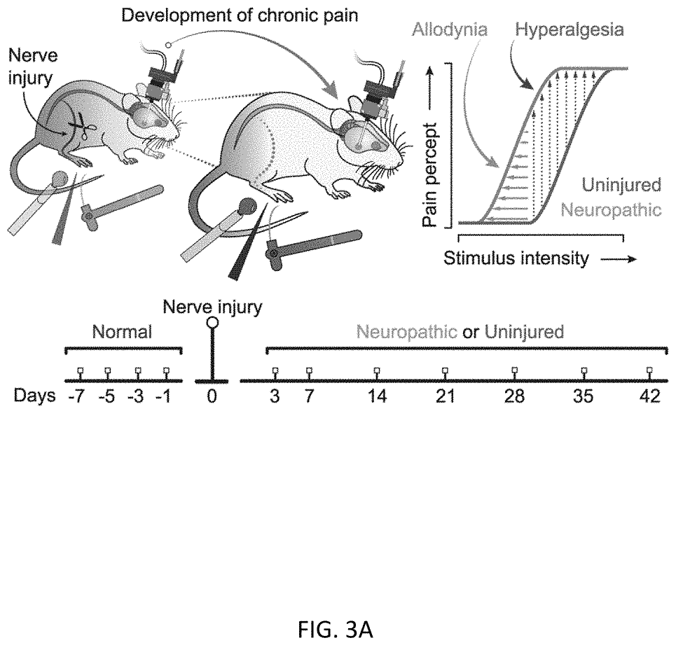

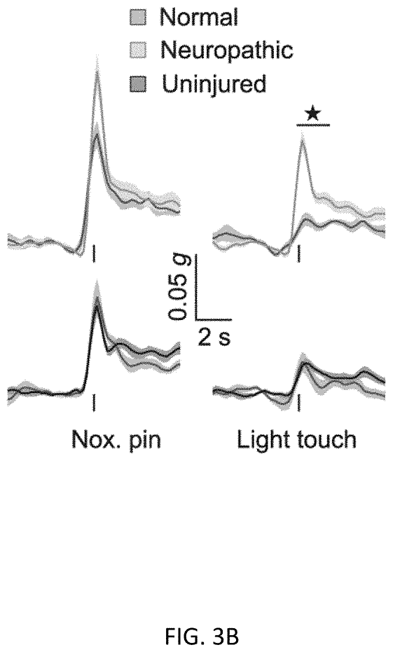

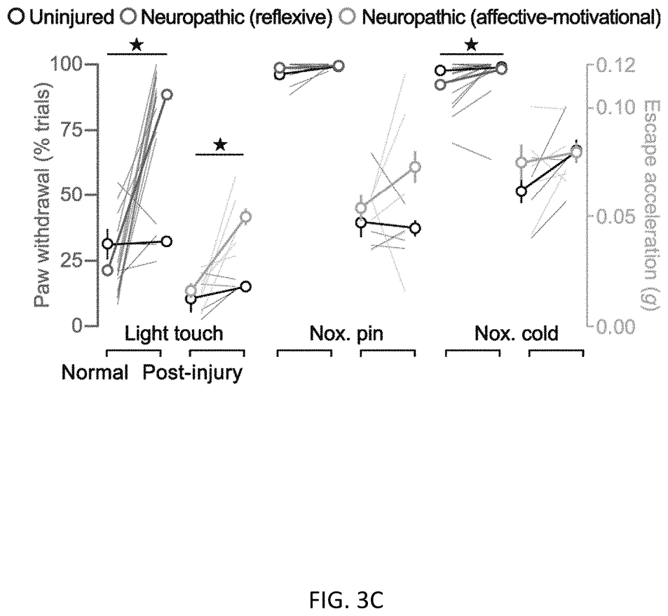

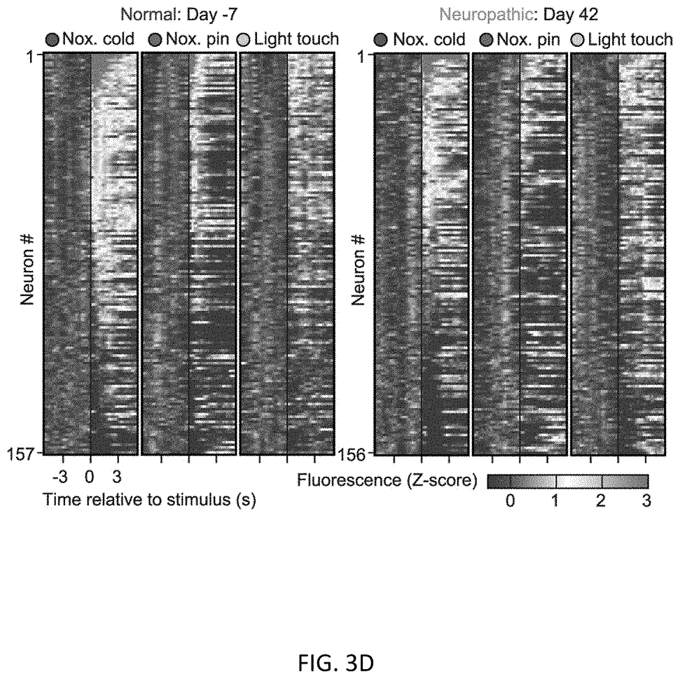

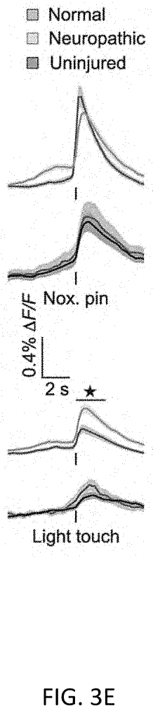

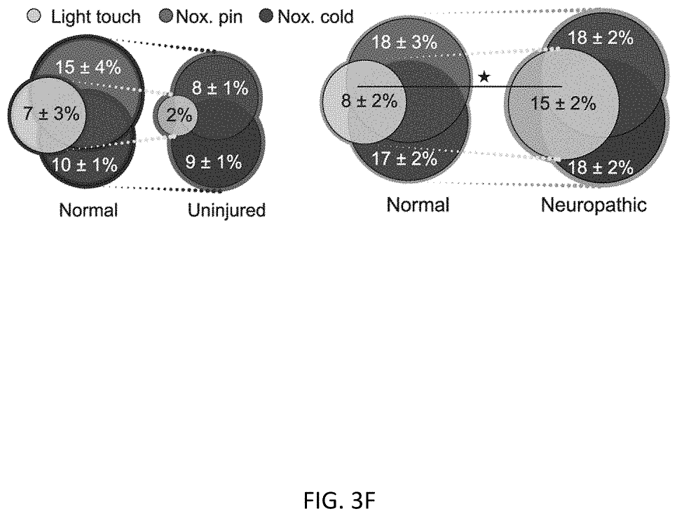

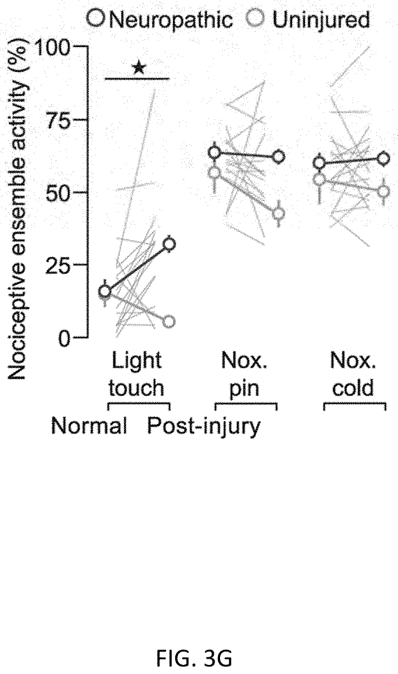

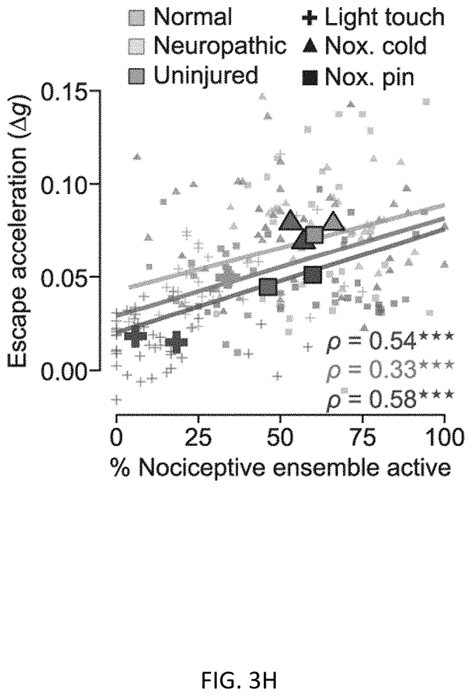

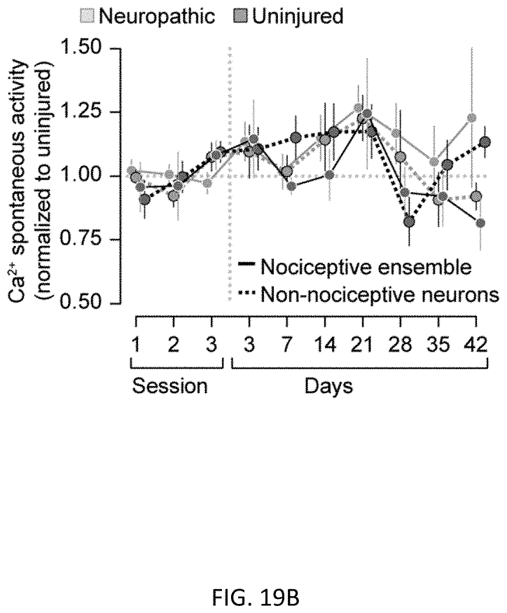

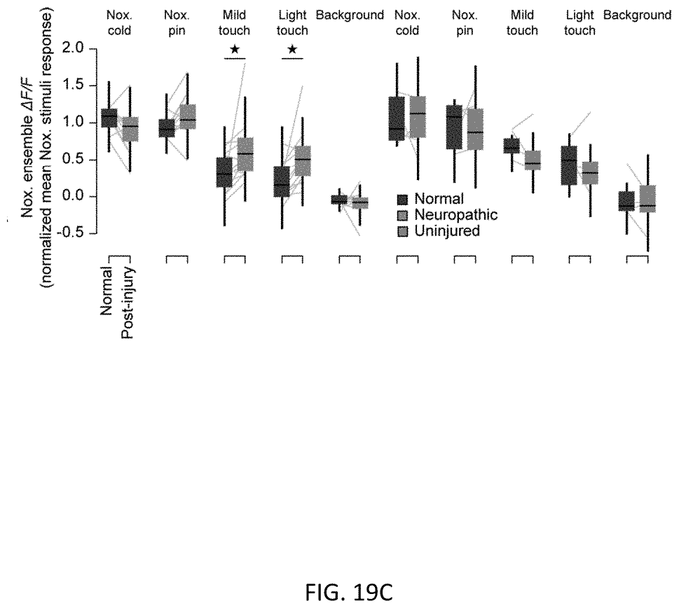

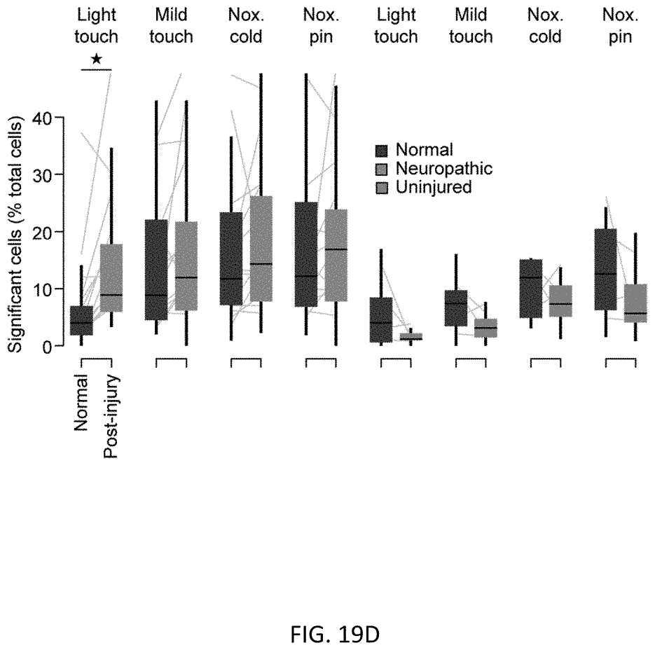

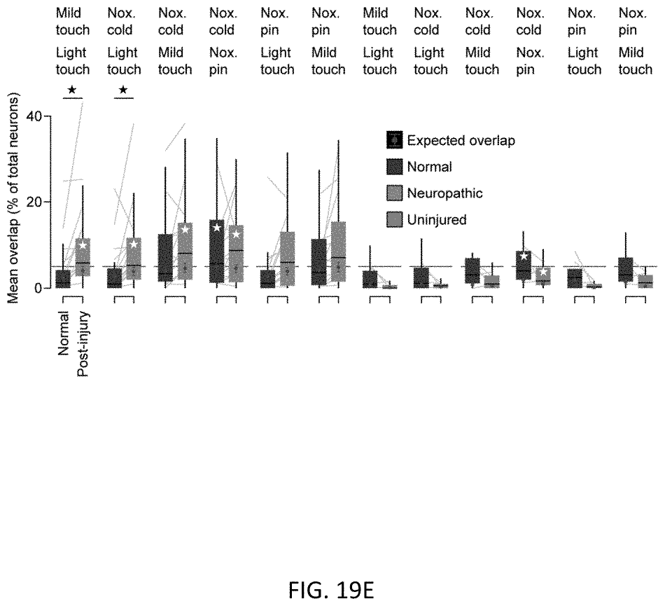

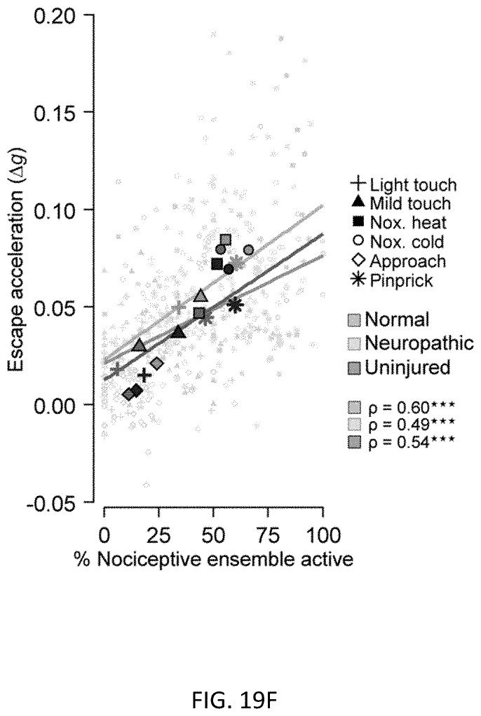

We next investigated the contribution of BLA neural ensemble activity to chronic pain. A hallmark of chronic neuropathic pain is the appearance of allodynia and hyperalgesia, both pathological perceptual states in which aversion is ascribed to innocuous somatosensory stimuli and exacerbated in response to noxious stimuli, respectively (FIG. 3A) (29). We hypothesized that this pathological perceptual switch might result from maladaptive transformations in BLA coding. We tracked the longitudinal dynamics of BLA ensembles before and after the development of neuropathic pain induced by sciatic nerve injury (17,396 neurons, n=17 mice) (FIG. 3). Throughout the development of chronic neuropathic pain, a subset of neurons stably encoded the nociceptive ensemble for both noxious mechanical and cold stimuli (FIG. 10). Nerve injury did not significantly increase the spontaneous activity of the nociceptive ensemble and overall BLA population (FIGS. 19A and 19B). However, BLA neural activity elicited in response to light touch displayed a significant expansion within the nociceptive ensemble in neuropathic (291±88% increase) but not in uninjured mice (38±14% decrease) (FIGS. 3D to 3G, and FIGS. 19, 19C to 19E). The emergence of this neuropathic coding schema was accompanied by the development of reflexive paw withdrawal hypersensitivity and by enhanced affective-motivational pain behaviors (FIGS. 3B and 3C, and FIGS. 8C to 8F). The magnitudes of the behavioral responses and the BLA nociceptive ensemble Ca² activity were significantly correlated before and after injury (FIG. 3H and FIG. 19F). These results suggest a role for the BLA in the emergence of allodynia in chronic pain states.

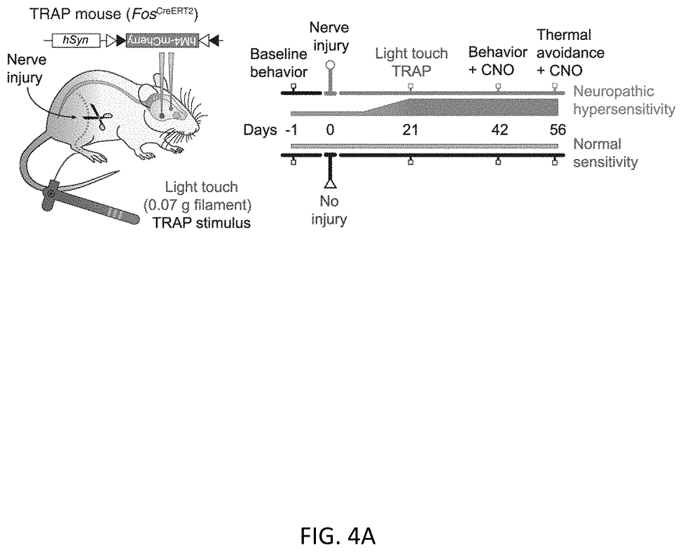

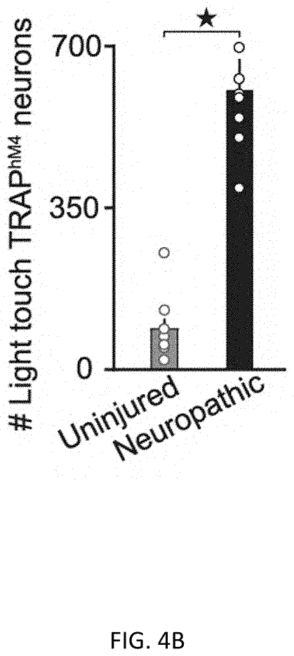

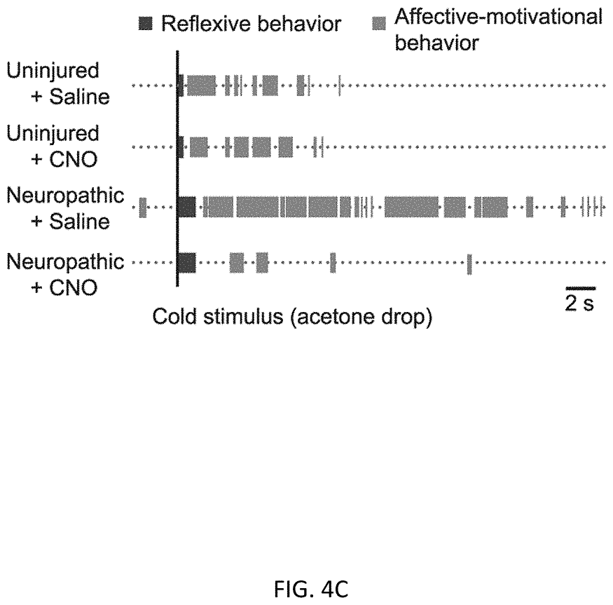

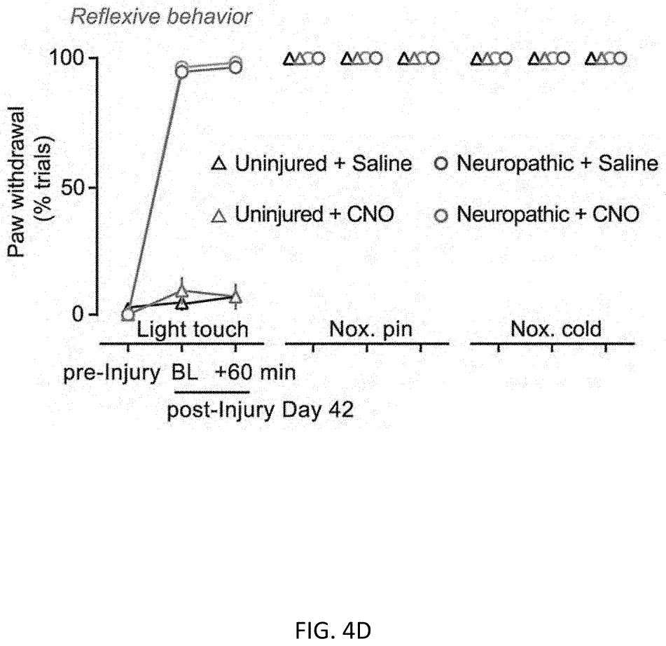

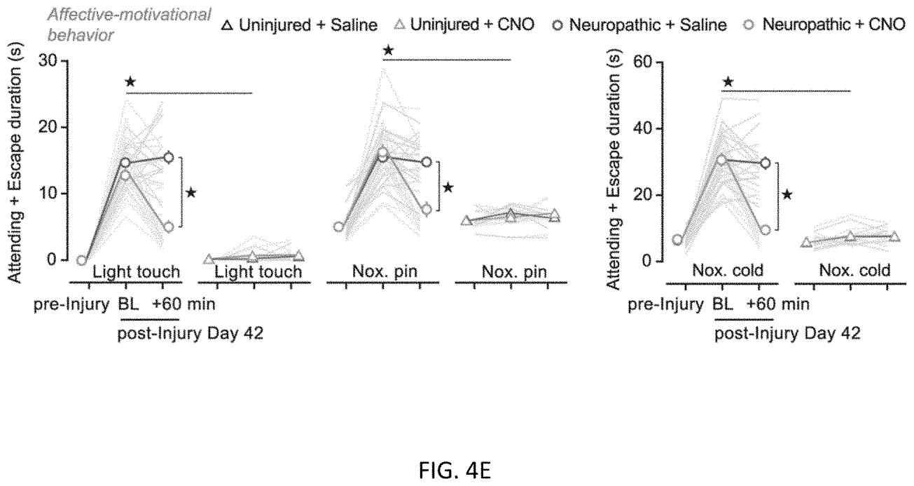

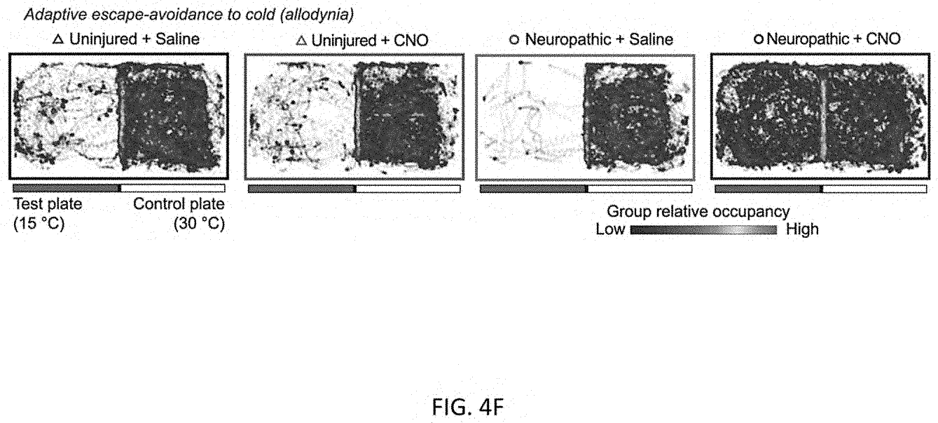

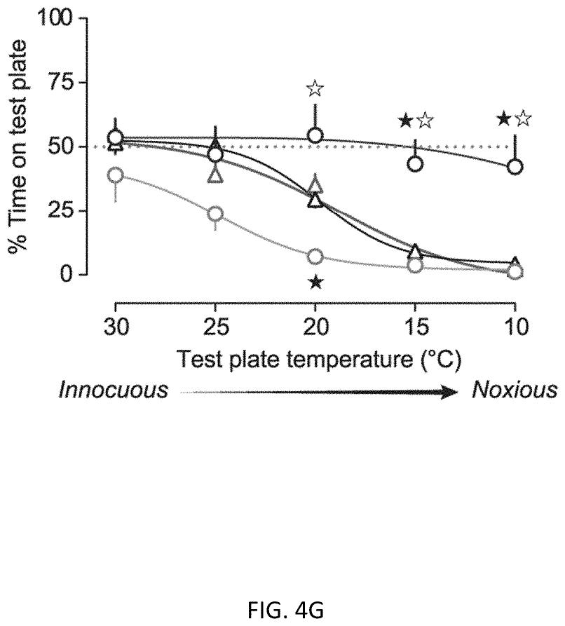

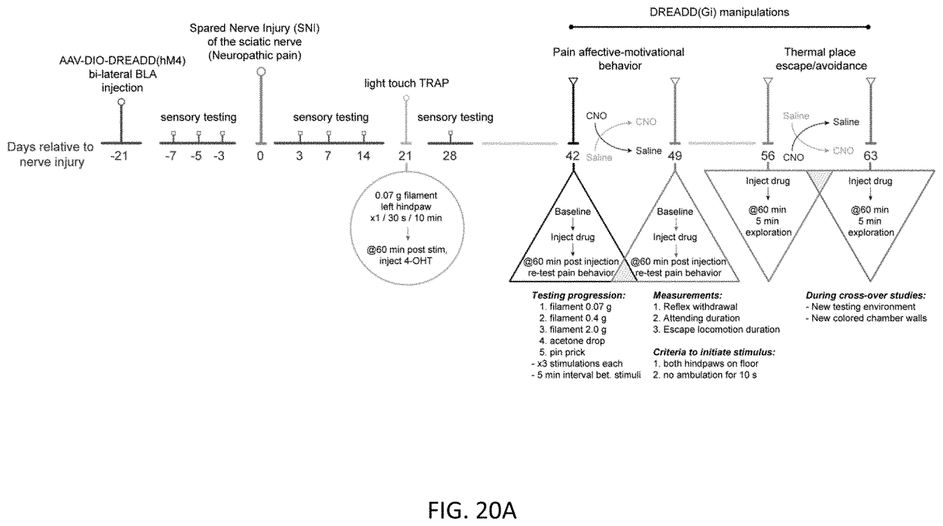

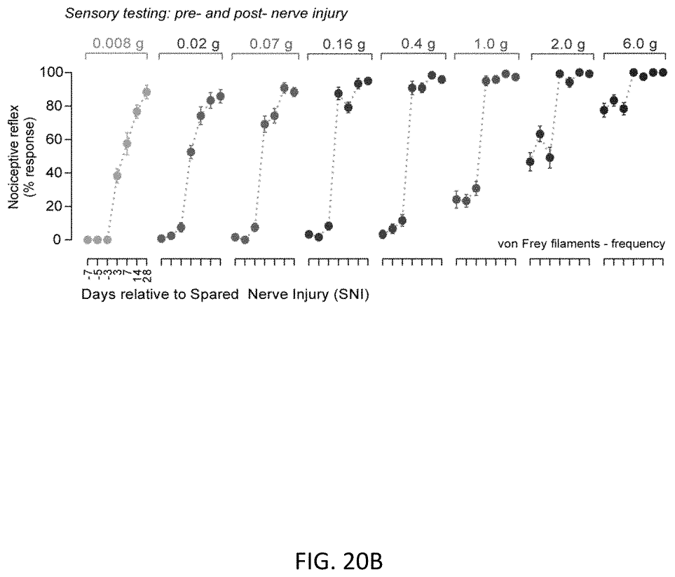

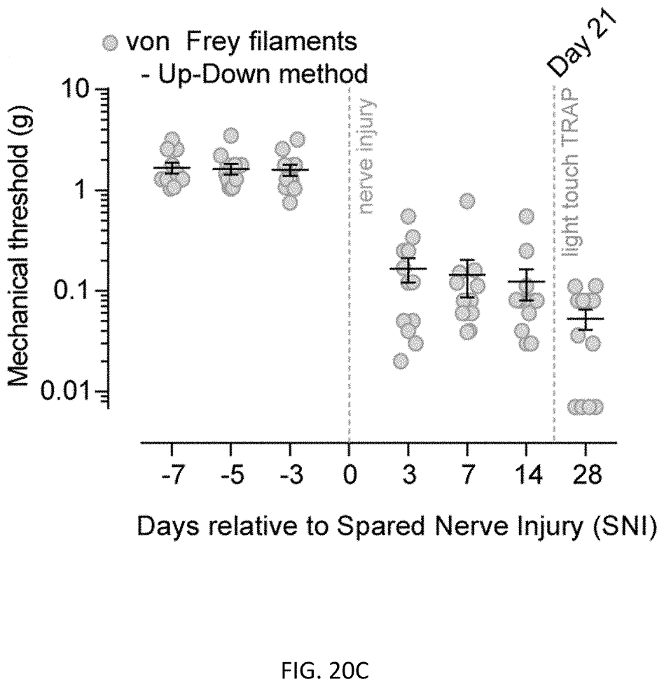

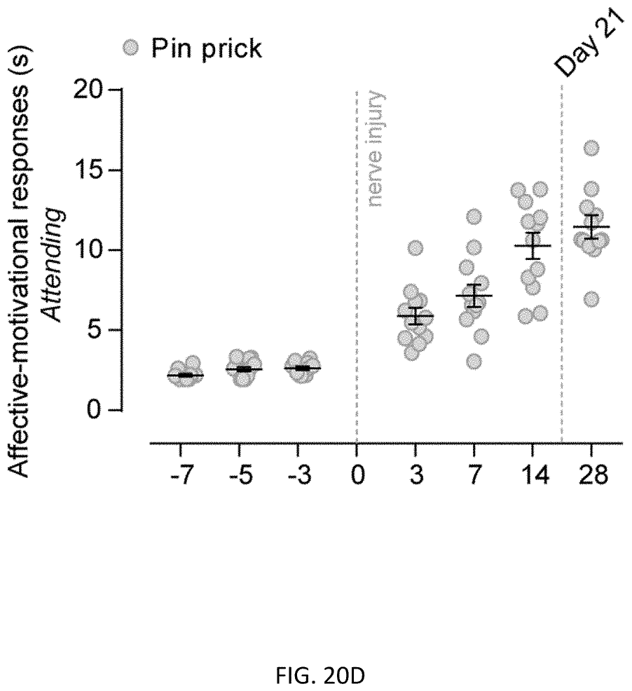

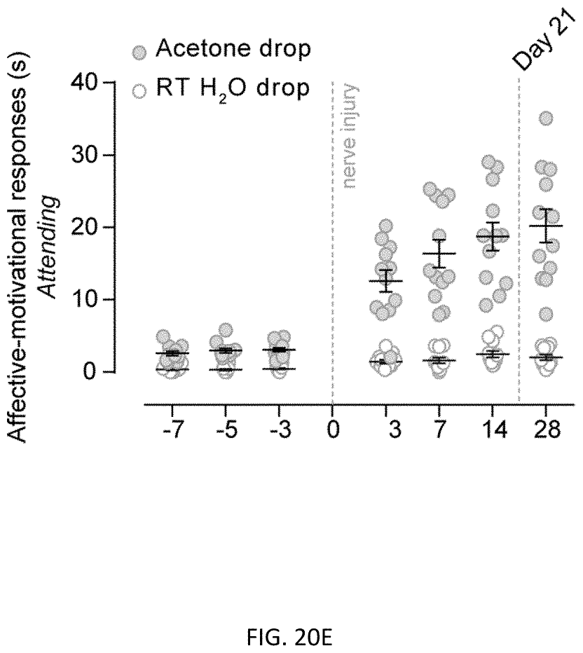

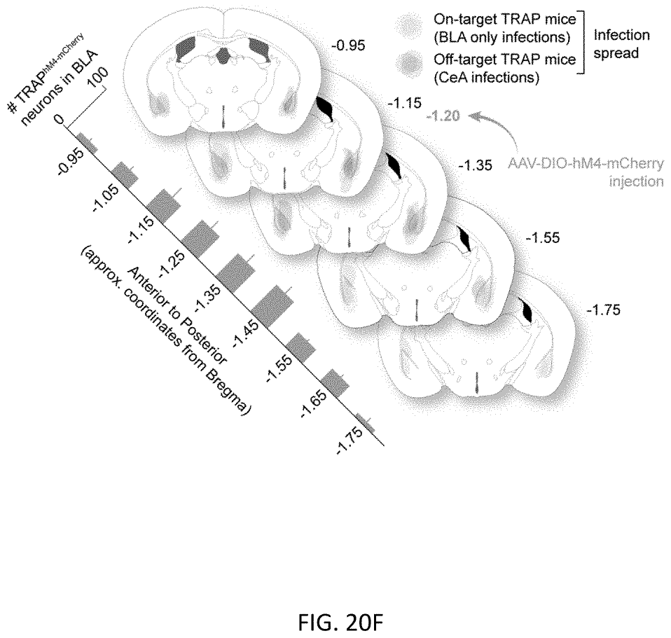

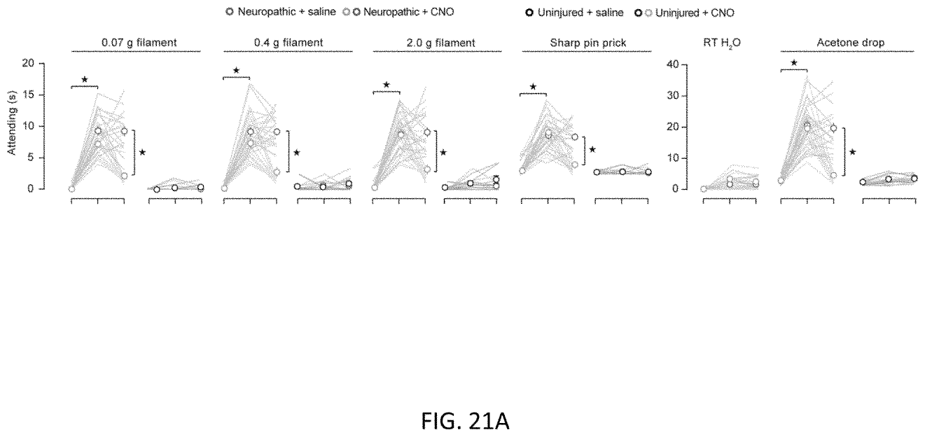

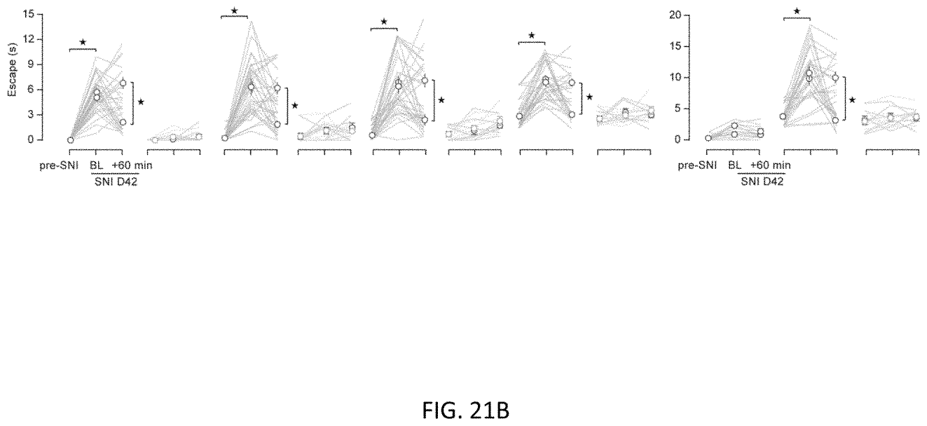

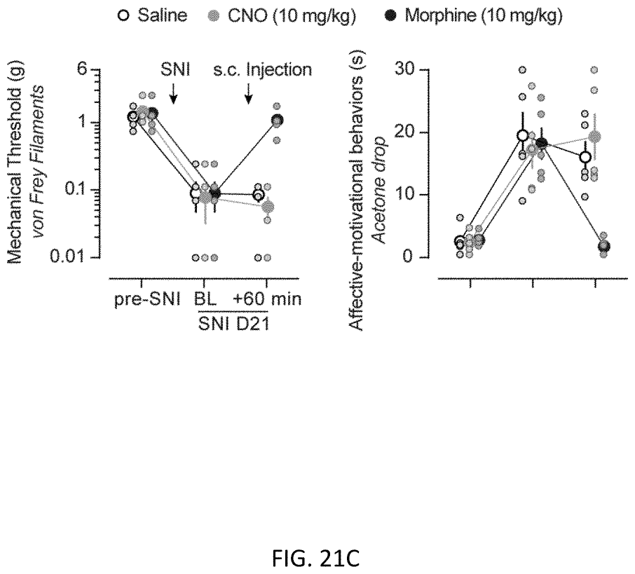

We next asked if we could prevent the neural transformation of light touch sensory information into an aversive signal and eliminate chronic pain unpleasantness by gaining genetic access to the nociceptive ensemble with innocuous stimuli in neuropathic TRAP mice. At 21 days post-nerve injury, when allodynia had fully developed (FIGS. 20B to 20E), we delivered a light touch TRAP protocol to express hM4-mCherry in the BLA nociceptive ensemble (neuropathic TRAP^{hM4} mice) (FIGS. 4A and 4B, and FIG. 20). At day 42 postinjury, neuropathic TRAP^{hM4} mice displayed significant allodynia and hyperalgesia, for both reflexive and affective-motivational pain responses, relative to uninjured mice (FIGS. 4C to 4E). While the injection of CNO in neuropathic TRAP^{hM4} mice did not alter reflexive hypersensitivity (FIG. 4D), we observed a profound decrease in neuropathic affective-motivational behaviors, regardless of stimulus intensity or modality (FIG. 4E and FIGS. 21A and 21B). Uninjured TRAP^{hM4} mice given the light touch TRAP protocol expressed levels of hM4-mCherry in the BLA that were similar to those of non-stimulated control mice (FIG. 4B and FIG. 2C), presumably because the nociceptive ensemble does not strongly encode innocuous information under normal conditions (FIG. 1I). We observed neither CNO-mediated changes in affective-motivational pain behaviors in these uninjured mice nor CNO effects on neuropathic reflexive or affective-motivational behaviors in the absence of hM4 expression (FIGS. 4C to 4E, and FIGS. 21A and 21B). In addition to tactile allodynia, patients with neuropathic pain often report intense pain in response to cold temperatures (cold allodynia).

We therefore ran neuropathic TRAP^{hM4} mice through a two-chamber thermal escape-avoidance assay in which the floor of one chamber was cooled (from 30° to 10° C.) (FIG. 4F). Uninjured TRAP^{hM4} mice avoided the cold chamber, while mice with nerve injury showed enhanced avoidance, consistent with allodynia (FIGS. 4F and 4G).

Notably, CNO administration to neuropathic TRAP^{hM4} mice generated a near-total indifference between cold and neutral temperature chambers (FIGS. 4F and 4G). Together, these results indicate that the BLA nociceptive ensemble is also necessary for the pain aversion associated with allodynia and hyperalgesia during chronic pain states.

Thus, disrupting neural activity in a nociceptive ensemble in the BLA is sufficient to reduce the affective dimension of pain experiences, without altering their sensory component. The unconditioned nociceptive ensemble described here is a stable network of amygdalar principal neurons that is responsive to a diverse array of noxious stimuli. Within this ensemble, combinatorial neural ensemble codes distinguish the various thermal and mechanical nociceptive stimuli. These codes likely represent stimulus modality, intensity, salience, and valence to provide additional qualitative information that enriches individual pain affect percepts (30). The presence of a purely nociceptive-specific subpopulation of neurons within the larger BLA nociceptive ensemble, distinct from general aversion-encoding populations, suggests the capacity for computing and assigning an accompanying “pain tag” to valence information. This categorical signal could prioritize the negative valence of intense noxious stimuli and scale the selection of conative pain protective behaviors. It is thought that hierarchical pathways transform low-level sensory inputs into higher-order affective responses (5, 31). Our chemogenetic manipulations suggest that this critical node in the nociceptive brain circuitry plays a critical role in shaping pain experiences, by providing an evaluation of nociceptive information that, in turn, intrinsically motivates protective behaviors associated with pain (32).

The phenomenological description of a pain experience is normally that of a complex but unified sensory and emotional perception that can neither exist alone as an unanchored aversive state nor stand merely on its emotionally inert sensory qualities (33, 34). Though activity within the BLA nociceptive ensemble cannot account for the instantiation of the entire pain experience, we propose that the BLA nociceptive ensemble transmits abstracted valence information to the central amygdala, striatal, and cortical networks (35-37). For example, BLA neurons projecting to the CeA may send a “pain tag” that helps select for appropriate defensive responses to impending or immediate threats (23). In parallel, connected cortical regions might coalesce BLA affective signals with sensory-discriminative information to process them against prior experiences and internal states for further evaluation at cognitive levels, all of which contribute to the construction of a pain experience (4, 38).

During chronic pain states, BLA ensemble coding of innocuous somatosensory information changes to engage the nociceptive ensemble, leading to perceived aversion and protective behavioral responses when encountering normally nonpainful stimuli, such as light touch. Whether this change in ensemble activity results from peripheral or central sensitization (3, 39), amygdalar input, or intra-amygdala plasticity (11) remains an open question. Chronic pain is not simply a sensory disorder but a neurological disease with affective dysfunction that profoundly impacts the mental state of millions of pain patients (40). Clinical management of chronic pain remains a staggering challenge, given the heterogeneity of underlying causes, and the overreliance on opioid analgesics has contributed to the opioid epidemic (41, 42). Comprehensive strategies that provide substantive relief across pain types are urgently needed (43). To make progress along this translational path, we have identified in the BLA a critical neural ensemble target that mediates chronic pain unpleasantness. This finding may enable the development of chronic pain therapies that could selectively diminish pain unpleasantness, regardless of etiology, without influencing reward, and importantly, preserving reflexes and sensory-discriminative processes necessary for the detection and localization of noxious stimuli (44, 45). Collectively, our findings begin to refine the neural basis and coding principles underlying the multiple dimensions and complexity of the pain experience for developing more effective analgesic therapies.

### REFERENCES

1. D. D. Price, Science 288, 1769-1772 (2000).
2. M. N. Baliki, A. V. Apkarian, Neuron 87, 474-491 (2015).
3. C. Peirs, R. P. Seal, Science 354, 578-584 (2016).
4. L. Garcia-Larrea, H. Bastuji, Prog. Neuropsychopharmacol. Biol. Psychiatry (2017).
5. P. H. Janak, K. M. Tye, Nature 517, 284-292 (2015).
6. N. Hebben, S. Corkin, H. Eichenbaum, K. Shedlack, Behav. Neurosci. 99, 1031-1039 (1985).
7. N. Neimann et al., Bull. Soc. Fr. Dermatol. Syphiligr. 71, 292-294 (1964).
8. M. Ploner, H. J. Freund, A. Schnitzler, Pain 81, 211-214 (1999).
9. M. L. Uhelski, M. A. Davis, P. N. Fuchs, Pain 153, 885-892 (2012).
10. M. Costigan, J. Scholz, C. J. Woolf, Annu. Rev. Neurosci. 32,1-32 (2009).
11. V. Neugebauer, Amygdala Pain Mechanisms. Handb. Exp. Pharmacol. 227, 261-284 (2015).
12. P. C. Chang, M. V. Centeno, D. Procissi, A. Baria, A. V. Apkarian, Pain 158, 488-497 (2017).
13. L. E. Simons et al., The responsive amygdala: Treatment-induced alterations in functional connectivity in pediatric complex regional pain syndrome. Pain 155, 1727-1742 (2014).
14. J. A. Hashmi et al., Brain 136, 2751-2768 (2013).
15. F. Gore et al., Cell 162, 134-145 (2015).
16. V. Neugebauer, W. Li, G. C. Bird, G. Bhave, R. W. Gereau 4th, J. Neurosci. 23, 52-63 (2003).
17. S. Han, M. T. Soleiman, M. E. Soden, L. S. Zweifel, R. D. Palmiter, Cell 162, 363-374 (2015).
18. G. Ji et al., J. Neurosci. 30, 5451-5464 (2010).
19. B. F. Grewe et al., Nature 543, 670-675 (2017).
20. Y. Ziv et al., Nat. Neurosci. 16, 264-266 (2013).
21. J. G. Parker et al., Nature 557, 177-182 (2018).
22. W. Zhang et al., J. Neurosci. 33, 722-733 (2013).
23. P. Namburi et al., Nature 520, 675-678 (2015).
24. J. Kim, M. Pignatelli, S. Xu, S. Itohara, S. Tonegawa, Antagonistic negative and positive neurons of the basolaterial amygdala. Nat. Neurosci. 19, 1636-1646 (2016).
25. C. J. Guenthner, K. Miyamichi, H. H. Yang, H. C. Heller, L. Luo, Neuron 78, 773-784 (2013).
26. B. L. Roth, Neuron 89, 683-694 (2016).
27. J. L. Gomez et al., Science 357, 503-507 (2017).
28. G. G. Calhoon et al., Acute food deprivation rapidly modifies valence-coding microcircuits in the amygdala. bioRxiv 285189 [Preprint]. 19 Mar. 2018.
29. C. A. von Hehn, R. Baron, C. J. Woolf, Neuron 73, 638-652 (2012).
30. B. W. Balleine, S. Killcross, Trends Neurosci. 29, 272-279 (2006).
31. C. D. Salzman, S. Fusi, Annu. Rev. Neurosci. 33, 173-202 (2010).
32. J. E. LeDoux, R. Brown, Proc. Nati. Acad. Sci. U.S.A. 114, E2016-E2025 (2017).
33. N. Grahek, Feeling Pain and Being in Pain (MIT Press, 2007).
34. R. Melzack, K. Casey, in The Skin Senses (Charles C. Thomas, 1968), pp. 432-439.
35. F. Ramirez, J. M. Moscarello, J. E. LeDoux, R. M. Sears, J. Neurosci. 35, 3470-3477 (2015).
36. A. Burgos-Robles et al., Amygdala inputs to prefrontal cortex guide behavior amid conflicting cues of reward and punishment. Nat. Neurosci. 20, 824-835 (2017).
37. A. Beyeler et al., Cell Reports 22, 905-918 (2018).
38. K. Wiech, Science 354, 584-587 (2016).
39. C. Torsney, A. B. MacDermott, J. Neurosci. 26, 1833-1843 (2006).
40. P. A. Pizzo, N. M. Clark, O. Carter Pokras, Relieving Pain in America: A Blueprint for Transforming Prevention, Care, Education, and Research (National Academies Press, 2011).
41. N. D. Volkow, F. S. Collins, N. Engl. J. Med. 377, 391-394 (2017).
42. G. Corder, D. C. Castro, M. R. Bruchas, G. Scherrer, Endogenous and exogenous opioids in pain. Annu. Rev. Neurosci. 41, 453-473 (2018).
43. P. Skolnick, N. D. Volkow, Neuron 92, 294-297 (2016).
44. F. Jelasid, Confin. Neurol. 27, 53-55 (1966).
45. T. Grosser, C. J. Woolf, G. A. FitzGerald, Science 355, 1026-1027 (2017).
46. B. Ahanonu, calciumlmagingAnalysis: A software package for analyzing one- and two-photon calcium imaging datasets. GitHub (2018); doi.org/10.5281/zenodo.2222295.

### Example 2

### Materials and Methods

### Animals

All procedures were approved by the Stanford University Administrative Panel on Laboratory Animal Care in accordance with American Veterinary Medical Association guidelines and the International Association for the Study of Pain. We housed mice 1-5 per cage and maintained them on a 12-hour light/dark cycle in a temperature-controlled environment with ad libitum access to food and water. Animals undergoing active Ca imaging experiments (after mounting the miniature microscope baseplate) were singly housed. For behavioral manipulation and neuroanatomy experiments, we utilized Fos-CreERT2 mice (B6.129(Cg)-Fostml.1(cre/ERT2) Luo/J, Jackson Laboratory, stock #21882, male, aged 8-15 weeks at the start of all experiments). For BLA miniature microscope imaging experiments, we utilized C57BV/6J mice (Jackson Laboratory, stock #664, male, aged 8-12 weeks at the start of experiments). For dorsomedial striatum (DMS) miniature microscope imaging experiments, we utilized wild-type (Shank3B^{+/+}) or knockout (Shank3B^{-/-}) Shank3B;Drd1a^{Cre/+} or Shank3B;A2A^{Cre/+}. mice obtained from Guoping Feng (MIT).

### Drugs

4-hydroxytamoxifen (Sigma, #H6278) prepared in Kolliphor EL (Sigma, #27963), Clozapine-N-oxide (Tocris, #4936), and 0.9% Sodium chloride (Hospira, #NDC 0409-4888-10).

### Viral Reagents

### Viral Reagents for Miniature Microscope Imaging

For Ca²⁺ imaging using GCaMP6m (68) in BLA Camk2a⁺ principal neurons, we intracranially injected 500 nL of AAV2/5-Camk2a-GCaMP6m-WPRE (Schnitzer lab custom preparation; titre: 6.7×10¹² GC/mL for FIGS. 7A, 7C mice) into the right BLA at coordinates anteroposterior (AP): −1.60 mm, mediolateral (ML): +3.32 mm, dorsoventral (DV): −4.70 mm (FIG. 7B animals) or AP: −1.70 mm, ML: +3.30 mm (−3.30 mm for left BLA mice), DV: −4.70 mm (FIGS. 7A, 7C animals). For Ca²⁺ imaging in DMS D1 or D2 dopamine receptor-expressing medium spiny neurons, we injected mice with AAV2/9-CAG-FLEX-GCaMP6m (Schnitzer lab custom preparation; titre: 1.37×10¹² GC/mL) at coordinates AP: −0.80 mm, ML: +1.50 mm, DV: −2.5 mm (down to −3.0 then back up to −2.5 mm from dura).

### Viral Reagents for TRAP Studies

For chemogenetic activity manipulation of BLA neuronal ensembles, we intracranially injected 100 nL of either AAV5-hSyn-DIO-hM4-mCherry (U. North Carolina Viral Core; titre: 3.98×1012), AAV5-hSyn-DIO-mCherry (U. North Carolina Viral Core; titre: 4.72×1012), AAVDJ-Ef1a-DIO-eYFP (Stanford Viral Core; titre: 2.65×1011) into both the left and right BLA at coordinates AP: −1.20 mm, ML: ±3.1 mm, DV: −4.60 mm.

For transdermal optogenetic activation of primary afferent nociceptors, we intrathecally injected 2.5 μL of AAV6-hSyn-ChR2(H134R)-eYFP (U. North Carolina Viral Core; titre: 2.17×1013) directly into the subarachnoid space so that the virus reaches the CSF and can infect nociceptors.

### Stereotaxic Injections and Surgical Procedures

### Injection Procedures for TRAP Study Animals

We conducted all surgeries under aseptic conditions using a digital small animal stereotaxic instrument (David Kopf Instruments). We anaesthetized mice with isoflurane (5% induction, 1-2% maintenance) in the stereotaxic frame for the entire surgery and maintained their body temperature using a heating pad. We injected mice with a beveled 33G needle facing medially, attached to a 10-μL microsyringe (Nanofil, WPI) for delivery of viral reagents at a rate of 20 nL/min for more precise targeting (e.g., of DREADD (hM4) expression) using a microinjection unit (Model 5000, Kopf). After reagent injection, the needle was raised 100 μm for an additional 10 min to allow the virus to diffuse at the injection site and then slowly withdrawn over an additional 3 min. After surgery, we maintained the animal's body temperature using a radiant heat lamp until fully recovered from anesthesia.

### Injection Procedures for Miniature Microscope Animals

We conducted all surgeries under aseptic conditions with glass bead sterilized surgical tools (Dent-Eq, BS500) and used a digital small animal stereotaxic instrument (David Kopf Instruments). We anaesthetized mice with isoflurane (2-5% induction, 1-2% maintenance) in the stereotaxic frame for the entire surgery and maintained body temperature using a heating pad (FHC, DC Temperature Regulation System). For FIGS. 7A, 7C mice, we injected using a beveled 33G needle (WPI, NF33FBV-2), facing medially, attached to a 10-μL microsyringe (Nanofil, WPI). We delivered viral reagents at a rate of 250 nL/min using a microsyringe pump (UMP3, WPI) and its controller (Micro4, WPI) for GCaMP expression. We performed injections for FIG. 7B mice as described previously (19). After reagent injection, we raised the needle 100 μm for an additional 5-10 min to allow the virus to diffuse at the injection site and then slowly withdrew the needle over an additional minute. After surgery, animals recovered from anesthesia on a heating pad to maintain body temperature.

### Microendoscope Implantation and Mounting

### Microendoscope Implantation in BLA and DMS Mice

For BLA-implanted mice run through the protocol in FIGS. 7A, 7C and DMS-implanted mice in FIGS. 11A-11E, we performed stereotaxic implantation of a stainless steel cannula 11-19 days after AAV viral injections. We fabricated 1.06-mm-diameter stainless steel cannulas (custom cut 18G McMaster's 89935K66 to 4.2 mm length pieces at Stanford Varian Physics Machine Shop or ordered custom cut 304 S/S Hypodermic Tubing 18G to 4.3 mm length pieces from Ziggy's Tubes and Wires) and attached 2-mm-diameter 0.1-mm-thick Schott Glass (TLC International, custom order) onto one end using optical adhesive (Norland Optical Adhesive No. 81, NC9586074). We ground down the excess glass using a polisher (Ultra Tec ULTRAPOL End & Edge Polisher, #6390) and film (Ultra Tec, M.8228.1), and then placed the completed cannula in a sealed scintillation vial until use during implantation surgeries.

For implantation surgeries, we anaesthetized mice with isoflurane (2-5% induction, 1-2% maintenance, both in oxygen) and maintained their body temperature using a heating pad (FHC, DC Temperature Regulation System). After head hair removal (Nair, Church and Dwight Co. NRSL-22339-05) and opening the mouse skin, we performed small craniotomies in three locations—ML: (−0.7, 2.1, −3.1) mm and AP: (5.2, −3.6, −3.6) mm. We screwed three stainless steel screws (Component Supply Company, MX-000120-01SF) into the skull right up to dura and then performed a craniotomy using a drill (Osada Model EXL-M40) and 1.4 mm round drill burr (FST, 19007-14). We cleaned away bone fragments and other detritus from the opening using sterilized forceps (Fine Science Tools, Dumont #5 Forceps, 11252-20). We continuously applied mammalian Ringers (Fisher Scientific, 50-980-246) to the surgical area when necessary for the remainder of the surgery. To prevent increased intracranial pressure and improve quality of the imaging site, we aspirated all overlying tissue down to ˜DV: −4.20 mm (BLA mice) or −2.10 mm (DMS mice) with a 27G needle (Sai-Infusion, B27-50-27G or VWR Cat. No. 89134-172).

We attached a 1.06-mm stainless steel cannula onto a custom designed 3D printed cannula holder (Stratasys Objet30 printer, VeroBlackPlus material). For BLA-implanted mice, we lowered the cannula to AP: −1.70 mm, ML: +3.30 mm (right BLA) or −3.30 mm (left BLA), DV: −4.50 mm. For DMS-implanted mice, we lowered the cannula to AP: −0.80 mm, ML: +1.50 mm, DV: −2.35 mm. This placed the cannula ˜100-300 μm above the imaging plan based on the specifications of the GRIN lens microendoscope's imaging side working distance. Next, we immediately retracted the cannula from the craniotomy site and aspirated any additional debris or blood that had been pushed down during the initial implant then relowered the cannula into the implant site, covered the cannula with adhesive cement (C&B, S380 Metabond Quick Adhesive Cement System), and allowed the cement to fix for 2-3 min. We placed custom designed laser cut headbars (LaserAlliance, 18-24G thickness stainless steel) over the left posterior skull screw and applied alayer of dental cement (Coltene Whaledent, Hygenic Perm) to affix both the headbar and cannula to the skull. The cement dried for 7-10 min before we covered the cannula with bio tape (NC9033794 Tegaderm Transparent Dressing), fixed the tape to the cement with ultraviolet (UV) glue (Loctite® Light-Activated Adhesive #4305), and allowed the animal to recover from anesthesia on a heated pad.

### Verification of Microendoscope Implantation and GCaMP Expression in Awake, Behaving Mice

Several weeks after implantation, we checked awake animals for GCaMP6m fluorescence and Ca²⁺ transient activity on a custom designed apparatus. We avoided using anesthesia as this causes the BLA to exhibit reduced activity or become silent, which might have potentially led us to classify animals incorrectly as unusable due to lack of neural activity even though their neurons might have been active if the animal had been awake. We head-fixed mice by clamping (Siskiyou, CC-1) their headbar and allowed them to run on a running wheel (Fisher Scientific, InnoWheel, Catalog No. 14-726-577), which was attached via a custom designed 3D printed part (Stratasys Objet30 printer, VeroBlackPlus material) to a rotary encoder (Signswise 600P/R Incremental Rotary Encoder). We then lowered a custom-designed 1.0-mm-diameter microendoscope probe based on a gradient refractive index (GRIN) lens (Grintech GmBH) into the stainless steel cannula using forceps or a 27G needle attached to a vacuum line. We attached the miniature microscope onto a holder (Inscopix, Gripper Part, ID: 1050-002199) connected to a goniometer (Thorlabs, GN1) that allowed us to tilt the miniature microscope in x-z and y-z planes. We connected the holder to a three-axis micromanipulator and used it to lower the miniature microscope until we were in the microendoscope's focal plane. To determine an optimal part of the microendoscope to image neural activity, we made minor position adjustments of the miniature microscope in the x-y plane using the micromanipulator. To ensure the entire field-of-view was in focus, we adjusted the miniature microscope's tilt relative to the microendoscope. We used the imaging software (Inscopix, nVista 2.0) to display incoming imaging frames in units of relative fluorescence changes (AF/F); this allowed us to observe Ca²⁺ transient activity in the awake behaving mice. We checked for time-locked responses to both auditory (e.g., clap) and sensory (e.g., tail pinch) stimuli, along with any signs of indicator overexpression (i.e., brightly fluorescent neurons lacking Ca²⁺ transient activity). Mice passing both tests moved onto mounting of the miniature microscope baseplate.

### Miniature Microscope Baseplate Mounting

In anesthetized (2% isoflurane in oxygen) mice that met the criteria described above, we fixed the microendoscope in place with UV curable epoxy (Loctite® Light-Activated Adhesive #4305) and stereotaxically lowered the miniature microscope, with the baseplate attached, toward the top of the microendoscope until the brain tissue was in focus. To ensure that the entire field-of-view was in focus, we used a goniometer (Thorlabs, GN1) to adjust the orientation of the miniature microscope until it was parallel to that of the microendoscope. To fix the baseplate onto the skull, we built alayer of blue-light curable composite (Pentron, Flow-It N11VI) from the dental cement on the mouse's skull toward, but not touching, the baseplate, followed by a layer of UV-curable epoxy (Loctite® Light-Activated Adhesive #4305) that affixed the baseplate to the composite. To prevent external light from contaminating the imaging field-of-view, we coated the outer layer of the composite and UV glue with black nail polish (OPI, Black Onyx NL T02). We attached a custom-designed cover (LaserAlliance, 16G thickness stainless steel) to the baseplate to protect the microendoscope. After surgery, mice recovered from anesthesia on a heating pad (FHC, DC Temperature Regulation System). For animals run through the protocol of FIG. 7B, we implanted the microendoscope and mounted the baseplate as described in (19); we had previously run these mice (n=8), and only these mice of FIG. 7B, through a behavioral and imaging protocol as described in (19).

### Integrated Miniature Microendoscope Imaging and Animal Test Procedure

### Miniature Microscope Behavioral Protocol

After mounting the miniature microscope onto a mouse and checking for adequate GCaMP6m expression, we habituated each mouse to the testing environment for at least three days prior to imaging. To preclude any emotional contagion between mice, we brought only one mouse into the isolated, light-, sound- and temperature-controlled testing environment. Further, we housed mice individually.

The experimental procedure for mice (n=9) analyzed in FIGS. 1 and 3 is described below. A general outline is shown in FIGS. 7A, 7C. The experimenter stayed in the testing environment throughout habituation to limit variations related to stress. The main protocol consisted of three or four imaging sessions performed on non-consecutive days (days −7, −5, −3, −1 or −6, −4, −2 pre-SNI) to allow animals to recover, and to reduce photobleaching resulting from long imaging sessions.

At the start of each imaging session, we head-fixed the mouse (using a Siskiyou CC-1), mounted the miniature microscope, checked for GCaMP6m fluorescence, aligned the field of view (FOV) to the previous session FOV, and placed the mouse within the test chamber. Before sensory stimulation, we measured spontaneous neural activity by recording Ca²⁺ activity for 10 min while the mouse habituated to, and freely moved within, the testing box. The mouse received no explicit experimenter-delivered sensory stimuli during this period. After baseline recording, the mouse had 15 min of access to an incentive (sucrose) to capture BLA neural responses to positive valence stimuli. To induce mice to lick without needing prior water deprivation, we used a 10% sucrose solution. We detected licks and delivered sucrose using a custom-built circuit based on a previous design (69). A custom electronic circuit (built using Arduino elements) collected lick data and synchronized all incoming data using output TTL pulses from the miniature microscope DAQ. Control signals from this circuit drove a solenoid (NResearch, 161P011) that delivered 10% sucrose instantly after the 1st lick in a bout. We programmed a 5-s-period between liquid deliveries. Thus, even if the mouse licked continuously, this approach provided a sufficient interval between incentive deliveries to relate the evoked neural activity with specific delivery time points. Next, the mouse began the sensory testing protocol, in which the experimenter delivered a battery of stimuli: 0.07-g and 1.4- or 2.0-g von Frey hairs (light and mild touch); 25G needle (noxious pin prick); water drops at 5° C. or acetone (noxious cold), 30° C. (innocuous liquid, for FIG. 7A animals [n=2]), and 55° C. (noxious heat) delivered via applying a small drop from a 1-mL syringe; fake-out stimuli where no contact was made (“Approach/No contact”); and noise (startle response control). We delivered all stimuli 15 times per session, except “Approach/No contact” and noise, which we delivered 9 times each. See FIG. 7 for details about timing information related to individual stimuli and stimuli blocks. We wrote custom code in the R computing environment to design a randomized stimulus delivery protocol for each session, subject to the following constraints: light touch, noxious cold, mild touch, and innocuous liquid or noxious heat had a set order at the beginning; the same stimuli could not have adjacent stimuli blocks; and “Approach/No contact” stimuli blocks would occur during the first 3 main stimuli super-blocks. We measured withdrawal reflexes and affective-motivational behaviors (attending and escape) using high-speed cameras (AVT Guppy Pro F-125 ⅓″ CCD Monochrome Camera #68-567 or The Imaging Source DMK 23FM021) and accelerometers (Sparkfun ADXL335 or ADXL345, with data collected using an Arduino Uno or Saleae Logic 8). We included “Approach/No contact” trials to detect possible BLA responses related to expectation of stimulus delivery and error-prediction. These “Approach/No contact” imaging trials consisted of bringing either a 0.07-g von Frey hair, a 25G needle, 1-mL syringe, or an 85-dB noise delivery device toward the animal but neither making contact nor turning on the noise. We randomly interspersed “Approach/No contact” trials between other stimuli blocks. To control for the possibility that the BLA hindpaw stimuli responses were startle-induced, we used a loud tone (˜80-85 dB) as an aversive but non-nociceptive sensory stimulus. We delivered the tone (centered around 4 kHz) for 300 ms by triggering an Arduino, loaded with custom code, to drive a TDK PS1240 Piezo Buzzer.

Subsequently, FIGS. 7A, 7C mice underwent a modified spared nerve injury (SNI) surgery (see ‘Chronic neuropathic pain model’ below for a description of the surgical procedure) (70). We then repeated the sensory testing protocol and recording of neural activity at 3, 7, 14, 21, 28, 35, 42 days post-surgery, as thermal and mechanical hypersensitivity developed and persisted (FIGS. 8E-8F). After SNI, mice started showing pain affective-motivational responses such as attending or escape behaviors when experimenters stimulated the injured paw with an innocuous 0.07-g von Frey hair (mechanical allodynia) or 5° C. water droplet (cold allodynia). At times, mice also displayed increased pain affective-motivational responses when stimulated with a sharp pin (mechanical hyperalgesia) or 55° C. water (heat hyperalgesia).

For mice given the procedure in FIG. 7B, a simplified protocol allowed assessment of the interaction between BLA neuron response to innocuous and noxious stimuli. Before stimulation, we measured background neural activity by recording Ca²⁺ activity for 15 min while each mouse freely explored a 17.78 cm×19.05 cm box. We then transferred each mouse to the behavior testing chamber (10.16 cm×15.24 cm) where it habituated for −5 min. We then delivered a battery of stimuli starting with three superblocks, in which 0.07-g (light touch), 0.4-g (moderate touch), and 2.0-g (mild touch) von Frey filament stimuli were each given ten times at intervals of 30 s with 60 s between stimuli blocks and 3 min between superb-locks. Next, we applied drops of acetone (noxious cold) 10 times at 60 s intervals followed by pricking the skin with a 25G needle (noxious pin) 10 times. Mice then underwent the SNI surgery and were imaged at days 3, 7, 14, and 21 post-surgery.

### Miniature Microscope Behavior Recording Hardware

For animals run through the procedure in FIGS. 7A, 7C, we synchronized all incoming data in the following manner. The miniature microscope acted as the master controller of event timing as we considered time locking to Ca²⁺ activity the most critical feature. We had two hardware setups for collecting all relevant behavior videos, stimulus delivery times, and accelerometer data: one relied on a set of Arduino microcontrollers and the other one used two Saleae Logic 8 (SL8) along with helper Arduinos. In both setups two cameras recorded mouse behavior and were positioned either below the mouse, to capture stimulus delivery and reflexive responses, or facing the test chamber, to capture the mouse's affective-motivational behaviors. In the first setup (FIG. 7A mice), the miniature microscope DAQ output a TTL that drove both cameras by triggering interrupt pins on an Arduino Uno, which then drove each camera, allowing us to synchronize each camera video frame with the Ca²⁺ imaging data. We used the Image Acquisition Toolbox in MATLAB (Mathworks) to collect TTL triggered video frames from each camera. A separate Arduino Mega element collected information on stimulus delivery times via a custom circuit that allowed the experimenter to select the current stimuli, using a keyboard (Adafruit, Product ID #1824) and LCD display (Adafruit, Product ID #772), and to click a button upon stimulus application to record the delivery time for later analysis. A third Arduino Uno measured the analog voltage signal from the accelerometer (Sparkfun ADXL335 or ADXL345) attached to the miniature microscope. The last Arduino measured the onset of licks and sent control signals to open a solenoid (NResearch, 161P011) to release sucrose. Both accelerometer and stimulus Arduino outputs had internal session times based off of each Arduino's internal clock as well as from the miniature microscope frame number (via the TTL) included in their output; we used the miniature microscope frame numbers to do the final synchronization in later analyses with the miniature microscope C²⁺ imaging data. Each data-collecting Arduino received a synchronizing TTL signal from the miniature microscope's DAQ and streamed data to a PC where we saved the information using a custom MATLAB script.

In the second setup (FIG. 7C mice), the first SL8 measured analog outputs from the miniature microscope attached accelerometer (100 Hz sample rate, see FIG. 1A), connected via a four-(Daburn Electronics & Cable, #2714/4) or a five-(Daburn Electronics & Cable, #2714/5 or Cooner Wire Company NMUF5/36-2550SJ) conductor wire; simultaneously, we recorded in the same SL8 timestamps for the onsets of licks and control signals for sucrose delivery (triggered on the rise of each signal pulse to obtain exact timing information). To collect the latter data, we designed a separate circuit consisting of two Arduinos (Uno and Mega) and a custom lick detector that measured the mouse's licks (signal #1) and sent a control signal (signal #2) to turn on a solenoid. The first SL8 recorded both signals #1 and #2. The second SL8 collected stimulus-onset times from a custom-designed circuit that allowed experimenters to select a stimulus and press a button to timestamp when they delivered a particular stimulus. Saleae software (Logic 1.2.xx) recorded and saved all data from each SL8. We wrote custom Python and MATLAB scripts to extract the data for use in subsequent analysis.

For all mice, we manually checked each session's annotated stimulus-onset time, using a custom MATLAB program to scroll manually through a video recorded from a camera positioned below the mouse. Using this program, we corrected instances in which the annotation did not match the actual onset time of stimulus delivery. To ensure accuracy, we frame-locked this camera to the miniature microscope by triggering each video frame collected by the microscope's DAQ ‘sync out’ TTL line. We used the stimulus timestamps collected in the imaging sessions of FIGS. 7A, 7C mice to automatically jump to the estimated stimulus onset time frame in the behavioral video, which facilitated the manual determinations of stimulus onset-times.

### Miniature Microscope Recording Parameters

We recorded all miniature fluorescent microscope videos at a frame rate of 10 or 20 Hz using between 213±3 and 390±7 μW LED light intensity (measured from miniature microscope GRIN with a Thorlabs PM100D and S120C) and saved each frame as a 12 bit image (of varying size, analyzed in a range of 250-275×250-270 pixels after down-sampling in each spatial dimension by a factor of 4 from the raw data). We used a stage micrometer (WARD's Natural Science, 94 W 9910) to empirically calculate each pixel to be 2.51 μm×2.51 μm.

### Noxious and Aversive Stimuli Experiments

We delivered a range of noxious, aversive, and appetitive stimuli to animals (FIG. 12): noxious cold (acetone), noxious heat (55° C. water), noxious pin (25G needle), air puff (300 ms), isopentylamine (˜85 mM in H₂O, delivered via 300-ms air puff), loud noise (˜85 dB for 300 ms, same as previously described), electric footshock (0.6 mA for 2 s), quinine (0.06 mM), and 10% sucrose. We habituated mice to a fear conditioning test chamber, similar to our previous setup (19), for 30 min on four consecutive days prior to conducting experiments. After mounting of the miniature microscope, we allowed mice to habituate for 10 min to the test chamber, followed by an additional 10 min of adlibitum access to 10% sucrose. We then followed the test procedure outlined in FIG. 12A and used the same data collection hardware as in FIG. 7C. Because the main behavior chamber and the fear conditioning chambers were in separate rooms, we allotted time for the mouse to rehabituate to the fear conditioning chamber for 10 min after the adlibitum quinine access. We cleaned all chambers with 70% ethanol before each experimental procedure.

We delivered noxious cold (acetone), noxious heat (55° C. water), noxious pin (25G needle), and loud noise (˜85 dB) as described above. Isopentylamine (Sigma-Aldrich SKU #126810, CAS #107-85-7) is an odor shown to be aversive in multiple previous studies (71-73). We placed 50 μL of isopentylamine onto a small piece of tissue paper (Kimtech, #05511) and placed this immediately into a 10-mL blood serum tube (Fisher #02685A) and re-capped. We then inserted two 16G needles through the tube cap and attached these to a valve (Gems Sensors and Controls, MB202-VB30-L203) controlling air delivery to a metal tube used to manually direct odorant to animals in the test chamber. We delivered air puffs through a blunt, 16G needle. We delivered both isopentylamine and air puff for 300 ms with medical-grade compressed air (UN1002) at between 20 to 30 PSI. We aimed isopentylamine and air puff stimuli during delivery at the nose and front half of the face, respectively. Mice received quinine (0.06 mM in deionized water (74)) after licking a metal tube in an identical manner as 10% sucrose but through a different tube to avoid contamination. For footshock trials, we habituated mice for 10 min followed by five deliveries of a 0.6-mA electric footshock, with 2 min between each stimulation. To synchronize the onset time of each footshock with Ca²⁺ imaging data and each behavior cameras' videos, we collected TTLs output by the miniature microscope DAQ and footshock software (Freeze Frame, Actimetrics) on a Saleae Logic 8 DAQ box, which allowed us to determine the specific image frames of the Ca²⁺ video that were synchronous with each footshock. We collected all subsequent data, processed the Ca²⁺ videos, and performed analyses as in the main protocol used in FIG. 7.

### Clozapine-N-Oxide Control Experiments

To check for possible alterations of neural activity in the presence of CNO alone (i.e., no hM4 expression), we conducted a shortened version of the main protocol (FIGS. 15D-15G). We injected mice with CNO (10 mg/kg), then placed them back in their home cage. After approximately 25 min, we mounted the miniature microscope on the mouse's head and placed them within the test chamber. They habituated for 10 min followed by adlibitum access to 10% sucrose solution delivered in the same manner as previously described. Mice then received a battery of stimuli: light touch (0.07-g von Frey filament), noxious pin (25G needle), or aloud noise (˜85 dB, same as previously described). We collected all subsequent data, processed Ca2+ imaging movies, and performed analysis similar to the main protocol used in FIG. 7.

### Processing Ca²⁺ Imaging Videos and Identifying Neurons

### Pre-Processing of Ca²⁺ Movies

We processed all Ca2+ imaging data in the MATLAB software environment using methods similar to previous studies (19, 20). To reduce computational processing times and boost signal-to-noise, we down-sampled imaging movies collected from the miniature microscope in both x and y lateral spatial dimensions using 4×4 bi-linear interpolation. To remove motion artifacts, we registered all frames in an imaging session to a chosen reference frame using Turboreg (75). Rather than register the entire frame, we selected and registered a sub-region of the field-of-view; this allowed us to choose a region with high-contrast features and without artifacts (e.g., dust particles on the optics) that could impede registration.

To improve the performance of motion correction, we first normalized the image frames by subtracting from each frame its mean value. We then spatially bandpass-filtered each frame of the movie (cutoff frequency: ˜0.10-0.16 cycle/μm using a Gaussian cut-off filter, which highlighted spatial features at the ˜6-10 μm scale). We performed an image complement operation on each frame, by subtracting each pixel value from the maximum pixel value in that frame (i.e., dark areas became light, and vice versa); this inverted the image and generally made the blood vessels and other dark static features appear more prominently, which benefited image registration. We obtained two-dimensional spatial translation coordinates from Turboreg by having the algorithm compare each processed frame to a reference frame (the 100th movie frame). We then used the translation values so obtained for each image frame to register the raw Ca²′ movie, but pre-processed in a different manner so as to aid cell extraction, rather than spatial registration.

To facilitate cell extraction, we divided each frame of the raw Ca²⁺ movie by a low-frequency bandpass-filtered version of itself (cutoff frequency: ˜0.0014-0.0063 or ˜0.0014-0.01 cycle/μm using a Gaussian cut-off filter). This served to diminish neuropil and other background fluctuations. We then registered the resulting image frames using the two-dimensional spatial translation coordinates obtained previously.

Since motion correction can cause the movie edges to take on inconsistent borders due to variable translations, we determined the maximum amount all frames were translated during the motion correction procedure in each dimension (tₘₐₓ) and then added a border of size tmax pixels extending from the edge of each frame toward the middle of the frame. We set a maximum border size (tₘₐₓ) of 14 pixels (˜35 μm). We converted each movie frame to relative changes in fluorescence using the following formula:

$$\frac{Δ F ( t )}{{F}_{0}} = \frac{F ( t ) - {F}_{0}}{{F}_{0}}$$

where F₀ was the mean image over the entire movie. Lastly, we temporally smoothed each movie by down-sampling from the original 20 or 10 Hz to 5 Hz; specifically, for a x×y×t movie, we bilinearly down-sampled in x×t to reduce computational processing times, which is equivalent to performing a 1D linear interpolation in time of the intensity values at each pixel. Extraction of neuron shapes, locations, and activity traces

After processing each session's Ca²′ imaging videos, we computationally extracted individual neurons and their activity traces using the PCA-ICA algorithm (76). We used the following parameters for PCA-ICA: p=0.1 and a maximum of 750 iterations. The parameter p is the relative weight of temporal information in ICA, and p=0.1 indicates we performed a spatio-temporal ICA with greater weight given to the spatial than to the temporal skewness. The algorithm output a series of candidate spatial filters (x×y×n) and temporal traces (n×f)—where n is the number of neurons, t is the frame, and (x, y) are spatial dimensions-associated with temporally varying sources, which we then manually verified as neurons.

### Manual Neuron Identification

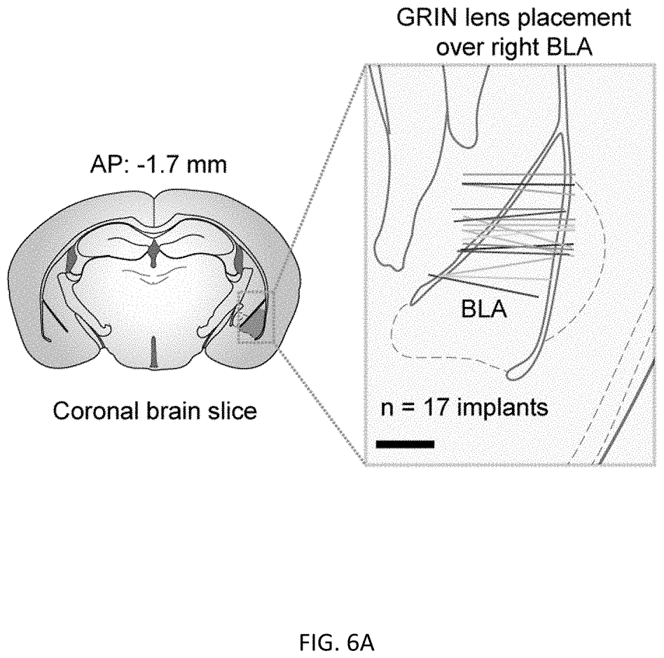

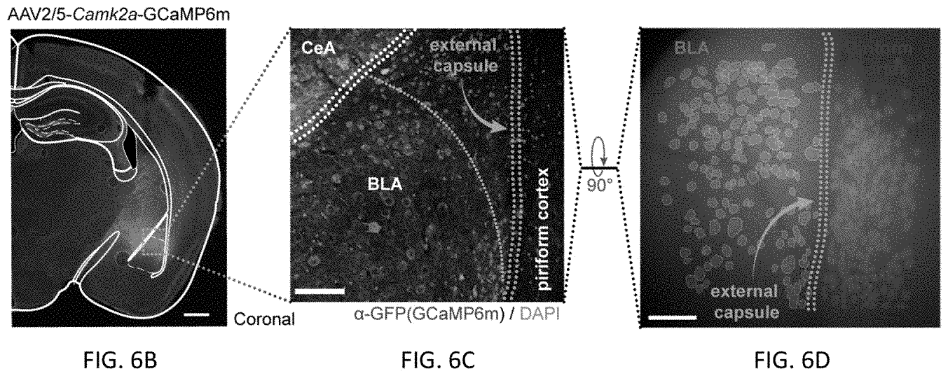

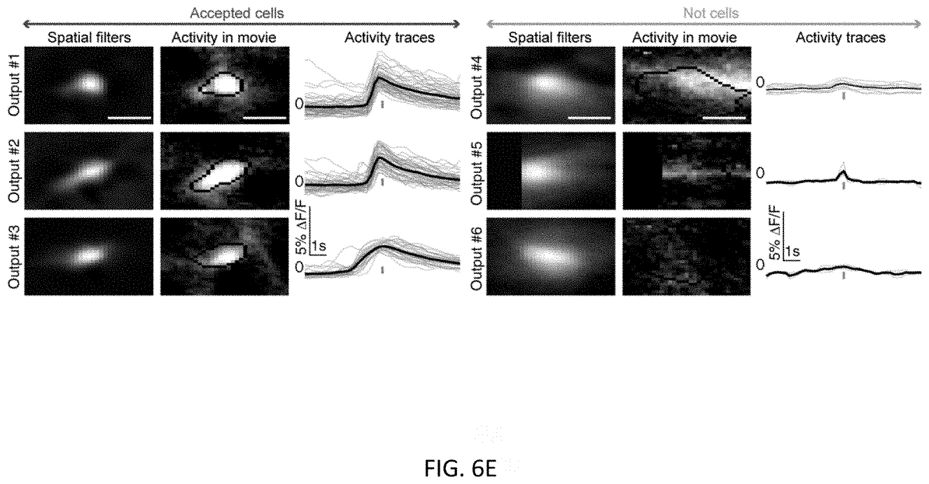

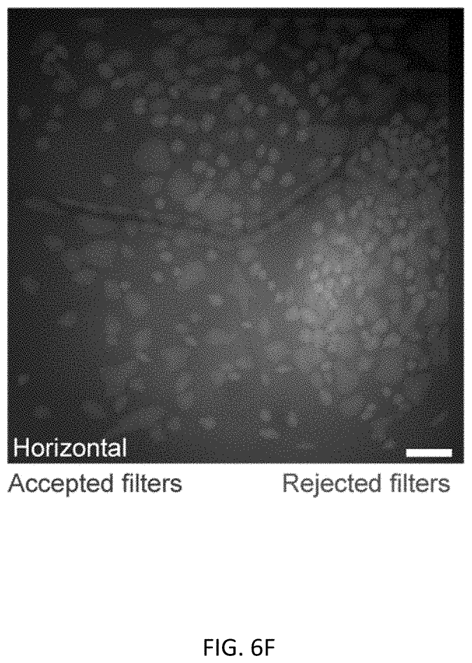

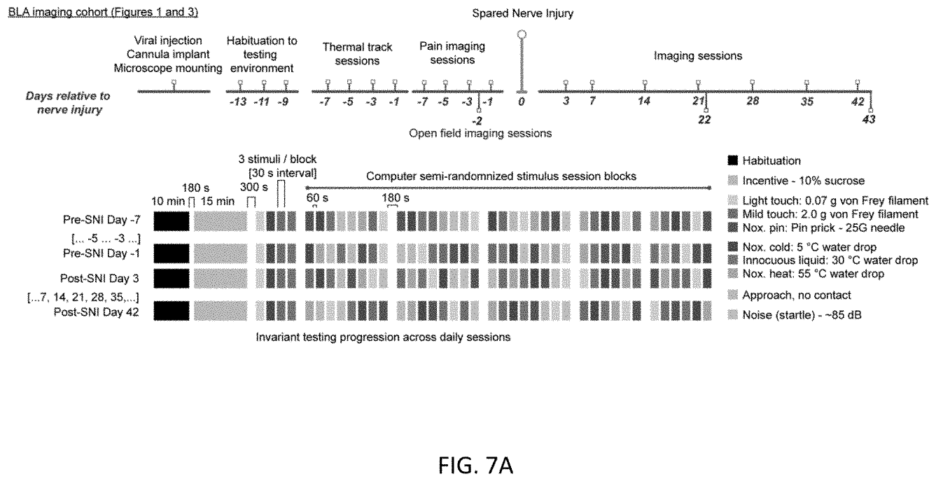

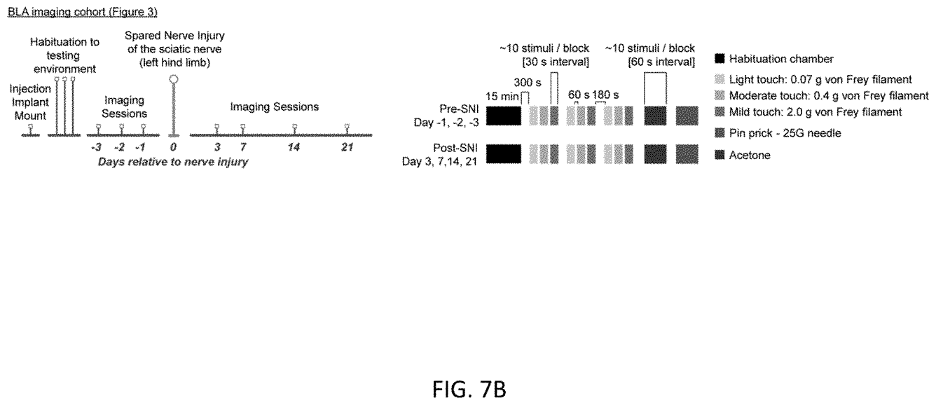

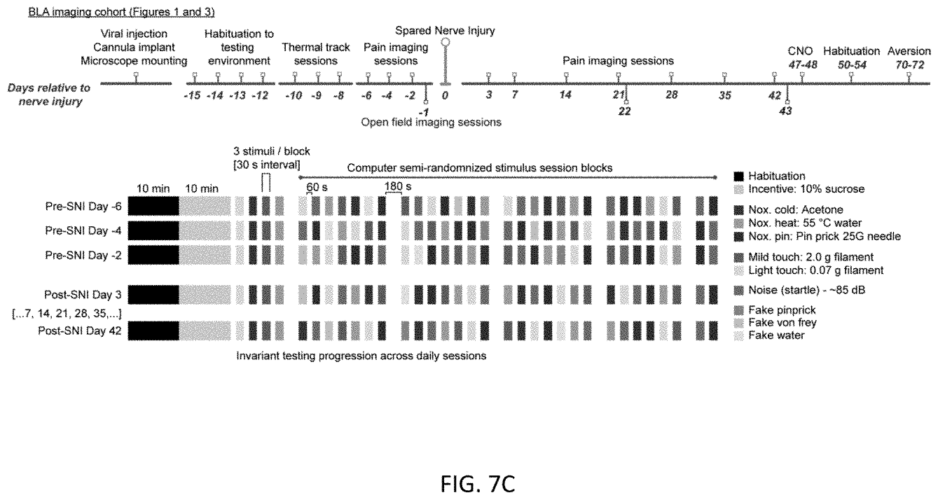

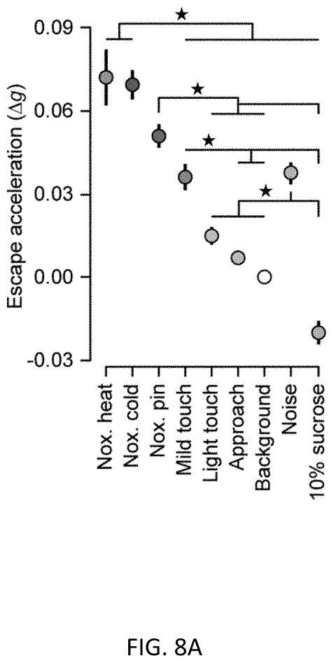

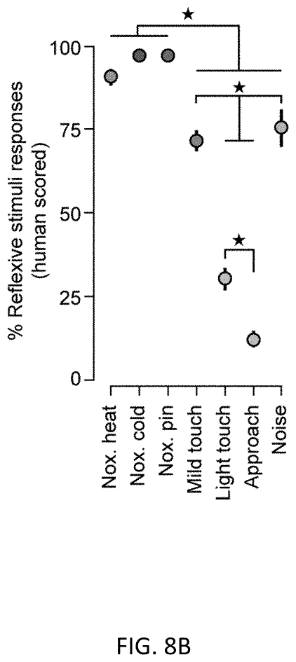

For all imaging sessions analyzed in this study, we used neurons manually identified by a single human scorer. For each imaging session, we loaded a custom MATLAB GUI that displayed the spatial filter and activity trace of each candidate cell, along with the candidate cell's average Ca²⁺ transient waveform. The human scorer also viewed a maximum projection image of all output spatial filters (FIG. 6F), on which the currently selected candidate cell was highlighted.

In addition, we noted that ICA (and other neuron extraction algorithms) often yielded candidate sources with images and activity traces that look highly similar to those of real neurons but that are actually associated with neuropil or other sources of contamination in the movie. Thus, we added another GUI interface to avoid including these false positives. Specifically, we cropped the movie to a 31 pixel×31 pixel (˜78 μm×−78 μm) region centered on the centroid of each candidate cell; we then created movies containing 10 frames before and after the onset of an individual peak in the candidate Ca²⁺ activity trace to help visualize actual transient-related activity in the movie. Each ICA output had up to 24 of these movies created based on each output's highest signal-to-noise (SNR) peaks. We spatially concatenated all of these movies associated with a specific ICA output to create a montage movie that allowed the human scorer to view movie data associated with peaks in the activity trace for each output at once, which eased decision-making. We used several criteria to classify an ICA output as a neuron: minimal overlap of an output's spatial filter with blood vessels or other contaminating signal sources, resemblance of each output's spatial filter to a 2D Gaussian or an expected neuron shape based on prior knowledge (FIG. 6E, “spatial filters”), similarity of the spatial filter to activity within the movie and proximity of output's centroid to movie activity (FIG. 6E, “activity in movie”), and similarity of the average transient waveform to a typical Ca²⁺ transient waveform as observed using GCaMP6, such as a fast rise time followed by a slow decay (FIG. 6E, “activity traces”). Using these criteria, on average a human scorer manually determined 39±1.4% (n=138 imaging sessions) of ICA outputs to be neurons (FIG. 6F).

For animals in which the internal capsule was present and neurons from the piriform cortex were within the imaging plane, we used a custom MATLAB GUI to manually select a region corresponding to the location of putative BLA neurons and excluded all other neurons in the imaging plane not within this region (FIG. 6D). We used an additional criterion regarding the cellular activity rate, since the piriform cortex often had higher overall rates of activity than BLA neurons that made them distinguishable. All references to ‘neurons’ within the context of miniature microscope imaging in this study refers to these manually curated BLA neurons from PCA-ICA and subsets therein (FIGS. 6D-6F).

### Ca²⁺ Transient Detection and Activity Trace Binarization

To detect Ca²⁺ events (used in analysis of movement-related, stimulus-induced, and spontaneous neuronal activity), we used a threshold-crossing algorithm similar to previously described methods (20). To reduce detection of spurious, high SNR noise, we spatially smoothed the signal by averaging over a 600 ms sliding window. To remove baseline fluctuations, we calculated a sliding median (40 s window) and subtracted this from the activity trace. To capture transient events during the rise time, we took the time-derivative of the resulting trace, calculated the standard deviation (a) for each signal, and identified any peaks that were ≥2.5 s.d. above baseline noise while enforcing a limit of a minimum inter-event time of >10 frames (2 s). We created a binarized activity vector for each neuron in which all frames associated with candidate peaks were assigned values of one and all other non-event frames assigned values of zero. We concatenated all n neurons binarized activity vectors into an n×f matrix that we used in subsequent analysis, where indicated.

### Calculation of Spontaneous Firing Rate

To assess whether the spontaneous firing rate of BLA neurons changed. For all mice run through the FIG. 7 protocols and with available baseline miniature microscope imaging session data (either 10 or 15 min), we calculated the mean event rate irrespective of the animal's movement, or other states, during the baseline period. For all mice, we defined Ca²⁺ transients as in “Ca²⁺ transient detection and activity trace binarization” and determined the mean Ca²⁺ event rate by calculating the mean rate of Ca²⁺ transients during the baseline period for each neuron. To calculate the mean Ca²⁺ event rate across the neuron population, we took the mean over all neurons' spontaneous rates. To compare spontaneous rates across mice, and before and after the mice were in a neuropathic state, we first calculated the mean population firing rate of all pre-SNI or—sham surgery sessions for each mouse individually. We then used this mean value to normalize the spontaneous Ca²⁺ population event rates measured for the same animal during all subsequent imaging sessions (FIGS. 19A-19B). We calculated the nociceptive ensemble firing rate (FIGS. 14A-14B) identically, but the final population rate only included neurons within that session that were classified as within the nociceptive ensemble (see Definition and calculation of nociceptive ensemble).

Identification of Neurons with Significant Stimulus-Evoked Responses

### Identification of Responsive Neurons

To determine which neurons significantly responded to a given stimuli, we took neuronal activity data (PCA-ICA output traces) from a 2-s-post-stimulus interval for all trials (creating a n×t×f matrix, where n=number of neurons, t=number of trials, and f=number of frames per trial) and binned it into 1-s bins by taking the mean of each bin's ΔF/F activity. For each cell, we then compared the binned activity response values to those in an identically binned 2-s window from −5 s to −3 s before the stimuli. We pooled this activity across all presentations of a specific stimulus and calculated a p-value for each neuron using a one-tailed Wilcoxon rank-sum. We designated any neurons for which P<0.01 as being significantly responsive to a given stimulus.

### Definition and Calculation of Nociceptive Ensemble

We defined the BLA nociceptive ensemble in two ways throughout this study. For studies of mice in a normal, non-neuropathic state (FIG. 1), we defined the nociceptive ensemble as consisting of neurons responsive to noxious pin (25G needle), noxious heat (55° C. water), or noxious cold (5° C. water). When mice were in a neuropathic state or had undergone a sham surgery (FIG. 3), we defined the nociceptive ensemble as neurons responsive to either noxious pin or noxious cold (5° C. water or acetone) (FIGS. 7A, 7C animals) or to either noxious pin or acetone (FIG. 7B animals). In all cases, we separately assigned stimuli responsive neurons to the nociceptive ensemble for each session using the above definitions. First, we identified significantly responding neurons (see Calculation of stimuli responsive neurons) for each stimulus individually, and then we identified neurons responding to any stimuli within the above definitions as part of that session's nociceptive ensemble. For specific cases as noted within the text, we restricted subsequent analyses to neurons within the nociceptive ensemble.

### Spatial Distributions of Significantly Responsive Neurons

To calculate the spatial distributions of significantly responsive neurons, we first computed each neuron's centroid location. For each neuron's x×y spatial filter output by PCA-ICA, we binarized the image by calculating the maximum value and set all values below 50% of this value to ‘zero’ (not part of the neuron) and the remainder to ‘one’ (part of the neuron). We then set to ‘zero’ any pixels not connected to the maximum value using a union-finding algorithm implemented in a standard MATLAB function. The x and y coordinates for all parts of each neuron's spatial filter image that are still labeled ‘one’ were found and multiplied by their true values in the original spatial filter imaged. We then calculated the arithmetic mean of each dimension's weighted coordinate vector and rounded it to the nearest whole pixel value. This allowed us to obtain centroids that are centered closer to the peak intensity of the spatial filter. We converted all neuron centroid pixel values to metric units (2.51 μm/pixel) and computed the full pairwise Euclidean distance matrix for all neuron-neuron pairs in a session. We then binned distances in 1-μm increments and the empirical cumulative distribution calculated for both all neurons and only for neurons significantly responsive to each stimulus (FIG. 9H).

### Cross-Day Analysis of BLA Neuronal Activity

To match neurons across days we implemented a multi-step algorithm similar to previously published work (19, 20). We thresholded spatial filters from PCA-ICA by setting to zero any values below 40% the maximum for each spatial filter and used these thresholded filters to calculate each neuron's centroid, see “Spatial distribution of significantly responsive neurons and neuron centroid calculation”. We modified that procedure for cross-day alignment by not rounding each neuron's centroid coordinates to the nearest pixel value in order to improve accuracy of cross-day alignment. We created simplified spatial filters that contained a 10-pixel-radius circle centered on each neuron's centroid location; this allowed us to register different days while ignoring any slight day-to-day differences in PCA-ICA's estimate of each neuron's shape even if the centroid locations were similar.

For each animal, if we had N sessions to align, we chose the N/2 session (rounded down to the nearest whole number) to align to (align session) in order to compensate for any drift that may have occurred during the course of the imaging protocol. For all other imaging sessions, we first created two neuron maps based on the threshokied spatial (“thresholded neuron maps”) and 10-pixel-radius circle (“circle neuron maps”) filters described above (see FIG. 10A) by taking a maximum projection across all x and y pixels and spatial filters (max in 3¹ dimension of x×y×n neuron filter matrix, where n=neuron number). We registered these neuron maps to the align session using Turboreg (75) with rotation enabled for all animals and isometric scaling enabled for a subset of animals in cases where that improved results. First, we registered the threshokied neuron maps for a given session to the align session. Second, we used the output 2D spatial transformation coordinates to also register the circle neuron maps followed by registration of the circle neuron map with that animal's align session. We applied the resulting 2D spatial transformation coordinates to the thresholded neuron map. We repeated this procedure at least five times (FIG. 10A). We used the final registration coordinates to transform all spatial filters from that session so they matched the align session's spatial filters and repeated this process for all sessions for each animal individually.

After registering all sessions to the align session, we then recalculated all the centroid locations as described above. We set the align session centroids as the initial seed for all global cells. Global cells are a tag to identify which neurons are matched across imaging sessions; for example, global cell #1 might be associated with neurons #1, #22, #300, #42, and #240 across each of five imaging sessions, respectively. Starting with the first imaging session for an animal, we calculated the pairwise Euclidean distance between all global cells' and the selected session's neurons' centroids. We then determined any cases in which a global cell was within 5 μm (nominally ˜2 pixels) of a selected session's neurons. In such cases, the algorithm added that neuron to that global cell's pool of neurons, the global cell's centroid recalculated as the mean location between all associated session neurons' centroid locations, and any unmatched neurons in that session annotated as new candidate global cells. We repeated this process for all sessions associated with a given animal (see FIGS. 10A-10C).

After assigning all neurons across all animal's imaging sessions to a global cell, we then conducted a manual visual inspection of each animal's cross-day registration. We removed imaging sessions that did not align well with other sessions associated with a particular animal. This led to us removing n=42 sessions from this analysis across all FIG. 7 mice. In addition, to quantify our alignment accuracy, we calculated the pairwise distance between all session neurons' centroid locations that are associated with a common global cell and showed that the majority of alignment was below 5 μm (FIGS. 10D-10E). We further confirmed this by taking all global cells associated with at least two or more neurons and comparing their associated neurons' centroid location with the global cell's centroid location (FIG. 10F).

To calculate the number of sessions a global cell responded to specific stimuli, we used the classification of significantly coding neurons in “Determination of significantly responding stimuli neurons”. We then checked for each global cell the number of sessions it responded to a given stimuli while ignoring any global cells who only had activity on a single session (FIGS. 10G-10I). To calculate maximum duration of stimulus responsivity, and because not all sessions were run exactly on the specified protocol days, we used the actual date the imaging session took place on to calculate both the earliest and latest date that a global cell significantly responded to each stimulus and took the difference to obtain a measure for how long a neuron stably coded for said stimulus (FIG. 10J).

### Analysis of the Overlap in Neural Ensembles Responsive to Different Stimuli

We sought to determine whether the neuronal ensembles responsive to two different stimuli were consistent with a hypothesis of statistical independent coding channels. To test this hypothesis, we needed to compute the likelihood that statistically independent assignments of cells' coding identities would yield the observed level of overlap in the two coding ensembles. There are two ways to calculate the expected level of overlap under an assumption of independence. Prior methods used bootstrapping to estimate an empirical null distribution and compared the actual overlap to that. Here we introduce an alternative, exact solution.

We calculated the extent to which the observed overlap was unexpected by chance as a specific instance of the classic statistics thought-experiment of drawing without replacement balls from an urn containing black and white balls. In our case, we had a population of N neurons and were seeking the probability, p, of having k successes (number of significant neurons for stimulus #2) in a population with pre-defined Ksuccesses (number of significant neurons for stimulus #1) in n drawings (number of significant neurons for stimulus #2). Using the hygecdf and hygestat functions in MATLAB, we calculated p and the expected number of overlap neurons given the actual number of significantly responsive neurons observed for stimuli #1 and #2 (FIG. 13C). We validated the results through comparisons to shuffle tests based on the same parameters and using 1,000 rounds of 1,000,000 shuffles to construct bootstrapped distributions (FIG. 13D). Because the two methods attained nearly identical results, we used the hypergeometric distribution instead of shuffle tests to reduce computational processing times and to obtain an exact p-value.

To determine whether the overlap in coding ensembles became more expected than chance, either before or after spared nerve injury (FIG. 19E), we performed Wilcoxon rank-sum tests in the R programming language using a Benjamini-Hochberg multiple comparisons correction (77) to identify whether the overlap differed significantly that expected by chance (see FIG. 13E).

### Statistical Analyses

We performed all statistical analyses within the R or MATLAB (2015b or 2017a) software environments, unless otherwise noted. Throughout the text, “signed-rank” and “rank-sum” tests refer to Wilcoxon signed-rank and rank-sum tests, respectively. We used the Benjamini-Hochberg (B-H) procedure for all non-ANOVA multiple comparisons correction (77). For ANOVA analyses, we performed either a one-way or two-way repeated measures ANOVA via the aov function in R followed by a Tukey test, when appropriate. When comparing specific hypotheses, we ran the necessary pairwise statistical test followed by a B-H correction. We did not blind the experimenters performing the imaging analyses regarding the cohorts (neuropathic or uninjured) or pain states (pre- or post-SNI) of the mice. However, we used identical code and analysis methods for all cohorts throughout the study. Unless otherwise noted, values and error bars in the text denote means±SEM.

### Code and Data Availability

For Ca²⁺ imaging video motion correction, the C code is available on the author's website (75). Our MATLAB implementation of the image registration is also available upon request. Code used for pre-processing Ca²⁺ imaging data, neuron identification and activity trace extraction, ICA output manual cell classification GUI, and animal behavior tracking is available at (46). Any other code used in this study's findings, to generate graphs and perform statistical analysis, are available upon reasonable request.

The datasets of this study, approximately 43 TB in size, are available upon reasonable request to the corresponding authors.

### Histology for Miniature Microscope Mice Tissue

We transcardially perfused all mice used in the imaging protocol in FIGS. 7A, 7C with 4% formalin in PBS (Fisher Scientific, NC0238527). We stored brains in 4% formalin in PBS and sectioned them at 100 μm using a vibratome (Leica VT1000S). For staining tissue sections, we washed sections three times in PBS with 0.3% Triton-X100 for 5 min each, blocked with 10% Donkey Serum (Jackson Immunoresearch, 017-000-121 Normal Donkey Serum) in 0.3% Triton-X100 in PBS for 1 hr at room temperature, and stained with primary antibody (Invitrogen α-GFP A11122 rabbit at 1:1000 dilution) overnight at 4° C. The following day (all procedures at room temperature), we washed sections three times for 5 min each in 0.3% Triton-X100 in PBS, stained with secondary antibody (DyLight 549 Donkey α-rabbit at 1:500) for 90 min, stained with DNA stain (AppliChem DAPI BioChemica, 50 nm/mL in 1×PBS) for 20 min, and performed a final wash in 1×PBS. We mounted slices onto glass coverslips with mounting media (SouthernBiotech, Fluoromount-G cat no. 0100-01). We acquired large field-of-view images (FIG. 6B) with a standard fluorescence macroscope (Z16, Leica) while we collected zoomed in images (FIG. 6C) with a two-photon (Prairie Technologies, Ultima Multiphoton Microscopy System using Olympus LUCPLFLN 20× objective). Where applicable, we only adjusted raw miniature microscope histology images with linear manipulations of contrast and brightness.

### Chronic Neuropathic Pain Model

To induce a chronic pain state, we used a modified version of the Spared Nerve Injury (SNI) model of neuropathic pain, as previously described (70). This model entails surgical section of two of the sciatic nerve branches (common peroneal and tibial branches) while sparing the third (sural branch). Following SNI, the receptive field of the lateral aspect of the hindpaw skin (innervated by the sural nerve) displays hypersensitivity to tactile and cool stimuli, eliciting pathological reflexive and affective-motivational behaviors (allodynia). To perform this peripheral nerve injury procedure, anesthesia was induced and maintained throughout surgery with isoflurane (4% induction, 1.5% maintenance in oxygen). The left hind leg was shaved and wiped clean with alcohol and betadine. We made a 1-cm incision in the skin of the mid-dorsal thigh, approximately where the sciatic nerve trifurcates. The biceps femoris and semimembranosus muscles were gently separated from one another with blunt scissors, thereby creating a <1-cm opening between the muscle groups to expose the common peroneal, tibial, and sural branches of the sciatic nerve. Next, −2 mm of both the common peroneal and tibial nerves were transected and removed, without suturing and with care not to distend the sural nerve. The leg muscles are left uncultured and the skin was closed with tissue adhesive (3M Vetbond), followed by a Betadine application. During recovery from surgery, mice were placed under a heat lamp until awake and achieved normal balanced movement. Mice were then returned to their home cage and closely monitored over the following three days for well-being.

### Targeted Recombination in Active Populations (TRAP) of BLA Neural Ensembles

For all TRAP Procedures, Stereotaxic Bilateral Injections of Viral Reagents Occurred 3-5 Weeks Prior to TRAP. Please See FIGS. 14D and 20A for Schematic Experimental Timelines.

### Acute Nociceptive TRAP (Noci-TRAP)

We habituated mice to a first testing room (room-A) for three consecutive days. Execution of all TRAP procedures occurred in Room-A. During these habituation days, no nociceptive stimuli were delivered, and no baseline thresholds were measured (i.e., mice were naïve to pain experience before the TRAP procedure). In room-A, we placed individual mice within red plastic cylinders (10.16-cm D), with a red lid, on a raised perforated, flat metal platform (60.96-cm H). The male experimenter's lab coat was present in the testing room for the first 30 min of acclimation, and then the experimenter entered the room for the final 30 min of habituation; this was done to mitigate potential alterations to the animal's stress and endogenous antinociception levels. To execute the TRAP procedure, we placed mice in their habituated cylinder for 60 min, and then a 25G sharp pin was applied to the central-lateral plantar pad of the left hindpaw (tibial-sural nerve paw innervation territory), once every 30 s over 10 min. This stimulus frequency was selected to closely match the protocols used in the microendoscope imaging experiments in which significant Ca² transients were reliably detected in BLA Camk2a+ neurons. Following the pin stimulations, the mice remained in the cylinder for an additional 60 min before injection of 4-hydroxytamoxifen (20 mg/kg in ˜0.25-mL vehicle; subcutaneous). After the injection, the mice remained in the cylinder for an additional 2 hrs to match the temporal profile for c-FOS expression, at which time the mice were returned to the home cage (Note: an immediate return to the home cage following the pin stimulations was considered, but ultimately avoided as potential safety-related neural activity could occur and thus TRAP BLA neurons of putative positive valence in addition to the nociceptive ensemble). To mitigate the influence of contextual memory recall from the noxious TRAP procedure, all subsequent behavioral assays occurred in a second testing room (room-B). In room-B, we placed the noci-TRAP mice within different holding chambers (7.62×15.25×15.25 cm plastic chamber [white opaque walls]), atop a different metal platform floor (smooth hexagon-hole perforated sheet, McMaster-Carr, #92725T22). Furthermore, the experimenter wore daily disposable lab coats; different from the coat used in the room-A context. After completion of all experiments, we perfused mice and dissected the brains for verification of hM4-mCherry expression in the BLA. We excluded mice with off-target viral expression in the central amygdalar nucleus from the behavioral analysis. Based on this criteria, n=7 mice study were removed from the final analysis.

### Chronic Neuropathic Pain TRAP (Neuropathic-TRAP)

We habituated mice inside individual red plastic cylinders (10.16-cm D) on a raised flat, perforated metal platform (60.96-cm H) for 3 days prior to the start of behavioral sensory testing. After basal thermal and mechanical thresholds were measured, mice underwent a peripheral nerve injury surgery (Spared Nerve Injury, SNI; see “Chronic neuropathic pain model” above for details of the surgical procedure). At Day 21 post injury, when mice display significant mechanical and thermal hypersensitivity at the plantar surface of the left hindpaw, we habituated mice as stated above (see “Acute nociceptive TRAP (noci-TRAP)). To execute the light touch-TRAP procedure, a von Frey filament (0.07-g) was lightly applied to the lateral aspect of ventral hindpaw (sural nerve innervation receptive field) with enough force to cause a slight bend of the filament for up to 1 s before being retracted. The filament stimulus was applied once every 30 s over 10 min. We selected this stimulus frequency to closely match the protocols used in microendoscope imaging experiments. Following the filament stimulations, the mice remained in the cylinder for an additional 60 min before injection of 4-hydroxytamoxifen (20 mg/kg in ˜0.25-mL vehicle; subcutaneous). After the injection, the mice remained in the cylinder for an additional 2 hrs, at which time the mice were returned to the home cage. At Day 28 post injury, we confirmed neuropathic hypersensitivity persisted. Subsequent behavioral studies to assess chronic neuropathic hypersensitivity and affective-motivational behaviors were conducted beginning at Day 42 post SNI in order to allow sufficient expression of the viral DREADD cargo. After completion of all experiments, we perfused mice and dissected the brains for verification of hM4-mCherry expression in the BLA. We excluded mice with off-target viral expression in the central amygdalar nucleus from the behavioral analysis. Based on this criteria, n=5 mice were removed from the final analysis.

### Optogenetic Nociception TRAP (o-T RAP)

Different AAV serotypes display unique infection tropisms. In particular, serotype-6 shows a preferential infection of peripheral primary afferent nociceptor populations (80). To express the light-sensitive cation channel channelrhodopsin2 (ChR2) in putative primary afferent nociceptors, we intrathecally injected AAV6-hSyn-ChR2(H134R)-eYFP immediately following the i.c. BLA injections of AAV-DIO-DREADD(Gi)-mCherry in TRAP mice while remaining anesthetized under isoflurane (1-2% maintenance). Specifically, we shaved a small patch of fur on the back, wiped with alcohol and Betadine, and then inserted a 33G beveled needle connected to a WPI Nanofil syringe between the L5/L6 vertebrae and through the dura (confirmation by presence of reflexive tail flick). We slowly administered the virus was over 20 s. We retuned mice to their home cage for 4-6 weeks before behavioral verification of ChR2 expression. In pilot studies, we observed that intrathecal delivery of AAV6 does not uniformly infect all dorsal root ganglion (DRG) neurons across segmental levels. As we sought expression in lumbar DRGs for the purposes of our behavioral experiments that involve sensory testing on the hind limbs, we performed a behavioral screening of each mouse for transdermal light-responsivity when light was applied to the hindpaw. We placed mice inside individual red plastic cylinders (10.16-cm D) on a thin glass surface. A remotely movable fiber optic arm, connected to a 453-nm LED light source (SugarCube) below the glass (−′8 mm from the fiber tip to the plantar surface of the paw), was positioned under the heel of the left hindpaw, and a 453-nm −′1-s light pulse was delivered (3 mW/mm²). We measured whether an immediate nociceptive hindpaw reflex and/or pain affective-motivational behaviors (described below) occurred in response to the light indicated ChR2 expression in nociceptors. If no immediate responses were observed, the fiber optic was moved distally toward the toes and the stimulation was repeated; the location of light-responsivity on the paw was noted for future targeting during the TRAP protocol. We excluded mice from this experiment that exhibited no light-evoked pain behaviors. One week later, we habituated mice on the glass surface for 3 consecutive days (no blue light stimulus was given). Next, on the day of the TRAP procedure, we placed mice inside the cylinders for 30 min. The fiber optic was positioned under the left hindpaw at the previously noted light-responsive site, and we then delivered transdermal light pulses (1 s, 3 mW/mm²) once every 30 s over 10 min. Following light stimulations, mice remained in the cylinder for an additional 60 min before injection of 4-hydroxytamoxifen (20 mg/kg in −′0.25-mL vehicle; subcutaneous). After the injection, mice remained in the cylinder for an additional 2 hrs, at which time we returned mice to their home cage. Subsequent behavioral experiments were performed 5-8 weeks later.

### Behavioral Quantification of Acute and Chronic Pain Behaviors

For all Behavioral Tests the Experimenter was Blind to Either the SNI Vs. Sham Procedure, or the Injection of CNO Vs. Saline. Classification of Mouse Pain Behaviors into Reflex and Affective-Motivational Behaviors

In mice, we previously reported our observation that a cutaneous noxious stimulus can elicit several distinct behavioral responses (81, 82): 1. Withdrawal reflexes: rapid reflexive retraction or digit splaying of the paw that occur in response to noxious stimuli, but cease once the noxious stimulus is removed; and 2. Affective-motivational behaviors: temporally-delayed (relative to the noxious stimulation), directed licking and biting of the paw (termed “attending”), extended lifting or guarding of the paw, and/or escape responses characterized by hyperlocomotion, rearing or jumping away from the noxious stimulus. Please see FIG. 15A for an illustrative example of these nociceptive reflex and affective-motivational behaviors. Paw withdrawal reflexes are classically measured in studies of sensitivity and involve spinal cord and brainstem circuits (as these behaviors are observed in decerebrated rodents (83)). In contrast, affective-motivational responses are complex behaviors requiring processing of nociceptive information by brain limbic and cortical circuits. The presence of these complex behaviors indicates the subject's motivation to make the aversive sensation cease, by licking the affected tissue, protecting the tissue, or seeking an escape route (83-92).

### Pain Affective-Motivational and Nociceptive Reflex Behavioral Assays

To evaluate mechanical reflexive sensitivity, we used a logarithmically increasing set of 8 von Frey filaments (Stoelting), ranging in gram force from 0.07- to 6.0-g (93). These filaments were applied perpendicular to the plantar hindpaw with sufficient force to cause a slight bending of the filament. A positive response was characterized as a rapid withdrawal of the paw away from the stimulus within 4 s. Using the Up-Down statistical method, the 50% withdrawal mechanical threshold scores were calculated for each mouse and then averaged across the experimental groups. The response frequency was calculated as the number of positive responses out of 10 stimulations, delivered at 30-s intervals.

To evaluate affective-motivational responses evoked by mechanical stimulation, we used three von Frey filaments (0.07-g, 0.4-g, and 2.0-g) and a sharp 25G syringe needle (pin prick) (94). Each filament was applied for 1 s and the pin prick was applied as a sub-second poke to the hindpaw, and the duration of attending behavior was collected for up to 30 s after the stimulation. Only one stimulation per filament was applied on a given testing session.

To evaluate affective-motivational responses evoked by thermal stimulation (81), we applied either a single, unilateral 50-μL drop of water (5, 30, or 55° C.) or acetone (evaporative cooling) to the left hindpaw, and the duration of attending behavior was collected for up to 60 s after the stimulation. Only one drop stimulation was applied on a given testing session. To evaluate adaptive thermal avoidance and temperature preference, we placed mice in the center of a linear Thermal Gradient Track (121.92-cm L×8.25-cm W metal alloy floor; 15.24-cm H black plastic walls), with the floor featuring a temperature gradient along the long axis. Mice freely explored the track for 60 min. To create the temperature gradient, we placed either heating or cooling plates (Bioseb) under the outermost 16.51 cm of the metal floor, with one plate set to 50.0° C. and the other set to 0.0° C., creating an actual floor gradient of 48° C.→5° C., respectively. The track was subdivided into 25 temperature zones (4.8-cm D per zone), and we assessed the temperature at the center of each zone by a K-probe thermocouple. “Noxious zone blocks” were designated based on the temperature thresholds for nociceptive behaviors (>42° C. and <17° C.). The track was illuminated by a centered, overhead light (104 lux), and the ambient room temperature was 26° C. Only one mouse was present in the room during all trials. A video camera placed above the track recorded the position of the mouse within the temperature zones, and videos were later analyzed using a video-tracking software (Etho-vision, Noldus) for duration of zone occupancy, zone visits, distance, velocity, and acceleration.

To evaluate active avoidance and escape behaviors to optogenetically driven nociception, mice expressing ChR2 in peripheral primary afferent nociceptors freely explored a custom-built two-choice chamber with LED-lit floor panels. A 32×32 LED array (8×8 cm) illuminated half the array in blue light, and the other half in red light (˜0.3 mW/mm²). A thin glass surface (0.5 cm thick) covered the array floor, upon which we fitted a black plastic chamber (38 cm H) with a center divider wall containing a square passage hole (5-cm D) raised 2.5 cm from the array floor. The entire LED chamber was maintained in a quiet room with low ambient light (˜5 lux). We first placed mice in the red-light chamber and then allowed them to freely explore the chambers for 15 min. A camera placed above the chamber recorded the location of the mouse in the apparatus. We manually scored the videos to determine the time spent by the mouse in each chamber (automated tracking was not possible given the light from the LED floor). Only one mouse was present in the room during all trials.

To evaluate neuropathic adaptive cool/cold avoidance behavior, mice with SNI freely explored a two-temperature choice chamber. The chamber was constructed from adjoining two thermal plates (Bioseb): one reference plate set at 30° C., and a test plate with the temperature adjusted to either 30, 25, 20, 15, or 10° C. for independent trials. The test plate temperature order was randomized for each trial within the day. The chamber (white opaque plastic with no distinguishing features and no divider, 30.48×15.24×15.24 cm) was fitted onto the conjoined plates. At Day 56 post SNI and at 30-min post CNO injection, we placed mice on the reference plate facing the back wall. Mice then freely explored the chamber for 5 min, while an overhead camera recorded the chamber position and locomotion. After each trial, the mouse was returned to the holding cylinder, while the test plate temperature was rapidly cooled or heated to the next randomly assigned temperature trial. This procedure was repeated until all temperature trials were collected (6 temperature trials total). Video files for each trial were later analyzed using automated tracking software (Ethovision, Noldus) for path tracking, time spent on the test plate, and number of entries onto the test plate.

### Anxiety-Like Assays

The Elevated Plus Maze apparatus was made of blue plastic floors, and consisted of two open arms (30×8 cm), two arms enclosed in black plastic walls (30×8×30 cm) extending from a central platform (8×8×8 cm) at 90 degrees in the form of a “+”. The maze was elevated 30 cm above the floor. We placed individual mice in the center of the apparatus, as an overhead video camera recorded the locomotor paths throughout the 15 min trial. A diffuse overhead fluorescent light (102 lux) illuminated the track. The ambient room temperature was 26° C. Only one mouse was present in the room during all trials. Videos were later analyzed using a video-tracking software (Ethovision, Noldus) for distance, velocity, time spent in the open arms (body center-point tracking), and entries to the open arm (nose-point tracking).

The open field chamber (circular, 60.96-cm D, 38.1-cm H, opaque white polyethylene walls and floor) was divided into a central zone (center, 25-cm D) and an outer zone (peripheral). We placed individual mice in the peripheral zone, facing toward the chamber wall as an overhead video camera recorder the locomotor paths throughout the 15-min trial. A diffuse overhead fluorescent light (102 lux) illuminated the track, and the ambient room temperature was 26° C. Only one mouse was present in the room during all trials. Videos were later analyzed using a video-tracking software (Ethovision, Noldus) for total distance traveled, total time spent in the center zone, and mean locomotion velocity as the mouse exited the center zone.

### Sucrose-Water Preference Assay

To evaluate incentive motivational behavior, we placed mice in a custom-made plastic chamber (7.62×15.25×15.25 cm, 3 white opaque walls, 1 clear plastic wall for video monitoring) with two rounded gavage syringe spouts protruding from small holes in one of the two side walls: one spout dispensed room temperature water, while the other dispensed a room temperature 10% sucrose solution (in water). Mice then had 20 min to freely sample the spouts. A custom microprocessor-controlled release of either solution, which were set to dispense 12 μL of solution upon the first lick, with a minimum 1-s interval between all subsequent lick-induced dispensions. An Arduino using a custom circuit design described previously recorded the number of licks and lick rate while experimenters recorded consumption volume for each spout. To enhance the propensity of mice to actively sample the lick spouts, mice were water deprived 5-8 hr prior to the start of experiments. We repeated the protocol for 7 consecutive days to determine whether any changes in sucrose preference occurred.

### Histology for BLA, Dorsal Root Ganglion, and Spinal Cord Tissue

### Immunohistochemistry

Anesthetized mice (Fatal-PLUS, Vortech Pharmaceuticals) were transcardially perfused with room temperature 0.1 M phosphate buffered saline (PBS), followed by 10% formalin in 0.1 M PBS. The brain, DRG (L3-L5), and/or spinal cord (lumbar cord L3-L5 segments) were dissected, post-fixed overnight (brains) or for 4 hrs (DRG or cord) at 4° C., and cryoprotected in 30% sucrose in PBS. Tissues were then frozen in O.C.T. (Sakura Finetek, Inc.). Tissue sections (50 μm for brains; 30 μm for spinal cord; and 10 μm for DRG) were prepared using a cryostat (Leica Biosystems) and blocked with PBS containing 5% normal donkey serum and 0.3% Triton X-100 for 1 hr at room temperature. The sections were then incubated overnight with primary antibodies at 4° C. For the chicken anti-GFP antibody, the incubation was performed at 37° C. for 2 hrs. After extensive wash with PBS containing 1% normal donkey serum and 0.3% Triton X-100, sections were incubated with appropriate secondary antibody conjugated to AlexaFluor for 2 hrs at room temperature. Sections were then mounted in the glass slide with Fluoromount (Southern Biotech) after washing with PBS for 3 times for 5 min. Images were collected under a Leica TCS SP511 confocal microscope with LAS AF Lite software (Leica Microsystems).

The following primary antibodies were used: Anti-c-Fos (Rabbit, Abcam # ab7963-1), Anti-c-Fos (Rabbit, Synaptic Systems #226003), Anti-CGRP (Sheep, Abcam # ab22560), Anti-GFP (Chicken, Aves Labs # GFP-1020), Anti-RFP (Rabbit, Abcam # ab62341), Anti-NeuN (Mouse, Millipore # MAB377), Anti-Ret (Goat, R&D Systems # AF482), Anti-NF200 (Chicken, Aves Labs # NFH0211).

### In Situ Hybridization

Anesthetized mice (C57BI/6J, male, 5-8 weeks, Fatal-PLUS (Vortech Pharmaceuticals)) were transcardially perfused with 0.1 M PBS followed by 10% formalin in 0.1 M PB. Brains were dissected, cryoprotected in 30% sucrose overnight, and then frozen in OCT. Frozen tissue was cut into 14 μm thick slices, placed onto Superfrost Plus slides, and kept at −80° C. Tissue was thawed from −80° C., washed with PBS at room temperature, and subsequently processed according to the Advanced Cell Diagnostics RNAscope Technology protocol (ACD Bioscience). We first washed the tissue with solutions from the pretreatment kit to permeabilize the tissue, incubated with protease for 30 min followed by the hybridization probe(s) for 2 hr at 40° C. Images were collected under a Leica TCS SP511 confocal microscope with LAS AF Lite software (Leica Microsystems).

The following RNAscope probes were used: Mm-Camk2a-C1 (#445231), Mm-Sc32a1-C1 (#319191), Mm-Sst-C1 (#404631), Mm-Pvalb-C2 (#421931), Mm-Vip-C2 (#502231), Mm-Rspo2-C2 (#402001), and Mm-Ppp1rb-C3 (#405901).

### REFERENCES

1. D. D. Price, Psychological and Neural Mechanisms of the Affective Dimension of Pain. Science (80). 288, 1769-1772 (2000).
2. M. N. Baliki, A. V. Apkarian, Nociception, Pain, Negative Moods, and Behavior Selection. Neuron. 87, 474-491 (2015).
3. C. Peirs, R. P. Seal, Neural circuits for pain: Recent advances and current views. Science (80). 354 (2016), doi:10.7554/eLife.22866.
4. L. Garcia-Larrea, H. Bastuji, Pain and consciousness. Prog. Neuro-Psychopharmacology Biol. Psychiatry (2017), doi:10.1016/j.pnpbp.2017.10.007.
5. P. H. Janak, K. M. Tye, From circuits to behaviour in the amygdala. Nature. 517, 284-292 (2015).
6. N. Hebben, S. Corkin, H. Eichenbaum, K. Shedlack, Diminished ability to interpret and report internal states after bilateral medial temporal resection: case H.M. Behav. Neurosci. 99, 1031-1039 (1985).
7. N. Neimann et al., Familial Urbach-Wiethe Disease with indifference to pain. Bull Soc Fr Dermatol Syphiligr. March, 292-4 (1964).
8. M. Ploner, H. Freund, A. Schnitzler, Pain affect without pain sensation in a patient with a postcentral lesion. Pain. 81, 211-4 (1999).
9. M. L. Uhelski, M. A. Davis, P. N. Fuchs, Pain affect in the absence of pain sensation: Evidence of asomaesthesia after somatosensory cortex lesions in the rat. Pain. 153, 885-892 (2012).
10. M. Costigan, J. Scholz, C. J. Woolf, Neuropathic Pain: A Maladaptive Response of the Nervous System to Damage. Annu. Rev. Neurosci. 32, 1-32 (2009).
11. V. Neugebauer, Amygdala Pain Mechanisms (2015), vol. 227.
12. P. Chang, M. Virginia, D. Procissi, A. Baria, A. V. Apkarian, Brain activity for tactile allodynia: a longitudinal awake rat functional magnetic resonance imaging study tracking emergence of neuropathic pain. 158, 488-497 (2017).
13. L. E. Simons et al., The responsive amygdala: Treatment-induced alterations in functional connectivity in pediatric complex regional pain syndrome. Pain (2014), doi:10.1016fj.pain.2014.05.023.
14. J. A. Hashmi et al., Shape shifting pain: Chronification of back pain shifts brain representation from nociceptive to emotional circuits. Brain. 136, 2751-2768 (2013).
15. F. Gore et al., Neural Representations of Unconditioned Stimuli in Basolateral Amygdala Mediate Innate and Learned Responses. Cell. 162, 134-145 (2015).
16. V. Neugebauer, W. Li, G. C. Bird, G. Bhave, R. W. Gereau, Synaptic plasticity in the amygdala in a model of arthritic pain: differential roles of metabotropic glutamate receptors 1 and 5. J. Neurosci. 23, 52-63 (2003).
17. S. Han et al., Elucidating an Affective Pain Circuit that Creates a Threat Memory Article Elucidating an Affective Pain Circuit that Creates a Threat Memory. Cell. 162, 363-374 (2015).
18. G. Ji et al., Cognitive impairment in pain through amygdala-driven prefrontal cortical deactivation. J. Neurosci. 30, 5451-5464 (2010).
19. B. F. Grewe et al., Neural ensemble dynamics underlying a long-term associative memory. Nature. 543, 670-675 (2017).
20. Y. Ziv et al., Long-term dynamics of CA1 hippocampal place codes SOM. Nat. Neurosci. 16,264-6 (2013).
21. J. G. Parker et al., Diametric neural ensemble dynamics in parkinsonian and dyskinetic states (2018), vol. 557.
22. W. Zhang et al., Functional circuits and anatomical distribution of response properties in the primate amygdala. J. Neurosci. 33, 722-33 (2013).
23. P. Namburi et al., A circuit mechanism for differentiating positive and negative associations. Nature. 520, 675-678 (2015).
24. J. Kim, M. Pignatelli, S. Xu, S. Itohara, S. Tonegawa, Antagonistic negative and positive neurons of the basolateral amygdala. Nat. Neurosci., 1-15 (2016).
25. C. Guenthner, K. Miyamichi, H. H. Yang, H. C. Heller, L. Luo, Permanent genetic access to transiently active neurons via TRAP: Targeted recombination in active populations. Neuron. 78, 773-784 (2013).
26. B. L. Roth, DREADDs for Neuroscientists. Neuron. 89, 683-694 (2016).
27. J. L. Gomez et al., Chemogenetics revealed: DREADD occupancy and activation via converted clozapine. Science (80). 357, 503-507 (2017).
28. G. G. Calhoon et al., Acute Food Deprivation Rapidly Modifies Valence-Coding Microcircuits in the Amygdala. bioRxiv (2018), doi:10.1101/285189.
29. C. A. Von Hehn, R. Baron, C. J. Woolf, Deconstructing the Neuropathic Pain Phenotype to Reveal Neural Mechanisms. Neuron. 73, 638-652 (2012).
30. B. W. Balleine, S. Killcross, Parallel incentive processing: an integrated view of amygdala function. Trends Neurosci. 29, 272-279 (2006).
31. C. D. Salzman, S. Fusi, Emotion, Cognition, and Mental State Representation in Amygdala and Prefrontal Cortex. Annu. Rev. Neurosci. 33, 173-202 (2010).
32. J. E. LeDoux, R. Brown, A higher-order theory of emotional consciousness. Proc. Natl. Acad. Sci. 114, E2016-E2025 (2017).
33. N. Grahek, Feeling Pain and Being in Pain (MIT Press, 2007).
34. R. Melzack, K. Casey, in The Skin Senses (Charles C. Thomas, 1968), pp. 432-439.
35. F. Ramirez, J. M. Moscarello, J. E. LeDoux, R. M. Sears, Active Avoidance Requires a Serial Basal Amygdala to Nucleus Accumbens Shell Circuit. J. Neurosci. 35, 3470-3477 (2015).
36. A. Burgos-robles et al., Amygdala inputs to prefrontal cortex guide behavior amid conflicting cues of reward and punishment. Nat. Neurosci. (2017), doi:10.1038/nn.4553.
37. A. Beyeler et al., Defined Neurons in the Basolateral Amygdala Article Organization of Valence-Encoding and Projection-Defined Neurons in the Basolateral Amygdala. Cell Rep. 22, 905-918 (2018).
38. K. Wiech, Deconstructing the sensation of pain: The influence of cognitive processes on pain perception. Science (80). 354, 584-587 (2016).
39. C. Torsney, A. B. MacDermott, Disinhibition Opens the Gate to Pathological Pain Signaling in Superficial Neurokinin 1 Receptor-Expressing Neurons in Rat Spinal Cord. J. Neurosci. 26, 1833-1843 (2006).
40. P. A. Pizzo, N. M. Clark, O. Carter Pokras, Relieving Pain in America: A Blueprint for Transforming Prevention, Care, Education, and Research (2011).
41. N. D. Volkow, F. S. Collins, The Role of Science in Addressing the Opioid Crisis. NEJM. 377, 391-394 (2017).
42. G. Corder, D. C. Castro, M. R. Bruchas, G. Scherrer, Endogenous and Exogenous Opioids in Pain. Annu. Rev. Neurosci. (2018), doi:10.1146/annurev-neuro-080317-061522.
43. P. Skolnick, N. D. Volkow, Re-energizing the Development of Pain Therapeutics in Light of the Opioid Epidemic. Neuron. 92, 294-297 (2016).
44. F. Jelasic, Relation of the lateral part of the amygdala to pain. Confin. Neurol. 21, 53-5 (1966).
45. B. T. Grosser, C. J. Woolf, G. A. Fitzgerald, Time for nonaddictive relief of pain. Science (80). 355, 1026-1027 (2017).
46. B. Ahanonu, calciumImagingAnalysis: a software package for analyzing one- and two-photon calcium imaging datasets. GitHub (2018), doi:10.5281/ZENODO.2222295
47. P. Jean-richard-dit-bressel, S. Killcross, G. P. Mcnally, Behavioral and neurobiological mechanisms of punishment: implications for psychiatric disorders. Neuropsychopharmacology (2018), doi:10.1038/s41386-018-0047-3.
48. R. Melzack, P. Wall, Pain Mechanisms: A New Theory. Science (80). 150, 971-979 (1965).
49. M. J. Millan, The induction of pain: an integrative review. Prog. Neurobiol. 57, 1-164 (1999).
50. G. Ekman, M. Frankenhauser, S. Levander, I. Mellis, The influence of intensity and duration of electrical stimulational on subjective variables. Scand. J. Psychol. 7, 58-64 (1966).
51. T. Tashiro, A. Higashiyama, The perceptual properties of electrocutaneous stimulation: Sensory quality, subjective intensity, and intensity-duration relation. Percept. Psychophys. 30, 579-586 (1982).
52. M. S. Fanselow, The postshock activity burst. Anim. Learn. Behav. 10, 448-454 (1982).
53. H. Beaudry, I. Daou, A. Ase, A. Riberiro-da-Silva, P. Seguela, Distinct behavioral responses evoked by selective optogenetic stimulation of the major TRPV1 1 and MrgD 1 subsets of C-fibers. Pain. 158 (2017).
54. D. Terburg et al., The Basolateral Amygdala Is Essential for Rapid Escape: A Human and Rodent Study. Cell. 175, 723-735.e16 (2018).
55. J. Fadok et al., A competitive inhibitory circuit for selection of active and passive fear responses. Nat. Publ. Gr. 542, 96-100 (2017).
56. C. Gauriau, J. Bernard, Pain pathways and parabrachial circuits in the rat. Exp. Physiol. 87, 251-8 (2002).
57. M. E. Carter, M. E. Soden, L. S. Zweifel, R. D. Palmiter, Genetic identification of a neural circuit that suppresses appetite. Nature. 503, 111-4 (2013).
58. R. D. Palmiter, The Parabrachial Nucleus: CGRP Neurons Function as a General Alarm. Trends Neurosci. 41, 280-293 (2018).
59. C. A. Campos, A. J. Bowen, C. W. Roman, R. D. Palmiter, Encoding of danger by parabrachial CGRP neurons. Nature. 555, 617-620 (2018).
60. P. Tovote et al., Mibrain circuits for defensive behaviour. Nature. 534, 206-212 (2016).
61. T. Ozawa et al., A feedback neural circuit for calibrating aversive memory strength. Nat. Neurosci. 20, 90-97 (2016).
62. R. J. Fox, C. A. Sorenson, Bilateral lesions of the amygdala attenuate analgesia induced by diverse environmental challenges. Brain Res. 648, 215-221 (1994).
63. W. Zhu, Z. Z. Pan, mu-Opioid-Mediated Inhibition of Glutamate Synaptic Transmission in Rat Central Amygdala Neurons. Neuroscience. 133, 97-103 (2005).
64. M. A. Oliveira, W. A. Prado, Role of PAG in the antinociception evoked from the medial or central amygdala in rats. Brain Res. Bull. 54, 55-63 (2001).
65. J. Annese et al., Postmortem examination of patient H.M.'s brain based on histological sectioning and digital 3D reconstruction. Nat Commun. 5, 3122 (2014).
66. P. Veinante, I. Yalcin, M. Barrot, The amygdala between sensation and affect: a role in pain. J. Mol. Psychiatry. 1, 9 (2013).
67. P. Kyriazi, D. B. Headley, D. Pare, Multi-dimensional Coding by Basolateral Amygdala Neurons. Neuron. 99, 1315-1328.e5 (2018).
68. T.-W. Chen et al., Ultrasensitive fluorescent proteins for imaging neuronal activity. Nature. 499, 295-300 (2013).
69. B. Slotnick, A Simple 2-Transistor Touch or Lick Detector Circuit. J. Exp. Anal. Behav. 91, 253-255 (2009).
70. B. Solway, S. C. Bose, G. Corder, R. R. Donahue, B. K. Taylor, Tonic inhibition of chronic pain by neuropeptide Y. Proc. Natl. Acad. Sci. U.S.A 108, 7224-7229 (2011).
71. K. Kobayakawa et al., Innate versus learned odour processing in the mouse olfactory bulb. Nature. 450, 503-508 (2007).
72. C. M. Root, C. A. Denny, R. Hen, R. Axel, The participation of cortical amygdala in innate, odour-driven behaviour. Nature. 515, 269-273 (2014).
73. L. R. Saraiva et al., Combinatorial effects of odorants on mouse behavior. Proc. Natl. Acad. Sci. 113, E3300-E3306 (2016).
74. Y. A. Blednov, B. F. Cravatt, S. L. Boehm, D. Walker, R. A. Harris, Role of endocannabinoids in alcohol consumption and intoxication: Studies of mice lacking fatty acid amide hydrolase. Neuropsychopharmacology. 32, 1570-1582 (2007).
75. P. Thévenaz, U. E. Ruttimann, M. Unser, A pyramid approach to subpixel registration based on intensity. IEEE Trans. Image Process. 7, 27-41 (1998).
76. E. A. Mukamel, A. Nimmerjahn, M. J. Schnitzer, Automated Analysis of Cellular Signals from Large-Scale Calcium Imaging Data. Neuron. 63, 747-760 (2009).
77. Y. Benjamini, Y. Hochberg, Controlling the false discovery rate: a practical and powerful approach to multiple testing. J. R. Stat. Soc. B. 57 (1995), pp. 289-300.
78. M. J. Mathie, N. H. Lovell, A. C. F. Coster, B. G. Celler, Determining activity using a triaxial accelerometer. Proc. Second Jt. 24th Annu. Conf. Annu. Fall Meet. Biomed. Eng. Soc., 2481-2482 (2002).
79. Y. Li et al., Neuronal Representation of Social Information in the Medial Amygdala of Awake Behaving Mice. Cell. 171, 1176-1179.e17 (2017).
80. S. M. Iyer et al., Virally mediated optogenetic excitation and inhibition of pain in freely moving nontransgenic mice. Nat. Biotechnol. 32, 274-278 (2014).
81. G. Corder et al., Loss of μ-opioid receptor signaling in nociceptors, and not spinal microglia, abrogates morphine tolerance without disrupting analgesic efficacy. Nat. Med. 23, 164-173 (2017).
82. A. Manglik et al., Structure-based discovery of opioid analgesics with reduced side effects. Nature, 1-6 (2016).
83. C. J. Woolf, Long term alterations in the excitability of the flexion reflex produced by peripheral tissue injury in the chronic decerebrate rat. Pain. 18, 325-343 (1984).
84. R. Blanchard, D. Blanchard, Passive and active reactions to fear-eliciting stimuli. J Comp Physiol Psychol. 68, 129-135 (1969).
85. R. Bolles, Species-specific defense reactions and avoidance learning. Psychol Rev. 77, 32-48 (1970).
86. Bolles, M. Fanselow, A perceptual-defensive-recuperative model of fear and pain. Behav Brain Sci. 3, 291-323 (1980).
87. C. Darwin, The expression of the emotions in man and animals (Albemarle, London, 1872).
88. W. Estes, Discriminative conditioning; effects of a Pavlovian conditioned stimulus upon a subsequently established operant response. J Exp Psychol. 38, 173-177 (1948).
89. W. Estes, B. Skinner, Some quantitative properties of anxiety. J Exp Psychol. 29, 390-400 (1941).
90. J. S. Mogil, Animal models of pain: progress and challenges. Nat. Rev. Neurosci. 10, 283-294 (2009).
91. R. Rescola, Pavlovian conditioned fear in Sidman avoidance learning. J Comp Physiol Psychol. 65, 55-60 (1968).
92. B. Skinner, The behavior of organisms: an experimental analysis. Appleton-CenturyCrofts, New York (1938).
93. S. R. Chaplan, F. W. Bach, J. W. Pogrel, J. M. Chung, T. L. Yaksh, Quantitative assessment of tactile allodynia in the rat paw. J. Neurosci. Methods. 53, 55-63 (1994).
94. G. Corder et al., Constitutive μ-opioid receptor activity leads to long-term endogenous analgesia and dependence. Science (80). 341, 1394-1399 (2013).

TABLE 1
Summary of the percent of stimuli responsive basolateral amygdala neurons in neuropathic and sham groups

| | | | | | | | % of nociceptive | |
|---|---|---|---|---|---|---|---|---|
| | | | | | | | ensemble as function of | |
| | | | % of total cells | | % of nociceptive ensemble | | % total cells | |
| Stimulus | Mice | Group | Uninjured | Neuropathic | Uninjured | Neuropathic | Uninjured | Neuropathic |
| Noxious heat (−55° C. water) | FIGS. 7A, 7C² | Neuropathic | 13 ± 2 | 18 ± 2 | 56 ± 8 | 53 ± 4 | 13 ± 2 | 18 ± 2 |
| Noxious cold (−5° C. water | FIGS. 7A, 7C² | Neuropathic | 15 ± 2 | 22 ± 3 | 60 ± 5 | 66 ± 3 | 15 ± 2 | 22 ± 3 |
| or Acetone) | | | | | | | | |
| Noxious pin (Pin prick) | FIGS. 7A, 7C² | Neuropathic | 16 ± 3 | 19 ± 2 | 61 ± 6 | 59 ± 3 | 16 ± 3 | 19 ± 2 |
| Mild touch (2.0 g filament) | FIGS. 7A, 7C² | Neuropathic | 12 ± 2 | 17 ± 2 | 36 ± 5 | 42 ± 3 | 9 ± 2 | 15 ± 2 |
| Light touch (0.07 g filament) | FIGS. 7A, 7C² | Neuropathic | 7 ± 2 | 14 ± 2 | 21 ± 4 | 33 ± 3 | 6 ± 1 | 11 ± 2 |
| Approach (“Miss hit” or | FIGS. 7A, 7C² | Neuropathic | 9 ± 3 | 12 ± 2 | 20 ± 4 | 24 ± 3 | 4 ± 1 | 8 ± 2 |
| no contact) | | | | | | | | |
| Noise | FIGS. 7A, 7C² | Neuropathic | 37 ± 5 | 39 ± 4 | 68 ± 5 | 59 ± 4 | 17 ± 3 | 21 ± 3 |
| 10% sucrose | FIGS. 7A, 7C² | Neuropathic | 19 ± 4 | 17 ± 3 | 25 ± 6 | 20 ± 3 | 7 ± 2 | 7 ± 2 |
| Background | FIGS. 7A, 7C² | Neuropathic | 0.1 ± 0.1 | 0.5 ± 0.2 | 0.3 ± 0.2 | 0.3 ± 0.2 | 0.1 ± 0.1 | 0.1 ± 0.1 |
| Nociceptive ensemble¹ | FIGS. 7A, 7C² | Neuropathic | 25 ± 3 | 32 ± 2 | 100 ± 0 | 100 ± 0 | 25 ± 3 | 32 ± 2 |
| Noxious heat (−55° C. water) | FIG. 7C ³ | Sham | 14 ± 4 | 9 ± 1 | 44 ± 9 | 47 ± 5 | 14 ± 4 | 9 ± 1 |
| Noxious cold (−5° C. water | FIG. 7C ³ | Sham | 10 ± 1 | 10 ± 1 | 54 ± 8 | 51 ± 5 | 10 ± 1 | 10 ± 1 |
| or Acetone) | | | | | | | | |
| Pin prick | FIG. 7C ³ | Sham | 15 ± 4 | 9 ± 1 | 56 ± 6 | 45 ± 5 | 15 ± 4 | 9 ± 1 |
| Mild touch (2.0 g filament) | FIG. 7C ³ | Sham | 7 ± 1 | 4 ± 1 | 24 ± 5 | 16 ± 3 | 6 ± 1 | 3 ± 1 |
| Light touch (0.07 g filament) | FIG. 7C ³ | Sham | 7 ± 3 | 2 ± 0 | 15 ± 5 | 6 ± 1 | 5 ± 2 | 1 ± 0 |
| Approach (“Miss hit” or | FIG. 7C ³ | Sham | 4 ± 1 | 5 ± 1 | 9 ± 3 | 10 ± 3 | 3 ± 1 | 2 ± 1 |
| no contact) | | | | | | | | |
| Noise | FIG. 7C ³ | Sham | 28 ± 5 | 25 ± 3 | 60 ± 6 | 44 ± 5 | 14 ± 3 | 8 ± 1 |
| 10% sucrose | FIG. 7C ³ | Sham | 16 ± 5 | 11 ± 2 | 26 ± 7 | 19 ± 4 | 8 ± 3 | 3 ± 1 |
| Background | FIG. 7C ³ | Sham | 0.4 ± 0.3 | 0.2 ± 0.1 | 0.1 ± 0.1 | 0.2 ± 0.2 | 0 ± 0 | 0 ± 0 |
| Nociceptive ensemble¹ | FIG. 7C ³ | Sham | 24 ± 4 | 18 ± 2 | 100 ± 0 | 100 ± 0 | 24 ± 4 | 18 ± 2 |
| Noxious cold (Acetone) | FIG. 7B ⁵ | Neuropathic | 20 ± 4 | 14 ± 2 | 63 ± 4 | 57 ± 4 | 20 ± 4 | 14 ± 2 |
| Noxious pin (Pin prick) | FIG. 7B ⁵ | Neuropathic | 21 ± 4 | 19 ± 4 | 66 ± 4 | 67 ± 4 | 21 ± 4 | 19 ± 4 |
| Mild touch (2.0 g filament) | FIG. 7B ⁵ | Neuropathic | 17 ± 3 | 18 ± 3 | 29 ± 5 | 37 ± 6 | 12 ± 3 | 13 ± 3 |
| Light touch (0.07 g filament) | FIG. 7B ⁵ | Neuropathic | 10 ± 4 | 16 ± 3 | 14 ± 5 | 30 ± 6 | 7 ± 3 | 10 ± 2 |
| Nociceptive ensemble⁴ | FIG. 7B ⁵ | Neuropathic | 28 ± 4 | 25 ± 4 | 100 ± 0 | 100 ± 0 | 28 ± 4 | 25 ± 4 |

¹Consist of cells responsive to 55° C. water, 5° C. water, Acetone, or Pin prick.
²N = 5 mice, analysis from 3 or 4 (uninjured) and 5 or 7 (neuropathic) sessions per mice. All values mean ± s.e.m.
³ N = 4 mice, analysis from 3 (uninjured) and 7 (sham surgery) sessions per mice. All values mean ± s.e.m.
⁴Consist of cells responsive to Acetone and Pin prick.
⁵ N = 8 mice, analysis from 22 (uninjured) and 26 (injured) total sessions pooled across all mice. All values mean ± s.e.m.

Table 1. Associated data for FIGS. 1 and 7. Last two columns (“% of nociceptive ensemble as a function of % total neurons”) are a measure of columns 5 and 6 (“% of nociceptive ensemble”) neurons within the total population. FIGS. 7A, 7C neuropathic mice have 1,779 neurons [5 mice, 3-4 sessions each] and 3,783 neurons [5 mice, 5-7 sessions each] from normal and neuropathic sessions, respectively. FIG. 7C sham mice have 1,618 neurons [4 mice, 3 sessions each] and 3,752 neurons [4 mice, 7 sessions each] from normal and uninjured sessions, respectively. FIG. 7B neuropathic mice have n=2,839 [8 mice, 22 total sessions] and 3,625 [8 mice, 26 total sessions] neurons from normal and neuropathic sessions, respectively.

1. A method of screening for an agent that modulates neural activity in a basolateral amygdala (BLA) nociceptive ensemble in a brain of a subject, the method comprising:
a) contacting the BLA nociceptive ensemble with a candidate agent; and
b) measuring neural activity in the BLA nociceptive ensemble in response to the candidate agent.

2. The method of claim 1, further comprising monitoring pain perception in the subject to determine if the candidate agent modulates pain perception.

3. The method of claim 2, wherein pain perception is monitored in response to a test stimulus.

4. The method of claim 3, wherein the test stimulus is a noxious stimulus or an innocuous stimulus.

5. The method of claim 4, wherein the noxious stimulus is a noxious thermal stimulus or a noxious mechanical stimulus.

6. The method of claim 5, wherein the noxious thermal stimulus is noxious heat or noxious cold.

7. The method of claim 5, wherein the noxious mechanical stimulus is a noxious pin prick or filament.

8. The method of claim 4, wherein the innocuous stimulus is light touch.

9. The method of claim 2, wherein reduced pain perception in response to the noxious stimulus in the presence of the candidate agent compared to in absence of the candidate agent indicates that the candidate agent has analgesic activity.

10. The method of claim 1, further comprising monitoring the subject for reduced pain affective-motivational behavior in the presence of the candidate agent compared to in the absence of the candidate agent.

11. The method of claim 1, wherein the candidate agent is a small molecule, a peptide, a protein, a ligand, an aptamer, an antibody, an antibody mimetic, or an inhibitory nucleic acid that modulates neural activity of at least a subset of neurons in the BLA nociceptive ensemble.

12. The method of claim 11, wherein the antibody is selected from the group consisting of a polyclonal antibody, a monoclonal antibody, a chimeric antibody, a humanized antibody, a F(ab) fragment, a F(ab′)2 fragment, a Fᵥ fragment, and a nanobody.

13. The method of claim 11, wherein the inhibitory nucleic acid is selected from the group consisting of a small interfering RNA (siRNA), a microRNA (miRNA), a Piwi-interacting RNA (piRNA), a small nuclear RNA (snRNA), an antisense oligonucleotide, and a peptide nucleic acid.

14. The method of claim 2, wherein pain perception is monitored in the subject using a mechanical withdrawal test, an electronic Von Frey test, a manual Von Frey test, a Randall-Selitto test, a Hargreaves test, a hot plate test, a cold plate test, a thermal probe test, an acetone evaporation test, cold plantar test, a temperature preference test, a grimace scale test, or weight bearing and gait analysis.

15. The method of claim 1, wherein the agent disrupts neural activity of a subset of neurons in the BLA nociceptive ensemble.

16. The method of claim 15, wherein the subset comprises or consists of a nociceptive-specific subpopulation of neurons.

17. The method of claim 1, wherein the agent disrupts neural activity of all of the neurons of the BLA nociceptive ensemble.

18. A method of mapping nociceptive and aversive responses to neurons in a basolateral amygdala (BLA) nociceptive ensemble in the brain of a subject, the method comprising:
a) imaging neural activity within the BLA nociceptive ensemble associated with nociceptive and aversive responses to a test stimulus; and
b) mapping responsive neurons exhibiting the neural activity.

19. The method of claim 18, wherein the neural activity is Ca²⁺ transient activity of one or more neurons in the BLA nociceptive ensemble.

20. A method of treating a subject for pain, the method comprising locally administering a therapeutically effective amount of an agent that disrupts neural activity in a basolateral amygdala (BLA) nociceptive ensemble in the brain of the subject.

21. The method of claim 20, wherein the agent disrupts neural activity of a subset of neurons in the BLA nociceptive ensemble.

22. The method of claim 21, wherein the subset comprises or consists of a nociceptive-specific subpopulation of neurons.

23. The method of claim 20, wherein the agent disrupts neural activity of all of the neurons in the BLA nociceptive ensemble.

24. The method of claim 20, wherein the agent is administered in an amount sufficient to attenuate neuropathic pain or pathological pain.

25. The method of claim 1, wherein the agent is administered in an amount sufficient to relieve allodynia or hyperalgesia.

26. The method of claim 25, wherein the allodynia or the hyperalgesia is thermal, mechanical, or opioid-induced allodynia or hyperalgesia.

27. The method of claim 20, wherein the agent is administered in an amount sufficient to reduce aversive pain avoidance behavior.

28. The method of claim 20, wherein the nociceptive ensemble comprises c-Fos⁺ mid-anterior BLA Camk2a⁺ principal neurons that are activated by nociceptive stimuli.

29. The method of claim 20, wherein the nociceptive ensemble comprises a nociceptive-specific subpopulation of neurons.

30. The method of claim 20, wherein the pain is acute pain or chronic pain.

31. The method of claim 20, wherein the agent is administered by stereotactic injection into the BLA nociceptive ensemble in the brain of the subject.
